SEQUENCE LISTING (57 sequences)

SEQ ID NO: 1 - protein, 124 aa, Artificial Sequence
  Synthetic Polypeptide
DLGKKLLEAARAGQDDEVRELLKAGADVNAKDYFSHTPLHLAARNGHLKIVEVLLKAGADVNAKDFAGKTPLHLAAADGHLEIVEVLLKAGADVNAQDIFGKTPADIAADAGHEDIAEVLQKAA

SEQ ID NO: 2 - protein, 157 aa, Artificial Sequence
  Synthetic Polypeptide
DLGKKLLEAARAGQDDEVRELLKAGADVNAKDVLGWTPLHLAAFEGHLEIVEVLLKAGADVNAKDKKGWTPLQLAARTGHLEIVEVLLKAGADVNAKDHIGATPLHLAAWQGHPEIVEVLLKAGADVNAQDKSGKTPADLAADAGHEDIAEVLQKAA

SEQ ID NO: 3 - protein, 124 aa, Artificial Sequence
  Synthetic Polypeptide
DLGQKLLKAAQEGQDDEVRELLKAGADVNAKDLRGITPLHVAAWQGHLEIVEVLLKAGADVNAKDSKGETPLHLAAFRGHLEIVEVLLKAGADVNAQDQQGETPADLAALAGHVDIAEVLQKAA

SEQ ID NO: 4 - protein, 24 aa, Artificial Sequence
  Synthetic Polypeptide
GSPTPTPTTPTPTPTTPTPTPTGS

SEQ ID NO: 5 - protein, 126 aa, Artificial Sequence
  Synthetic Polypeptide
GSDLGKKLLEAARAGQDDEVRELLKAGADVNAKDYFSHTPLHLAARNGHLKIVEVLLKAGADVNAKDFAGKTPLHLAAADGHLEIVEVLLKAGADVNAQDIFGKTPADIAADAGHEDIAEVLQKAA

SEQ ID NO: 6 - protein, 751 aa, Artificial Sequence
  Synthetic Polypeptide
GSDLGKKLLEAARAGQDDEVRELLKAGADVNAKDYFSHTPLHLAARNGHLKIVEVLLKAGADVNAKDFAGKTPLHLAAADGHLEIVEVLLKAGADVNAQDIFGKTPADIAADAGHEDIAEVLQKAAGSPTPTPTTPTPTPTTPTPTPTGSDLGKKLLEAARAGQDDEVRELLKAGADVNAKDVLGWTPLHLAAFEGHLEIVEVLLKAGADVNAKDKKGWTPLQLAARTGHLEIVEVLLKAGADVNAKDHIGATPLHLAAWQGHPEIVEVLLKAGADVNAQDKSGKTPADLAADAGHEDIAEVLQKAAGSPTPTPTTPTPTPTTPTPTPTGSDLGQKLLKAAQEGQDDEVRELLKAGADVNAKDLRGITPLHVAAWQGHLEIVEVLLKAGADVNAKDSKGETPLHLAAFRGHLEIVEVLLKAGADVNAQDQQGETPADLAALAGHVDIAEVLQKAAGSPTPTPTTPTPTPTTPTPTPTGSDLGQKLLKAAQEGQDDEVRELLKAGADVNAKDLRGITPLHVAAWQGHLEIVEVLLKAGADVNAKDSKGETPLHLAAFRGHLEIVEVLLKAGADVNAQDQQGETPADLAALAGHVDIAEVLQKAAGSPTPTPTTPTPTPTTPTPTPTGSDLGKKLLEAARAGQDDEVRELLKAGADVNAKDYFSHTPLHLAARNGHLKIVEVLLKAGADVNAKDFAGKTPLHLAAADGHLEIVEVLLKAGADVNAQDIFGKTPADIAADAGHEDIAEVLQKAA

SEQ ID NO: 7 - protein, 32 aa, Artificial Sequence
  Synthetic Polypeptide
GSDLGKKLLEAARAGQDDEVRILMANGADVNA

SEQ ID NO: 8 - protein, 28 aa, Artificial Sequence
  Synthetic Polypeptide
QDKFGKTAFDISIDNGNEDLAEILQKLN

SEQ ID NO: 9 - protein, 32 aa, Artificial Sequence
  Synthetic Polypeptide
GSDLGKKLLEAARAGQDDEVRILLKAGADVNA

SEQ ID NO: 10 - protein, 32 aa, Artificial Sequence
  Synthetic Polypeptide
GSDLGKKLLEAARAGQDDEVRELLKAGADVNA

SEQ ID NO: 11 - protein, 33 aa, Artificial Sequence
  Synthetic Polypeptide
  MISC_FEATURE (2)..(3): xaa is any amino acid (preferably not cysteine, glycine, or proline)
  MISC_FEATURE (5)..(5): xaa is any amino acid (preferably not cysteine, glycine, or proline)
  MISC_FEATURE (12)..(14): xaa is any amino acid (preferably not cysteine, glycine, or proline)
  MISC_FEATURE (16)..(18): xaa is any amino acid (preferably not cysteine, glycine, or proline)
  MISC_FEATURE (21)..(21): xaa is any amino acid (preferably not cysteine, glycine, or proline)
  MISC_FEATURE (25)..(26): xaa is any amino acid (preferably not cysteine, glycine, or proline)
  MISC_FEATURE (33)..(33): xaa is any amino acid (preferably not cysteine, glycine, or proline)
DXXGXTPLHLAXXXGXXXLVXVLLXXGADVNAX

SEQ ID NO: 12 - protein, 33 aa, Artificial Sequence
  Synthetic Polypeptide
  MISC_FEATURE (2)..(3): xaa is any amino acid (preferably not cysteine, glycine, or proline)
  MISC_FEATURE (5)..(5): xaa is any amino acid (preferably not cysteine, glycine, or proline)
  MISC_FEATURE (13)..(14): xaa is any amino acid (preferably not cysteine, glycine, or proline)
  MISC_FEATURE (26)..(26): xaa is asparagine, histidine, or tyrosine
  MISC_FEATURE (33)..(33): xaa is any amino acid (preferably not cysteine, glycine, or proline)
DXXGXTPLHLAAXXGHLEIVEVLLKXGADVNAX

SEQ ID NO: 13 - protein, 255 aa, Artificial Sequence
  Synthetic Polypeptide
MGNSCYNIVATLLLVLNFERTRSLQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSPQIISFFLALTSTALLFLLFFLTLRFSVVKRGRKKLLYIFKQPFMRPVQTTQEEDGCSCRFPEEEEGGCEL

SEQ ID NO: 14 - protein, 760 aa, Artificial Sequence
  Synthetic Polypeptide
MKTWVKIVFGVATSAVLALLVMCIVLRPSRVHNSEENTMRALTLKDILNGTFSYKTFFPNWISGQEYLHQSADNNIVLYNIETGQSYTILSNRTMKSVNASNYGLSPDRQFVYLESDYSKLWRYSYTATYYIYDLSNGEFVRGNELPRPIQYLCWSPVGSKLAYVYQNNIYLKQRPGDPPFQITFNGRENKIFNGIPDWVYEEEMLATKYALWWSPNGKFLAYAEFNDTDIPVIAYSYYGDEQYPRTINIPYPKAGAKNPVVRIFIIDTTYPAYVGPQEVPVPAMIASSDYYFSWLTWVTDERVCLQWLKRVQNVSVLSICDFREDWQTWDCPKTQEHIEESRTGWAGGFFVSTPVFSYDAISYYKIFSDKDGYKHIHYIKDTVENAIQITSGKWEAINIFRVTQDSLFYSSNEFEEYPGRRNIYRISIGSYPPSKKCVTCHLRKERCQYYTASFSDYAKYYALVCYGPGIPISTLHDGRTDQEIKILEENKELENALKNIQLPKEEIKKLEVDEITLWYKMILPPQFDRSKKYPLLIQVYGGPCSQSVRSVFAVNWISYLASKEGMVIALVDGRGTAFQGDKLLYAVYRKLGVYEVEDQITAVRKFIEMGFIDEKRIAIWGWSYGGYVSSLALASGTGLFKCGIAVAPVSSWEYYASVYTERFMGLPTKDDNLEHYKNSTVMARAEYFRNVDYLLIHGTADDNVHFQNSAQIAKALVNAQVDFQAMWYSDQNHGLSGLSTNHLYTHMTHFLKQCFSLSD

SEQ ID NO: 15 - protein, 585 aa, Artificial Sequence [sequence omitted]

SEQ ID NO: 16 - protein, 4 aa, Artificial Sequence
  Synthetic Polypeptide
  REPEAT (1)..(4): Can repeat 1-6 times
GGGS

SEQ ID NO: 17 - DNA, 2256 bp, Artificial Sequence
  Synthetic Polynucleotide
atgggatccgatctgggtaaaaagctgctggaggcagcgcgtgccggtcaagacgacgaggttcgcgaattgcttaaagcgggtgcagacgtcaacgccaaagattatttctctcataccccgttgcatttagccgcgcgtaatggccatctgaagatcgtcgaggtcctcttgaaggcaggcgcggatgtcaatgcgaaggattttgcgggcaaaacgccgctgcacttagcggcggcggacggtcatttagaaatcgttgaagtcctgttaaaagcgggcgccgatgtgaatgcgcaggatattttcggtaaaacgccggcggacattgcggcagatgcgggtcatgaagatatcgcagaagtcctgcaaaaagcagcgggttccccgacgccaacgccgaccaccccgaccccaactccgacgaccccgactccgaccccgaccggttctgatttaggcaagaagctgttggaggcggcccgtgctggccaggacgacgaagtccgtgagctgctgaaagcgggtgcggacgtgaatgccaaagacgtgttgggctggacccctctgcatctggctgccttcgagggccacctggagattgtggaagtccttctcaaggctggcgctgatgtgaacgcaaaggacaaaaagggttggactccgctgcagctggcggcgcgtaccggtcacctcgaaattgtagaggtcctgttgaaggcgggcgcagatgtaaatgctaaagaccatattggcgcaactccgctccacctggcggcatggcagggccacccagagatcgtggaggtcctcctgaaggcaggcgccgacgtgaacgcccaagacaaatccggtaagactccggcggacctggctgccgacgcaggccatgaggacatcgcggaagtcttgcagaaggcggctggcagccctaccccaaccccaaccacgccaactccaaccccgactacgccgaccccgacgccgaccggcagcgatctgggtcaaaaactgttgaaagctgcgcaagagggtcaagatgatgaggtgcgtgaactgctgaaagccggtgcggatgttaacgccaaggatttgcgcggtatcacgccactgcatgtggcggcgtggcaaggtcaccttgaaattgtggaggttcttttgaaggccggtgcagatgtcaatgcaaaagattccaaaggcgagactccactgcacctggcagcttttcgtggtcacttggaaattgtcgaggtattgctcaaagcgggtgccgatgttaatgcacaggaccaacagggtgagaccccggcggatttggcggccctggcgggccacgttgatatcgctgaagtgttgcaaaaagccgcgggctcaccgactcctactccgactacgccgaccccgacgcctacgaccccaacgcctacccctacgggcagcgacttgggtcagaagttgttgaaagcggcacaggagggtcaggacgatgaggtccgtgaattgctgaaggccggcgcagacgtgaatgcaaaggatctgcgtggcattaccccgctgcacgtggcggcctggcagggtcacctggaaattgttgaggttttactgaaagctggcgcggacgttaacgctaaagacagcaaaggtgaaaccccgttacatctggcggcattccgtggccacttggagatcgttgaggtgctgctgaaggctggtgccgacgtcaatgctcaagatcaacaaggtgaaacgccagcagacttggcagcgctggctggtcatgtcgatatcgcggaggtactgcagaaagcggcgggtagcccgacccctaccccgacgacgccaacccctacgccgacgactccgacgccgacgccgactggttcggacctgggtaagaaactgctggaagcggctcgcgcaggccaggatgacgaagtgcgtgagctgttaaaggccggtgctgatgtcaacgcgaaggactatttctcccataccccgctgcatttggcggctcgtaacggtcacctgaaaatcgtggaagtgttgctgaaagcaggcgcagacgttaatgccaaggacttcgccggtaaaactccgttacacttggcggcggctgatggccatcttgagattgtcgaagttctgctcaaggcgggtgctgacgtaaatgcgcaagacattttcggcaagacgccggcagacatcgcggcagacgccggccacgaggacattgctgaggtgctccaaaaggcagca

SEQ ID NO: 18 - protein, 157 aa, Artificial Sequence
  Synthetic Polypeptide
DLGKKLLEAARAGQDDEVRILLAAGADVNAKDVLGWTPLHLAAFEGHLEIVEVLLKAGADVNAKDKKGWTPLQLAARTGHLEIVEVLLKAGADVNAKDHIGATPLHLAAWQGHPEIVEVLLKAGADVNAQDKSGKTPADLAADAGHEDIAEVLQKAA

SEQ ID NO: 19 - protein, 157 aa, Artificial Sequence
  Synthetic Polypeptide
DLGKKLLEAARAGQDDEVRILLKAGADVNAKDVLGWTPLHLAAFEGHLEIVEVLLKAGADVNAKDKKGWTPLQLAARTGHLEIVEVLLKAGADVNAKDHIGATPLHLAAWQGHPEIVEVLLKAGADVNAQDKSGKTPADLAADAGHEDIAEVLQKAA

SEQ ID NO: 20 - protein, 157 aa, Artificial Sequence
  Synthetic Polypeptide
DLGKKLLEAARAGQDDEVRELLKAGADVNAKDVLGWTPLHLAAFEGHLEIVEVLLKAGADVNAKDKKGWTPLQLAARTGHLEIVEVLLKAGADVNAKDHIGATPLHLAAWQGHLEIVEVLLKAGADVNAQDKSGKTPADLAADAGHEDIAEVLQKAA

SEQ ID NO: 21 - protein, 157 aa, Artificial Sequence
  Synthetic Polypeptide
DLGKKLLEAARAGQDDEVRILLKAGADVNAKDVLGWTPLHLAAFEGHLEIVEVLLKAGADVNAKDKKGWTPLQLAARTGHLEIVEVLLKAGADVNAKDHIGATPLHLAAWQGHLEIVEVLLKAGADVNAQDKSGKTPADLAADAGHEDIAEVLQKAA

SEQ ID NO: 22 - protein, 157 aa, Artificial Sequence
  Synthetic Polypeptide
DLGKKLLEAARAGQDDEVRILLAAGADVNAKDVLGWTPLHLAAFEGHLEIVEVLLKAGADVNAKDKKGWTPLQLAARTGHLEIVEVLLKAGADVNAKDHIGATPLHLAAWQGHLEIVEVLLKAGADVNAQDKSGKTPADLAADAGHEDIAEVLQKAA

SEQ ID NO: 23 - protein, 157 aa, Artificial Sequence
  Synthetic Polypeptide
DLGKKLLEAARAGQDDEVRELLKAGADVNAKDVLGWTPLHLAAFEGHLEIVEVLLKAGADVNAKDKKGWTPLQLAARTGHLEIVEVLLKAGADVNAKDHIGATPLHLAAWQGHPEIVEVLLKAGADVNAQDKSGKTPADLAADAGHEDIAEVLQKLN

SEQ ID NO: 24 - protein, 124 aa, Artificial Sequence
  Synthetic Polypeptide
DLGQKLLKAAQEGQDDEVRELLKAGADVNAKDLRGITPLHVAAWQGHLEIVEVLLKAGADVNAKDSKGETPLHLAAFRGHLEIVEVLLKAGADVNAQDQQGETPADLAALAGHEDIAEVLQKAA

SEQ ID NO: 25 - protein, 124 aa, Artificial Sequence
  Synthetic Polypeptide
DLGQKLLKAAQEGQDDEVRILLKAGADVNAKDLRGITPLHVAAWQGHLEIVEVLLKAGADVNAKDSKGETPLHLAAFRGHLEIVEVLLKAGADVNAQDQQGETPADLAALAGHEDIAEVLQKAA

SEQ ID NO: 26 - protein, 124 aa, Artificial Sequence
  Synthetic Polypeptide
DLGQKLLKAAQEGQDDEVRILLAAGADVNAKDLRGITPLHVAAWQGHLEIVEVLLKAGADVNAKDSKGETPLHLAAFRGHLEIVEVLLKAGADVNAQDQQGETPADLAALAGHEDIAEVLQKAA

SEQ ID NO: 27 - protein, 124 aa, Artificial Sequence
  Synthetic Polypeptide
DLGQKLLKAAQEGQDDEVRILLKAGADVNAKDLRGITPLHVAAWQGHLEIVEVLLKAGADVNAKDSKGETPLHLAAFRGHLEIVEVLLKAGADVNAQDQQGETPADLAALAGHVDIAEVLQKAA

SEQ ID NO: 28 - protein, 124 aa, Artificial Sequence
  Synthetic Polypeptide
DLGQKLLKAAQEGQDDEVRILLAAGADVNAKDLRGITPLHVAAWQGHLEIVEVLLKAGADVNAKDSKGETPLHLAAFRGHLEIVEVLLKAGADVNAQDQQGETPADLAALAGHVDIAEVLQKAA

SEQ ID NO: 29 - protein, 124 aa, Artificial Sequence
  Synthetic Polypeptide
DLGQKLLKAAQEGQDDEVRELLKAGADVNAKDLRGITPLHVAAWQGHLEIVEVLLKAGADVNAKDSKGETPLHLAAFRGHLEIVEVLLKAGADVNAQDQQGETPADLAALAGHVDIAEVLQKLN

SEQ ID NO: 30 - protein, 124 aa, Artificial Sequence
  Synthetic Polypeptide
DLGKKLLEAARAGQDDEVRELLKAGADVNAKDYFSHTPLHLAARNGHLKIVEVLLKAGADVNAKDFAGKTPLHLAANEGHLEIVEVLLKAGADVNAQDIFGKTPADIAADAGHEDIAEVLQKAA

SEQ ID NO: 31 - protein, 124 aa, Artificial Sequence
  Synthetic Polypeptide
DLGKKLLEAARAGQDDEVRELLKAGADVNAKDYFSHTPLHLAARNGHLKIVEVLLKAGADVNAKDFAGKTPLHLAADAGHLEIVEVLLKAGADVNAQDIFGKTPADIAADAGHEDIAEVLQKAA

SEQ ID NO: 32 - protein, 163 aa, Artificial Sequence
  Synthetic Polypeptide
LQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSPQ

SEQ ID NO: 33 - protein, 735 aa, Artificial Sequence
  Synthetic Polypeptide
LRPSRVHNSEENTMRALTLKDILNGTFSYKTFFPNWISGQEYLHQSADNNIVLYNIETGQSYTILSNRTMKSVNASNYGLSPDRQFVYLESDYSKLWRYSYTATYYIYDLSNGEFVRGNELPRPIQYLCWSPVGSKLAYVYQNNIYLKQRPGDPPFQITFNGRENKIFNGIPDWVYEEEMLATKYALWWSPNGKFLAYAEFNDTDIPVIAYSYYGDEQYPRTINIPYPKAGAKNPVVRIFIIDTTYPAYVGPQEVPVPAMIASSDYYFSWLTWVTDERVCLQWLKRVQNVSVLSICDFREDWQTWDCPKTQEHIEESRTGWAGGFFVSTPVFSYDAISYYKIFSDKDGYKHIHYIKDTVENAIQITSGKWEAINIFRVTQDSLFYSSNEFEEYPGRRNIYRISIGSYPPSKKCVTCHLRKERCQYYTASFSDYAKYYALVCYGPGIPISTLHDGRTDQEIKILEENKELENALKNIQLPKEEIKKLEVDEITLWYKMILPPQFDRSKKYPLLIQVYGGPCSQSVRSVFAVNWISYLASKEGMVIALVDGRGTAFQGDKLLYAVYRKLGVYEVEDQITAVRKFIEMGFIDEKRIAIWGWSYGGYVSSLALASGTGLFKCGIAVAPVSSWEYYASVYTERFMGLPTKDDNLEHYKNSTVMARAEYFRNVDYLLIHGTADDNVHFQNSAQIAKALVNAQVDFQAMWYSDQNHGLSGLSTNHLYTHMTHFLKQCFSLSD

SEQ ID NO: 34 - protein, 159 aa, Artificial Sequence
  Synthetic Polypeptide
GSDLGKKLLEAARAGQDDEVRELLKAGADVNAKDVLGWTPLHLAAFEGHLEIVEVLLKAGADVNAKDKKGWTPLQLAARTGHLEIVEVLLKAGADVNAKDHIGATPLHLAAWQGHPEIVEVLLKAGADVNAQDKSGKTPADLAADAGHEDIAEVLQKAA

SEQ ID NO: 35 - protein, 126 aa, Artificial Sequence
  Synthetic Polypeptide
GSDLGQKLLKAAQEGQDDEVRELLKAGADVNAKDLRGITPLHVAAWQGHLEIVEVLLKAGADVNAKDSKGETPLHLAAFRGHLEIVEVLLKAGADVNAQDQQGETPADLAALAGHVDIAEVLQKAA

SEQ ID NO: 36 - protein, 124 aa, Artificial Sequence
  Synthetic Polypeptide
DLGKKLLEAARAGQDDEVRELLKAGADVNAKDYFSHTPLHLAARNGHLKIVEVLLKAGADVNAKDFAGKTPLHLAAADGHLEIVEVLLKAGADVNAQDIFGKTPADIAADAGHEDIAEVLQKLN

SEQ ID NO: 37 - protein, 728 aa, Artificial Sequence
  Synthetic Polypeptide
MRGSHHHHHHGSDLGKKLLEAARAGQDDEVRELLKAGADVNAKDYFSHTPLHLAARNGHLKIVEVLLKAGADVNAKDFAGKTPLHLAAADGHLEIVEVLLKAGADVNAQDIFGKTPADIAADAGHEDIAEVLQKAAGSPTPTPTTPTPTPTTPTPTPTGSDLGEKLLVAALYGQDDEVRELLKAGADVNAKDQWGLTPLHKAALQGHLEIVEVLLKAGADVNAKDERGHTPLHWAARFGHLEIVEVLLKAGADVNAQDQKGYTPADLAALWGHEDIAEVLQKAAGSPTPTPTTPTPTPTTPTPTPTGSDLGQKLLKAAQEGQDDEVRELLKAGADVNAKDLRGITPLHVAAWQGHLEIVEVLLKAGADVNAKDSKGETPLHLAAFRGHLEIVEVLLKAGADVNAQDQQGETPADLAALAGHVDIAEVLQKAAGSPTPTPTTPTPTPTTPTPTPTGSDLGQKLLKAAQEGQDDEVRELLKAGADVNAKDLRGITPLHVAAWQGHLEIVEVLLKAGADVNAKDSKGETPLHLAAFRGHLEIVEVLLKAGADVNAQDQQGETPADLAALAGHVDIAEVLQKAAGSPTPTPTTPTPTPTTPTPTPTGSDLGKKLLEAARAGQDDEVRELLKAGADVNAKDYFSHTPLHLAARNGHLKIVEVLLKAGADVNAKDFAGKTPLHLAAADGHLEIVEVLLKAGADVNAQDIFGKTPADIAADAGHEDIAEVLQKAA

SEQ ID NO: 38 - protein, 728 aa, Artificial Sequence
  Synthetic Polypeptide
MRGSHHHHHHGSDLGKKLLEAARAGQDDEVRELLKAGADVNAKDYFSHTPLHLAARNGHLKIVEVLLKAGADVNAKDFAGKTPLHLAAADGHLEIVEVLLKAGADVNAQDIFGKTPADIAADAGHEDIAEVLQKAAGSPTPTPTTPTPTPTTPTPTPTGSDLGKKLLEAARAGQDDEVRELLKAGADVNAKDKDGYTPLHLAAREGHLEIVEVLLKAGADVNAKDKDGYTPLHLAAREGHLEIVEVLLKAGADVNAQDKSGKTPADLAADAGHEDIAEVLQKAAGSPTPTPTTPTPTPTTPTPTPTGSDLGQKLLKAAQEGQDDEVRELLKAGADVNAKDLRGITPLHVAAWQGHLEIVEVLLKAGADVNAKDSKGETPLHLAAFRGHLEIVEVLLKAGADVNAQDQQGETPADLAALAGHVDIAEVLQKAAGSPTPTPTTPTPTPTTPTPTPTGSDLGQKLLKAAQEGQDDEVRELLKAGADVNAKDLRGITPLHVAAWQGHLEIVEVLLKAGADVNAKDSKGETPLHLAAFRGHLEIVEVLLKAGADVNAQDQQGETPADLAALAGHVDIAEVLQKAAGSPTPTPTTPTPTPTTPTPTPTGSDLGKKLLEAARAGQDDEVRELLKAGADVNAKDYFSHTPLHLAARNGHLKIVEVLLKAGADVNAKDFAGKTPLHLAAADGHLEIVEVLLKAGADVNAQDIFGKTPADIAADAGHEDIAEVLQKAA

SEQ ID NO: 39 - protein, 157 aa, Artificial Sequence
  Synthetic Polypeptide
DLGKKLLEAARAGQDDEVRILLAAGADVNAKDVLGWTPLHLAAFEGHLEIVEVLLKAGADVNAKDKKGWTPLQLAARTGHLEIVEVLLKAGADVNAKDHIGATPLHLAAWQGHPEIVEVLLKAGADVNAQDKSGKTPADLAADAGHEDIAEVLQKLN

SEQ ID NO: 40 - protein, 124 aa, Artificial Sequence
  Synthetic Polypeptide
DLGEKLLVAALYGQDDEVRILLAAGADVNAKDQWGLTPLHKAALQGHLEIVEVLLKAGADVNAKDERGHTPLHWAARFGHLEIVEVLLKAGADVNAQDQKGYTPADLAALWGHEDIAEVLQKLN

SEQ ID NO: 41 - protein, 157 aa, Artificial Sequence
  Synthetic Polypeptide
DLGKKLLEAARAGQDDEVRELLKAGADVNAKDEVGRTPLHLAAQIGHLEIVEVLLKAGADVNAKNRWGVTPLHVAAWVGHLEIVEVLLKAGADVNAKDQQGATPLHLAAIRGHLEIVEVLLKAGADVNAQDKSGKTPADLAADAGHEDIAEVLQKLN

SEQ ID NO: 42 - protein, 157 aa, Artificial Sequence
  Synthetic Polypeptide
DLGKKLLEAARAGQDDEVRELLKAGADVNAKDEWGKTPLHLAAIRGHLEIVEVLLKAGADVNAKDTHGITPLHLAAFVGHLEIVEVLLKAGADVNAKDYLGRTPLHIAAAKGHLEIVEVLLKAGADVNAQDKSGKTPADLAADAGHEDIVEVLQKLN

SEQ ID NO: 43 - protein, 124 aa, Artificial Sequence
  Synthetic Polypeptide
DLGEKLLVAALYGQDDEVRELLKAGADVNAKDQWGLTPLHKAALQGHLEIVEVLLKAGADVNAKDERGHTPLHWAARFGHLEIVEVLLKAGADVNAQDQKGYTPADLAALWGHEDIAEVLQKAA

SEQ ID NO: 44 - protein, 274 aa, Artificial Sequence
  Synthetic Polypeptide
GSDLGEKLLVAALYGQDDEVRILLAAGADVNAKDQWGLTPLHKAALQGHLEIVEVLLKAGADVNAKDERGHTPLHWAARFGHLEIVEVLLKAGADVNAQDQKGYTPADLAALWGHEDIAEVLQKAAGSPTPTPTTPTPTPTTPTPTPTGSDLGQKLLKAAQEGQDDEVRELLKAGADVNAKDLRGITPLHVAAWQGHLEIVEVLLKAGADVNAKDSKGETPLHLAAFRGHLEIVEVLLKAGADVNAQDQQGETPADLAALAGHVDIAEVLQKLN

SEQ ID NO: 45 - protein, 274 aa, Artificial Sequence
  Synthetic Polypeptide
GSDLGEKLLVAALYGQDDEVRILLAAGADVNAKDQWGLTPLHKAALQGHLEIVEVLLKAGADVNAKDERGHTPLHWAARFGHLEIVEVLLKAGADVNAQDQKGYTPADLAALWGHEDIAEVLQKAAGSPTPTPTTPTPTPTTPTPTPTGSDLGDKLLHAAYYGQDDEVRILLAAGADVNAKDTQGLTPLHLAAAIGHLEIVEVLLKAGADVNAKDAYGETPLHLVASRGHLEIVEVLLKAGADVNAQDYSGRTPADLAAEYGHEDIAEVLQKLN

SEQ ID NO: 46 - protein, 274 aa, Artificial Sequence
  Synthetic Polypeptide
GSDLGEKLLVAALYGQDDEVRILLAAGADVNAKDQWGLTPLHKAALQGHLEIVEVLLKAGADVNAKDERGHTPLHWAARFGHLEIVEVLLKAGADVNAQDQKGYTPADLAALWGHEDIAEVLQKAAGSPTPTPTTPTPTPTTPTPTPTGSDLGRKLLQAAQLGQDDEVRELLKAGADVNAKDSRGITPLHVAAWQGHLEIVEVLLKASADVNAKDHAGITPLHLAAFLGHLEIVEVLLKAGADVNAQDQEGRTPADLAALQGHEDIAKVLQKLN

SEQ ID NO: 47 - protein, 307 aa, Artificial Sequence
  Synthetic Polypeptide
GSDLGEKLLVAALYGQDDEVRILLAAGADVNAKDQWGLTPLHKAALQGHLEIVEVLLKAGADVNAKDERGHTPLHWAARFGHLEIVEVLLKAGADVNAQDQKGYTPADLAALWGHEDIAEVLQKAAGSPTPTPTTPTPTPTTPTPTPTGSDLGKKLLEAARAGQDDEVRELLKAGADVNAKDTYGETPLHLAAWKGHLEIVEVLLKAGADVNAKDSIGITPLHVAAHHGHLEIVEVLLKAGADVNAKDKWGRTPLHLAALLGHLEIAEVLLKAGADVNAQDKSGKTPADLAADAGHDDIAEVLQKLN

SEQ ID NO: 48 - protein, 308 aa, Artificial Sequence
  Synthetic Polypeptide
GSDLGEKLLVAALYGQDDEVRILLAAGADVNAKDQWGLTPLHKAALQGHLEIVEVLLKAGADVNAKDERGHTPLHWAARFGHLEIVEVLLKAGADVNAQDQKGYTPADLAALWGHEDIAEVLQKAAGSPTPTPTTPTPTPTTPTPTPTGSDLGKKLLEAARAGQDDEVRELLKAGADVNAKDQEHGSTPLHLAANYGHLEIVEVLLKAGADVNAKNWWGNTPLHLAAVYGHLEIVEVLLKAGADVNAKDNHGHTPLHLAAWYGHLEIVEVLLKAGADVNAQDKSGKTPADLAADAGHEDIAEVLQKAA

SEQ ID NO: 49 - protein, 274 aa, Artificial Sequence
  Synthetic Polypeptide
GSDLGEKLLVAALYGQDDEVRILLAAGADVNAKDQWGLTPLHKAALQGHLEIVEVLLKAGADVNAKDERGHTPLHWAARFGHLEIVEVLLKAGADVNAQDQKGYTPADLAALWGHEDIAEVLQKAAGSPTPTPTTPTPTPTTPTPTPTGSDLGKKLLVAANVGQDDEVRILLAAGADVNAKDYHGYTPLHHAATYGHLEIVEVLLKAGADVNAKDQTGLTPLHLAAAKGHLEIVEVLLKAGADVNAQDHHGYTPADLAAFVGHEDIAVVLQKLN

SEQ ID NO: 50 - protein, 274 aa, Artificial Sequence
  Synthetic Polypeptide
GSDLGEKLLVAALYGQDDEVRILLAAGADVNAKDQWGLTPLHKAALQGHLEIVEVLLKAGADVNAKDERGHTPLHWAARFGHLEIVEVLLKAGADVNAQDQKGYTPADLAALWGHEDIAEVLQKAAGSPTPTPTTPTPTPTTPTPTPTGSDLGKKLLEAARAGQDDEVRELLKAGADVNAKDKDGYTPLHLAAREGHLEIVEVLLKAGADVNAKDKDGYTPLHLAAREGHLEIVEVLLKAGADVNAQDKSGKTPADLAADAGHEDIAEVLQKAA

SEQ ID NO: 51 - protein, 124 aa, Artificial Sequence
  Synthetic Polypeptide
DLGKKLLVAANVGQDDEVRILLAAGADVNAKDYHGYTPLHHAATYGHLEIVEVLLKAGADVNAKDQTGLTPLHLAAAKGHLEIVEVLLKAGADVNAQDHHGYTPADLAAFVGHEDIAVVLQKLN

SEQ ID NO: 52 - protein, 124 aa, Artificial Sequence
  Synthetic Polypeptide
DLGRKLLQAAQLGQDDEVRELLKAGADVNAKDSRGITPLHVAAWQGHLEIVEVLLKASADVNAKDHAGITPLHLAAFLGHLEIVEVLLKAGADVNAQDQEGRTPADLAALQGHEDIAKVLQKLN

SEQ ID NO: 53 - protein, 157 aa, Artificial Sequence
  Synthetic Polypeptide
DLGKKLLEAARAGQDDEVRELLKAGADVNAKDTYGETPLHLAAWKGHLEIVEVLLKAGADVNAKDSIGITPLHVAAHHGHLEIVEVLLKAGADVNAKDKWGRTPLHLAALLGHLEIAEVLLKAGADVNAQDKSGKTPADLAADAGHDDIAEVLQKLN

SEQ ID NO: 54 - protein, 158 aa, Artificial Sequence
  Synthetic Polypeptide
DLGKKLLEAARAGQDDEVRELLKAGADVNAKDQEHGSTPLHLAANYGHLEIVEVLLKAGADVNAKNWWGNTPLHLAAVYGHLEIVEVLLKAGADVNAKDNHGHTPLHLAAWYGHLEIVEVLLKAGADVNAQDKSGKTPADLAADAGHEDIAEVLQKAA

SEQ ID NO: 55 - protein, 124 aa, Artificial Sequence; identical to SEQ ID NO: 51
  Synthetic Polypeptide

SEQ ID NO: 56 - protein, 10 aa, Artificial Sequence
  Synthetic Polypeptide
MRGSHHHHHH

SEQ ID NO: 57 - protein, 126 aa, Artificial Sequence
  Synthetic Polypeptide
GSDLGKKLLEAARAGQDDEVRELLKAGADVNAKDKDGYTPLHLAAREGHLEIVEVLLKAGADVNAKDKDGYTPLHLAAREGHLEIVEVLLKAGADVNAQDKSGKTPADLAADAGHEDIAEVLQKAA

(19) United States
(12) Patent Application Publication
(10) Pub. No.: US 2020/0385488 A1
(43) Pub. Date: Dec. 10, 2020

(54) MULTISPECIFIC PROTEINS

(71) Applicant: Molecular Partners AG, Zurich-Schlieren (CH)

(72) Inventors: Christian Reichen, Zurich-Schlieren (CH); Alexander Link, Zurich-Schlieren (CH); Julia Hepp, Zurich-Schlieren (CH); Victor Levitsky, Zurich-Schlieren (CH)

(21) Appl. No.: 16/891,249

(22) Filed: Jun. 3, 2020

Related U.S. Application Data
(60) Provisional application No. 62/857,037, filed on Jun. 4, 2019.

(51) Int. Cl. C07K 16/40; C07K 16/28

(52) U.S. Cl. CPC C07K 16/40; C07K 16/2878; C07K 2317/92; C07K 2317/31; C07K 2317/51; C07K 2317/94

(57) ABSTRACT

This disclosure relates to multispecific recombinant proteins useful for the treatment of cancer.

### CROSS-REFERENCE TO RELATED APPLICATION

The present application claims the benefit of priority to U.S. Provisional Application No. 62/857,037, filed Jun. 4, 2019, the disclosure of which is incorporated herein by reference in its entirety.

### FIELD OF THE INVENTION

The present invention relates to multispecific proteins useful for the treatment of cancer.

### INCORPORATION BY REFERENCE OF SEQUENCE LISTING

Incorporated by reference in its entirety is a computer-readable nucleotide/amino acid sequence listing submitted concurrently herewith and identified as follows: ASCII (text) file named 53893A_Seqlisting.txt; size: 107,894 bytes, created May 21, 2020.

### BACKGROUND OF THE INVENTION

Several members of the tumor necrosis factor receptor (TNFR) family function after initial T cell activation to sustain T cell responses and thus have pivotal roles in the organization and function of the immune system. CD27, 4-1BB (CD137), OX40 (CD134), HVEM, CD30, and GITR can have costimulatory effects on T cells, meaning that they sustain T-cell responses after initial T cell activation (Watts T. H. (2005) Annu. Rev. Immunol. 23, 23-68). Depending on the disease condition, stimulation via costimulatory TNF family members can exacerbate or ameliorate disease.

4-1BB (CD137), a member of the TNF receptor superfamily, has been first identified as a molecule whose expression is induced by T-cell activation (Kwon Y. H. and Weissman S. M. (1989), Proc. Natl. Acad. Sci. USA 86, 1963-1967). Subsequent studies demonstrated expression of 4-1BB in T- and B-lymphocytes, NK-cells, NKT-cells, monocytes, neutrophils, and dendritic cells as well as cells of non-hematopoietic origin such as endothelial and smooth muscle cells. Expression of 4-1BB in different cell types is mostly inducible and driven by various stimulatory signals, such as T-cell receptor (TCR) or B-cell receptor triggering, as well as signaling induced through co-stimulatory molecules or receptors of pro-inflammatory cytokines.

4-1BB signaling is known to stimulate IFNγ secretion and proliferation of NK cells, as well as to promote dendritic cell (DC) activation as indicated by their increased survival and capacity to secret cytokines and upregulate co-stimulatory molecules. However, 4-1BB is best characterized as a co-stimulatory molecule which modulates TCR-induced activation in both the CD4+ and CD8+ subsets of T-cells. In combination with TCR triggering, agonistic 4-1BB-specific antibodies enhance proliferation of T-cells, stimulate lymphokine secretion and decrease sensitivity of T-lymphocytes to activation-induced cells death (Snell L. M. et al. (2011) Immunol. Rev. 244, 197-217). In line with these co-stimulatory effects of 4-1BB antibodies on T-cells in vitro, their administration to tumor bearing mice leads to potent anti-tumor effects in many experimental tumor models (Melero I. et al. (1997), Nat. Med. 3, 682-685; Narazaki H. et al. (2010), Blood 115, 1941-1948). In vivo depletion experiments demonstrated that CD8+ T-cells play the most critical role in anti-tumoral effect of 4-1BB-specific antibodies. However, depending on the tumor model or combination therapy, which includes 4-1BB-specific antibodies, contributions of other types of cells such as DCs, NK-cells or CD4+ T-cells have been reported (MuriUo O. et al. (2009), Eur. J. Immunol. 39, 2424-2436; Stagg J. et al. (2011), Proc. Natl. Acad. Sci. USA 108, 7142-7147).

In addition to their direct effects on different lymphocyte subsets, 4-1BB agonists can also induce infiltration and retention of activated T-cells in the tumor through 4-1BB-mediated upregulation of intercellular adhesion molecule 1 (ICAM1) and vascular cell adhesion molecule 1 (VCAM1) on tumor vascular endothelium. 4-1BB triggering may also reverse the state of T-cell anergy induced by exposure to soluble antigen that may contribute to disruption of immunological tolerance in the tumor micro-environment or during chronic infections.

It has been reported that systemic administration of 4-1BB-specific agonistic antibodies induces expansion of CD8+ T-cells associated with liver toxicity (Dubrot J. et al. (2010), Cancer Immunol. Immunother. 59, 1223-1233). In human clinical trials (ClinicalTrials.gov, NCT00309023), 4-1BB agonistic antibodies (BMS-663513) administered once every three weeks for 12 weeks induced stabilization of the disease in patients with melanoma, ovarian or renal cell carcinoma. However, the same antibody given in another trial (NCT00612664) caused grade 4 hepatitis leading to termination of the trial (Simeone E. and Ascierto P. A. (2012), J. Immunotoxicology 9, 241-247).

Thus, there is a need for new generation agonists that effectively engage 4-1BB while avoiding undesired side effects.

### SUMMARY OF THE INVENTION

Based on the disclosure provided herein, those skilled in the art will recognize, or be able to ascertain using no more than routine experimentation, many equivalents to the specific embodiments of the invention described herein. Such equivalents are intended to be encompassed by the following embodiments (E).

E1. A recombinant protein comprising a first ankyrin repeat domain that specifically binds fibroblast activation protein (FAP) and a second ankyrin repeat domain that specifically binds 4-1BB.
E2. The recombinant protein of E1, further comprising a third ankyrin repeat domain that specifically binds 4-1BB.
E3. The recombinant protein of E2, wherein said ankyrin repeat domains are arranged, from the N-terminus to C-terminus, according to the following formula: (FAP-binding domain)-(4-1BB binding domain)-(4-1BB binding domain).
E4 The recombinant protein of any one of E1-E3, further comprising a half-life extending moiety.
E5. The recombinant protein of E4, wherein said half-life extending moiety comprises a fourth ankyrin repeat domain that specifically binds serum albumin.
E6. The recombinant protein of E5, further comprising a fifth ankyrin repeat domain that specifically binds serum albumin.
E7. The recombinant protein of E6, wherein said ankyrin repeat domains are arranged, from the N-terminus to C-terminus, according to the following formula: (serum albumin binding domain (also referenced herein as serum albumin binding domain 1))-(FAP-binding domain)-(4-1BB binding domain)-(4-1BB binding domain)-(serum albumin binding domain (also referenced herein as serum albumin binding domain 2)).
E8. The recombinant protein of any one of E1-E7, further comprising a linker between any of said FAP-binding domain, said 4-1BB binding domain, and said half-life extending moiety.
E9. The recombinant protein of any one of E1-E8, comprising the following formula, from the N-terminus to C-terminus: (FAP-binding domain)-(linker)-(4-1BB binding domain)-(linker)-(4-1BB binding domain).
E10. The recombinant protein of any one of E1-E8, comprising the following formula, from the N-terminus to C-terminus: (serum albumin binding domain)-(linker)-(FAP-binding domain)-(linker)-(4-1BB binding domain)-(linker)-(4-1BB binding domain)-(linker)-(serum albumin binding domain).
E11. The recombinant protein of any one of E4 or E8-E9, wherein said half-life extending moiety comprises an immunoglobulin heavy chain constant domain.
E12. The recombinant protein of E11, wherein said immunoglobulin domain is an Fc domain of an IgA1, IgA2, IgD, IgE, IgM, IgG1, IgG2, IgG3, or IgG4 immunoglobulin.
E13. The recombinant protein of E12, wherein said Fc domain is the Fc domain of human IgG1 immunoglobulin.
E14. The recombinant protein of E13, wherein said Fc domain comprises a modification to reduce the effector function.
E15. The recombinant protein of any one of E1-E14, wherein said FAP is human FAP.
E16. The recombinant protein of any one of E1-E15, wherein said 4-1BB is human 4-1BB.
E17. The recombinant protein of any one of E5-E10, wherein said serum albumin is human serum albumin (HSA).
E18. The recombinant protein of any one of E11-E14, wherein said immunoglobulin heavy chain constant domain is a human immunoglobulin heavy chain constant domain.
E19. The recombinant protein of any one of E1-E18, wherein binding of said recombinant protein to FAP does not reduce the protease activity of FAP by more than 25%, more than 20%, more than 15%, more than 10%, or more than 5%.
E20. The recombinant protein of any one of E1-E19, wherein said FAP-binding domain comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 2, and wherein optionally A at the second last position of SEQ ID NO: 2 is substituted with L and/or A at the last position of SEQ ID NO: 2 is substituted with N.
E21. The recombinant protein of any one of E1-E20, wherein said FAP-binding domain comprises the amino acid sequence of SEQ ID NO: 2.
E22. The recombinant protein of any one of E1-E19, wherein said FAP-binding domain comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to any one of SEQ ID NOs: 18-23 and 39-43.
E23. The recombinant protein of any one of E1-E20 and E22, wherein said FAP-binding domain comprises the amino acid sequence of any one of SEQ ID NOs: 18-23 and 39-43.
E24. The recombinant protein of any one of E1-E20 and E22, wherein said FAP-binding domain comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to any one of SEQ ID NOs: 2, 18-22, and 43, and wherein optionally A at the second last position is substituted with L, and/or A at the last position is substituted with N.
E25. The recombinant protein of any one of E1-E20 and E22, wherein said FAP-binding domain comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to any one of SEQ ID NOs:23 and 39-42, and wherein optionally L at the second last position is substituted with A, and/or N at the last position is substituted with A.
E26. The recombinant protein of any one of E1-E20 and E22, wherein said FAP-binding domain comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 39, and wherein optionally L at the second last position is substituted with A, and/or N at the last position is substituted with A.
E27. The recombinant protein of any one of E1-E20 and E22, wherein said FAP-binding domain comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 43, and wherein optionally A at the second last position is substituted with L, and/or A at the last position is substituted with N.
E28. The recombinant protein of any one of E1-E27, wherein said FAP-binding domain (i) comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to any one of SEQ ID NOs: 2, 18-23, and 39-43, and (ii) further comprises at its N-terminus, a G, an S, or a GS.
E29. The recombinant protein of any one of E1-E27, wherein said FAP-binding domain comprises an amino acid sequence that is at least 90% identical to any one of SEQ ID NOs: 2, 18-23, and 39-43, and further comprises at its N-terminus, a G, an S, or a GS.
E30. The recombinant protein of any one of E1-E29, wherein said 4-1BB binding domain or each of said 4-1BB binding domains independently comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 3, and wherein optionally A at the second last position of SEQ ID NO: 3 is substituted with L, and/or A at the last position of SEQ ID NO: 3 is substituted with N.
E31. The recombinant protein of any one of E1-E30, wherein said 4-1BB binding domain or each of said 4-1BB binding domains comprises the amino acid sequence of SEQ ID NO: 3.
E32. The recombinant protein of any one of E1-E29, wherein said 4-1BB-binding domain or each of said 4-1BB binding domains independently comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to any one of SEQ ID NOs: 24-29 and 51-55.
E33. The recombinant protein of any one of E1-E30 and E32, wherein said 4-1BB-binding domain or each of said 4-1BB binding domains comprises the amino acid sequence of any one of SEQ ID NOs: 24-29 and 51-55.
E34. The recombinant protein of any one of E1-E30 and E32, wherein said 4-1BB binding domain or each of said 4-1BB binding domains independently comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to any one of SEQ ID NOs: 3, 24-28, and 54, and wherein optionally A at the second last position is substituted with L, and/or A at the last position is substituted with N.
E35. The recombinant protein of any one of E1-E30 and E32, wherein said 4-1BB binding domain or each of said 4-1BB binding domains independently comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 54, and wherein optionally A at the second last position is substituted with L, and/or A at the last position is substituted with N.
E36. The recombinant protein of any one of E1-E30 and E32, wherein said 4-1BB binding domain or each of said 4-1BB binding domains independently comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to any one of SEQ ID NOs: 29, 51-53, and 55, and wherein optionally L at the second last position is substituted with A, and/or N at the last position is substituted with A.
E37. The recombinant protein of any one E1-E36, wherein said 4-1BB binding domain or each of said 4-1BB binding domains independently (i) comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to any one of SEQ ID NOs: 3, 18-29, and 51-55, and (ii) further comprises at its N-terminus, a G, an S, or a GS.
E38. The recombinant protein of any one E1-E37, wherein said 4-1BB binding domain or each of said 4-1BB binding domains independently comprises an amino acid sequence that is at least 90% identical to any one of SEQ ID NOs: 3, 18-29, and 51-55, and further comprises at its N-terminus, a G, an S, or a GS.
E39. The recombinant protein of any one of E1-E38, wherein:
(a) said FAP binding domain comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to any one of SEQ ID NOs: 2, 18-23, and 39-43; wherein its N-terminus optionally further comprises a G, an S, or a GS; and wherein the second last position can be L or A, and the last position can be N or A; and
(b) said 4-1BB binding domain or each of said 4-1BB binding domains independently comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to any one of SEQ ID NOs: 3, 24-29, and 51-55; wherein its N-terminus optionally further comprises a G, an S, or a GS; and wherein the second last position can be L or A, and the last position can be N or A.
E40. The recombinant protein of any one of E1-E35, wherein:
(a) said FAP binding domain comprises an amino acid sequence that is at least 90% identical to any one of SEQ ID NOs: 2, 18-23, and 39-43; wherein its N-terminus optionally further comprises a G, an S, or a GS; and wherein the second last position can be L or A, and the last position can be N or A; and
(b) said 4-1BB binding domain or each of said 4-1BB binding domains independently comprises an amino acid sequence that is at least 90% identical to any one of SEQ ID NOs: 3, 24-29, and 51-55; wherein its N-terminus optionally further comprises a G, an S, or a GS; and wherein the second last position can be L or A, and the last position can be N or A.
E41. The recombinant protein of any one of E1-E34, wherein:
(a) said FAP binding domain comprises an amino acid sequence that is at least 95% identical to any one of SEQ ID NOs: 2, 18-23, and 39-43; wherein its N-terminus optionally further comprises a G, an S, or a GS; and wherein the second last position can be L or A, and the last position can be N or A; and
(b) said 4-1BB binding domain or each of said 4-1BB binding domains independently comprises an amino acid sequence that is at least 95% identical to any one of SEQ ID NOs: 3, 24-29, and 51-55; wherein its N-terminus optionally further comprises a G, an S, or a GS; and wherein the second last position can be L or A, and the last position can be N or A.
E42. The recombinant protein of any one of E1-E34, wherein:
(a) said FAP binding domain comprises an amino acid sequence of any one of SEQ ID NOs: 2, 18-23, and 39-43; wherein its N-terminus optionally further comprises a G, an S, or a GS; and wherein the second last position can be L or A, and the last position can be N or A; and
(b) said 4-1BB binding domain or each of said 4-1BB binding domains independently comprises an amino acid sequence of any one of SEQ ID NOs: 3, 24-29, and 51-55; wherein its N-terminus optionally further comprises a G, an S, or a GS; and wherein the second last position can be L or A, and the last position can be N or A.
E43. The recombinant protein of any one of E5-E10, E15-E17, and E19-E42, wherein said serum albumin binding domain or each of said serum albumin binding domains independently comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 1, and wherein optionally A at the second last position is substituted with L, and/or A at the last position is substituted with N.
E44. The recombinant protein of any one of E5-E10, E15-E17, and E19-E43, wherein said serum albumin binding domain or each of said serum albumin binding domains independently comprises the amino acid sequence of SEQ ID NO: 1.
E45. The recombinant protein of any one of E5-E10, E15-E17, and E19-E42, wherein said serum albumin binding domain or each of said serum albumin binding domains independently comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to any one of SEQ ID NOs: 30-31, and wherein optionally A at the second last position of any one of SEQ ID NOs: 30-31 is substituted with L, and/or A at the last position of any one of SEQ ID NOs: 30-31 is substituted with N.
E46. The recombinant protein of any one of E5-E10, E15-E17, E19-E42, and E45, wherein said serum albumin binding domain or each of said serum albumin binding domains independently comprises the amino acid sequence of any one of SEQ ID NOs: 30-31.
E47. The recombinant protein of any one of E5-E10, E15-E17, and E19-E46, wherein said serum albumin binding domain or each of said serum albumin binding domains independently (i) comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to any one of SEQ ID NOs: 1 and 30-31, and (ii) further comprises at its N-terminus, a G, an S, or a GS.
E48. The recombinant protein of any one of E5-E10, E15-E17, and E19-E47, wherein said serum albumin binding domain or each of said serum albumin binding domains independently comprises an amino acid sequence that is at least 90% identical to any one of SEQ ID NOs: 1 and 30-31, and further comprises at its N-terminus, a G, an S, or a GS.
E49. The recombinant protein of any one of E7-E10, E15-E17, and E19-E48, wherein the N-terminal serum albumin domain (or serum albumin domain 1) comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 5, and wherein optionally A at the second last position is substituted with L, and/or A at the last position is substituted with N.
E50. The recombinant protein of any one of E8-E49, wherein said linker comprises the amino acid sequence of SEQ ID NO: 4.
E51. A recombinant protein comprising an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 6, wherein said protein specifically binds FAP and 4-1BB.
E52. The recombinant protein of E51, wherein said FAP is human FAP.
E53. The recombinant protein of E51 and E52, wherein said 4-1BB is human 4-1BB.
E54. The recombinant protein of any one of E1-E53, wherein said recombinant protein binds FAP with a KD value of less than or equal to: about 50 nM, about 40 nM, about 30 nM, about 20 nM, about 10 nM, about 5 nM, about 2 nM, about 1 nM, about 900 pM, about 800 pM, about 700 pM, about 600 pM, about 500 pM, about 400 pM, about 300 pM, about 250 pM, about 200 pM, about 150 pM, about 100 pM, about 50 pM, about 40 pM, about 30 pM, about 25 pM, about 20 pM, about 15 pM, about 10 pM, about 5 pM, or about 1 pM.
E55. The recombinant protein of any one of E1-E54, wherein said recombinant protein binds human FAP with a KD value of less than or equal to about 10 nM.
E56. The recombinant protein of any one of E1-E54, wherein said recombinant protein binds human FAP with a KD value of less than or equal to about 1 nM.
E57. The recombinant protein of any one of E1-E56, wherein said recombinant protein binds 4-1BB with a KD value of less than or equal to: about 50 nM, about 40 nM, about 30 nM, about 20 nM, about 10 nM, about 5 nM, about 2 nM, about 1 nM, about 900 pM, about 800 pM, about 700 pM, about 600 pM, about 500 pM, about 400 pM, about 300 pM, about 250 pM, about 200 pM, about 150 pM, about 100 pM, about 50 pM, about 40 pM, about 30 pM, about 25 pM, about 20 pM, about 15 pM, about 10 pM, about 5 pM, or about 1 pM.
E58. The recombinant protein of any one of E1-E57, wherein said recombinant protein binds human 4-1BB with a KD value of less than or equal to 10 nM.
E59. The recombinant protein of any one of E1-E57, wherein said recombinant protein binds human 4-1BB with a KD value of less than or equal to 1 nM.
E60. The recombinant protein of any one of E1-E57, wherein said recombinant protein binds human 4-1BB with a KD value of less than or equal to 50 pM.
E61. The recombinant protein of any one of E54-E60, wherein said KD is measured in PBS by surface plasmon resonance (SPR).
E62. The recombinant protein of E61, wherein said KD is measured using a Biacore T200 instrument.
E63. The recombinant protein of any one of E54-E60, wherein said KD is measured by bio-layer interferometry (BLI).
E64. The recombinant protein of E63, wherein said KD is measured using a ForteBio Octet instrument.
E65. The recombinant protein of any one of E1-E64, wherein said recombinant protein has a half maximal effective concentration (EC₅₀) of no more than about 100 nM, no more than about 75 nM, no more than about 65 nM, no more than about 55 nM, no more than about 45 nM, no more than about 35 nM, no more than about 25 nM, no more than about 15 nM, no more than about 10 nM, no more than about 5 nM, no more than about 4 nM, no more than about 3 nM, no more than about 2 nM, from about 0.01 nM to about 50 nM, from about 0.01 nM to about 25 nM, from about 0.01 nM to about 10 nM, from about 0.01 nM to about 5 nM, from about 0.05 nM to about 50 nM, from about 0.05 nM to about 25 nM, from about 0.05 nM to about 10 nM, from about 0.05 nM to about 5 nM, from about 0.1 nM to about 50 nM, from about 0.1 nM to about 25 nM, from about 0.1 nM to about 10 nM, from about 0.1 nM to about 5 nM, from about 0.4 nM to about 2 nM, as assessed by an in vitro IFNγ release assay.
E66. The recombinant protein of any one of E1-E65, wherein said recombinant protein has an EC₅₀ of no more than about 10 nM.
E67. The recombinant protein of any one of E1-E66, wherein said recombinant protein has an EC₅₀ of from about 0.1 nM to about 10 nM.
E68. The recombinant protein of any one of E65-E67, wherein said IFNγ release assay is a human T cell IFNγ release assay.
E69. The recombinant protein of E68, wherein said T cell is CD8+ T cell.
E70. The recombinant protein of any one of E65-E69, wherein said IFNγ release assay is measured using Human IFN-gamma DuoSet ELISA (R&D systems).
E71. A recombinant protein comprising the amino acid sequence of SEQ ID NO: 6.
E72. An isolated nucleic acid molecule encoding the recombinant protein of any one of E1-E71.
E73. The isolated nucleic acid molecule of E72, comprising the nucleic acid sequence of SEQ ID NO: 17.
E74. A recombinant protein comprising an amino acid sequence encoded by the sequence of SEQ ID NO:17.
E75. A recombinant protein comprising an amino acid sequence encoded by a nucleic acid sequence that is at least 85%, 90%, 95%, or 99% identical to the sequence of SEQ ID NO: 17:
E76. A recombinant protein comprising an amino acid sequence encoded by a nucleic acid sequence capable of hybridizing under highly stringent conditions to the sequence of SEQ ID NO: 17.
E77. A vector comprising a nucleic acid molecule comprising the nucleic acid sequence as defined in any one of E72-E76.
E78. A host cell comprising the nucleic acid molecule comprising the nucleic acid sequence of as defined in any one of E72-E76.
E79. A host cell comprising the vector of E77.
E80. The host cell of E78 or E79, wherein said cell is a bacterial cell.
E81. The host cell of E78 or E79, wherein said host cell is E. coli.
E82. The host cell of E78 or E79, wherein said cell is a eukaryotic cell.
E83. A method of making the recombinant protein of any one of E1-E71 and E74-E76, comprising culturing the host cell of any one of E78-E82 under conditions wherein said recombinant protein is expressed.
E84. The method of E83, further comprising isolating said recombinant protein.
E85. A pharmaceutical composition comprising the recombinant protein of any one of E1-E71 and E74-E76, and a pharmaceutically acceptable carrier or excipient.
E86. A method of treating cancer, comprising administering to a subject in need thereof a therapeutically effective amount of the recombinant protein of any one of E1-E71 and E74-E76, or the pharmaceutical composition of E85.
E87. The method of E86, wherein said subject is a human.
E88. The method of E86 or E87, wherein said cancer comprises a solid tumor.
E89. The method of any one of E86-E88, wherein said cancer comprises cells that express FAP.
E90. The method of any one of E86-E89, wherein the cancer is brain cancer, bladder cancer, breast cancer, clear cell kidney cancer, cervical cancer, colon cancer, rectal cancer, endometrial cancer, gastric cancer, head/neck squamous cell carcinoma, lip cancer, oral cancer, liver cancer, lung squamous cell carcinoma, melanoma, mesothelioma, non-small-cell lung cancer (NSCLC), non-melanoma skin cancer, ovarian cancer, pancreatic cancer, prostate cancer, renal cell carcinoma, sarcoma, small-cell lung cancer (SCLC), Squamous Cell Carcinoma of the Head and Neck (SCCHN), triple negative breast cancer, or thyroid cancer.
E91. The method of any one of E86-E89, wherein the cancer is adrenocortical tumor, alveolar soft part sarcoma, carcinoma, chondrosarcoma, colorectal carcinoma, desmoid tumors, desmoplastic small round cell tumor, endocrine tumors, endodermal sinus tumor, epithelioid hemangioendothelioma, Ewing sarcoma, germ cell tumor, hepatoblastoma, hepatocellular carcinoma, melanoma, nephroma, neuroblastoma, non-rhabdomyosarcoma soft tissue sarcoma (NRSTS), osteosarcoma, paraspinal sarcoma, renal cell carcinoma, retinoblastoma, rhabdomyosarcoma, synovial sarcoma, or Wilms tumor.
E92 The method of E86 or E87, wherein the cancer is acute lymphoblastic leukemia (ALL), acute myeloid leukemia (AML), chronic lymphocytic leukemia (CLL), or chronic myeloid leukemia (CML).
E93. The method of E86 or E87, wherein the cancer is diffuse large B-cell lymphoma (DLBCL), follicular lymphoma, Hodgkin's lymphoma (HL), mantle cell lymphoma (MCL), multiple myeloma (MM), myelodysplastic syndrome (MDS), non-Hodgkin's lymphoma (NHL), or small lymphocytic lymphoma (SLL).
E94. The method of any one of E86-E93, wherein said recombinant protein or pharmaceutical composition is administered intravenously.
E95. The method of any one of E86-E93, wherein said recombinant protein or pharmaceutical composition is administered subcutaneously.
E96. The method of any one of E86-E95, wherein said recombinant protein or pharmaceutical composition is administered about twice a week, once a week, once every two weeks, once every three weeks, once every four weeks, once every five weeks, once every six weeks, once every seven weeks, once every eight weeks, once every nine weeks, once every ten weeks, twice a month, once a month, once every two months, once every three months, or once every four months.
E96a. The method of any one of E86-E95, wherein said recombinant protein or pharmaceutical composition is administered at a dose range of from about 0.5 mg/kg to about 5 mg/kg, or from about 0.015 mg/kg to about 12 mg/kg.
E96b. The method of any one of E86-E95, wherein said recombinant protein or pharmaceutical composition is administered at the dose of about 2 mg/kg.
E96c. The method of any one of E86-E95, wherein said recombinant protein or pharmaceutical composition is administered every three weeks.
E97. The recombinant protein of any one of E1-E71 and E74-E76, or the pharmaceutical composition of E85 for use as a medicament.
E98. The recombinant protein of any one of E1-E71 and E74-E76, or the pharmaceutical composition of E85 for use in treating cancer in a subject.
E99. Use of the recombinant protein of any one of E1-E71 and E74-E76, or the pharmaceutical composition of E85 in the manufacture of a medicament for treating cancer in a subject.
E100. Use of the recombinant protein of any one of E1-E71 and E74-E76, or the pharmaceutical composition of E85 for treating cancer in a subject.
E101. A kit comprising a container, a composition within the container comprising the recombinant protein of any one of E1-E71 and E74-E76, or the pharmaceutical composition of E85, and a package insert containing instructions to administer a therapeutically effective amount of the recombinant protein or the pharmaceutical composition for treatment of a patient in need thereof.

The use of section headings herein is merely for the convenience of reading, and not intended to be limiting per se. The entire document is intended to be viewed as a unified disclosure, and it should be understood that all combinations of features described herein are contemplated.

### BRIEF DESCRIPTION OF THE FIGURES

FIG. 1 is an illustration of a recombinant multispecific protein of the disclosure. A serum albumin binding ankyrin repeat domain is linked to a FAP binding ankyrin repeat domain, which is linked to a 4-1BB binding ankyrin repeat domain, which is linked to another 4-1BB ankyrin repeat domain, which is linked to a serum albumin binding ankyrin repeat domain, via a series of linkers.

FIG. 2 is an amino acid sequence of a multispecific recombinant protein of the disclosure having the formula: (serum albumin binding domain)-(linker)-(FAP-binding domain)-(linker)-(4-1BB binding domain)-(linker)-(4-1BB binding domain)-(linker)-(serum albumin binding domain). (SEQ ID NO: 6). The sequence of the serum albumin binding domains are underlined, the sequence of the FAP-binding domain is italicized, the sequence of the 4-1BB binding domains are bolded, and the linkers are shaded.

FIG. 3 is a schematic illustrating FAP/4-1BB bispecific protein-mediated clustering of 4-1BB on T cells in proximity to tumor cells, triggering an immune response. In the absence of the tumor antigen FAP (normal, non-malignant cells; see “periphery” on the right side), minimal clustering of 4-1BB will occur due to lack of FAP binding, and immune activation will be limited. In contrast, in cancer-associated fibroblasts (“Tumor” on the left side), FAP is highly expressed (shown as solid triangles); therefore, through FAP-binding, the bispecific molecule promotes 4-1BB clustering and T-cell co-stimulation.

FIG. 4 is a chart setting forth various sequences referenced herein.

FIGS. 5A-5G describe the design and selected functional data of six 4-1BB/FAP bispecific proteins (FIG. 5A) Design: genetic fusions of ankyrin repeat domain specifically binding to human FAP with various numbers of 4-1BB-specific ankyrin repeat domains. (FIGS. 5B-5G) In vitro 4-1BB reporter cell assay. Activation of the 4-1BB signaling pathway in human 4-1BB transfected HT1080 cells was measured by an NF-κB-Luciferase reporter assay in the presence of FAP-expressing cells. The luminescence signal was used as a relative measure of 4-1BB pathway activation.

FIG. 6 is a graph showing SPR trace of simultaneous binding of MpA to h4-1BB, hFAP and HSA. Line (a) Binding of MpA, or PBST, to immobilized h4-1BB. Line (b) Association of hFAP to the h4-1BB/MpA complex, or PBST control, respectively. Line (c) Binding of HSA to the h41 BB-MpA-hFAP complex, or the PBST control, respectively, followed by a 1000 s dissociation phase.

FIG. 7 is a graph demonstrating that MpA enhances IFNγ production by primary human T cells in vitro. Dose-dependent enhancement of IFNγ production by purified CD8 T cells stimulated with plate bound anti-CD3 antibody plus increasing concentrations of MpA and controls bound to plate-coated human FAP was measured by ELISA. MpA and anti-FAP-4-1BBL led to activation of CD8 T cells resulting in an increase in IFNγ secretion in a dose dependent manner when bound to the plate via coated FAP. The non-FAP-targeted control MpC did not enhance IFNγ production by the T cells.

FIG. 8 is a graph showing the group mean serum concentration-time profiles of MpA in BALB/c mice (mean+/−max/min, N=3 per group) following single intravenous bolus administration of 1 mg/kg.

FIG. 9 is a graph showing mean serum concentration-time profile of MpA in BALB/c mice (mean+/−max/min, N=6 at 168 hr time point, N=3 all other time points) following single intravenous bolus administration of 1 mg/kg.

FIG. 10 is a graph showing serum concentration-time profile (filled symbols) and ADA titer-time profile (open symbols) of MpA in cynomolgus monkey following single intravenous infusion of 0.1 mg/kg. First concentration value BLQ was set to 0.2 nmol/L (5× lower than LLOQ) to indicate the course of the trace. AMA-negative samples were blotted with a titer of 100 (=MRD) to indicate the course of the trace. AMA titer values determined in pre-dose samples are blotted at t=0 h.

FIG. 11 is a graph showing serum concentration-time profile (filled symbols) and ADA titer-time profile (open symbols) of MpA in cynomolgus monkey following single intravenous infusion of 1 mg/kg. First concentration value BLQ was set to 0.2 nmol/L (5× lower than LLOQ) to indicate the course of the trace. ADA-negative samples were blotted with a titer of 100 (=MRD) to indicate the course of the trace. ADA titer values determined in pre-dose samples are blotted at t=0 h.

FIG. 12 is a graph showing serum concentration-time profile (filled symbols) and AMA titer-time profile (open symbols) of MpA in cynomolgus monkey following single intravenous infusion of 10 mg/kg. First concentration value BLQ was set to 0.2 nmol/L (5× lower than LLOQ) to indicate the course of the trace. AMA-negative samples were blotted with a titer of 100 (=MRD) to indicate the course of the trace. AMA titer values determined in pre-dose samples are blotted at t=0 h.

FIG. 13 is a graph showing serum concentration-time profiles of MpA in cynomolgus monkeys following single intravenous infusion of 0.1, 1 and 10 mg/kg. First value BLQ was set to 0.2 nmol/L (5× lower than LLOQ) to indicate the course of the traces.

FIG. 14 is a graph showing dose-normalized serum concentration-time profiles of MpA in cynomolgus monkeys following single intravenous infusion of 0.1, 1 and 10 mg/kg. Values considered to be affected by ADAs were excluded.

FIG. 15 is a graph showing dose-normalized serum concentration-time profiles of MpA in cynomolgus monkeys following single intravenous infusion of 0.1, 1 and 10 mg/kg. Values considered to be affected by ADAs were excluded.

FIGS. 16A and 16B. Tumor growth in HT-29 xenograft tumor-bearing NOG mice engrafted with human PBMCs. Mice were treated with anti-h4-1BB mAb 20H4.9, anti-FAP-4-1BBL fusion protein or MpB, a mouse surrogate of MpA. FIG. 16A is a graph showing the mean tumor volumes in mice receiving MpB, anti-h4-1BB mAb 20H4.9, anti-FAP-4-1BBL fusion protein or vehicle control.

FIG. 16B includes graphs showing tumor volumes from individual mice over time (days).

FIG. 17. Administration of anti-h4-1BB mAb 20H4.9 but not MpB induced increased liver T cell infiltration by human PBMCs in NOG mice.

FIG. 18 shows average FAP activity in presence of various recombinant molecules (shown in Table 19). Recombinant human FAP (rhFAP) converted substrate Z-GLY-PRO-AMC into a fluorescence product which was measured after 45 minutes at 460 nm (normalized to 100% activity-1st sample). In comparison to the background activity (2nd and 3rd sample), molecules Nos. 1 and 3 (MpA, and “F” (which is the FAP-binding domain of MpA)) showed no inhibitory effect on the FAP peptidase activity, similar to negative controls that do not bind to FAP (negative controls MpC and “N”). Partial inhibition of FAP activity was observed for Ft (an alternative FAP-binding ankyrin repeat domain, used as control), or for a protease inhibitor mixture (PI), which showed a dose dependent inhibition (using 1×, 3×, 5× concentrated PI mixtures). No inhibition was observed for FAP-binding antibody. Average FAP activity (in %) are shown from quadruplet measurement after signal normalization as mean and standard deviation. Abbreviations: H=albumin binding domain; F=hFAP-binding domain; F†=Alternative hFAP-binding domain—showing FAP activity inhibition (control); B=h4-1BB binding domain; N=none-target binding domain (negative control).

FIGS. 19A and 19B summarize functional and pharmacokinetic comparisons of various multispecific proteins with different binding domain configurations. FIG. 19A shows results of an in vitro 4-1BB reporter cell assay. Activation of the 4-1BB signaling pathway in human 4-1BB transfected HT1080 cells was measured by an NF-κB-Luciferase reporter assay in the presence of FAP-expressing cells. The luminescence signal was used as a relative measure of 4-1BB pathway activation. The arrangement of binding domains in the various multispecific proteins from N-terminus to C-terminus is indicated. FIG. 19B summarizes the results of pharmacokinetics studies in mice. This graph shows the mean serum concentration-time profiles in BALB/c mice (mean+/−max/min, N=3 per group) following single intravenous bolus administration of 1 mg/kg. The arrangement of binding domains in the various multispecific proteins from N-terminus to C-terminus is indicated. H=HSA binding domain, F=FAP binding domain; B=4-1BB binding domain.

FIG. 20 predicts various PD markers versus dose in human. Exposure values (Cₐᵥ) derived from the established minimal PBPK models (based on Zhao, J., Y. Cao, and W. J. Jusko, Across-Species Scaling of Monoclonal Antibody Pharmacokinetics Using a Minimal PBPK Model. Pharm Res, 2015. 32(10): p. 3269-81.) were used to translate PD effects from the mouse tumor studies (all as % of max. effect) and predict the dose-effect relationships in humans. Prediction intervals (shaded areas) are based on lower and upper bounds set during scaling of clearance to humans. Note: Predicted systemic CD8 T cell activation and expansion was based on the humanized PBMC mouse model. No systemic T cell activation was observed in healthy NHP.

### DETAILED DESCRIPTION OF THE INVENTION

### 1. Overview

Disclosed herein is a recombinant protein comprising designed ankyrin repeat domains with binding specificity for FAP and 4-1BB. Also disclosed are nucleic acids encoding the binding protein, pharmaceutical compositions comprising the binding protein or nucleic acid, and methods of using the binding protein, nucleic acid, or pharmaceutical compositions. In one aspect, the materials and methods of the disclosure take advantage of FAP's expression in tumor-associated stroma, allowing, e.g., specific targeting of lymphocytes in the tumor and selective activation of 4-1BB in those lymphocytes.

4-1BB agonist antibodies have demonstrated efficacy in prophylactic and therapeutic settings in both monotherapy and combination therapy tumor models, and have established durable anti-tumor protective T-cell memory responses. However, clinical development of 4-1BB agonistic antibodies has been hampered by dose-limiting hepatotoxicity. For example, phase I and II data from Urelumab (BMS-663513) (U.S. Patent Appl. Pub. No. 2017/0247455 A1) revealed a liver toxicity that appeared to be on target and dose dependent, halting clinical development of Urelumab.

The multispecific recombinant proteins described herein promote cancer target-mediated clustering of 4-1BB, thereby addressing challenges associated with previous therapies (see, e.g., FIG. 3). 4-1BB undergoes trimerization upon binding to its ligand (4-1BBL); and 4-1BB multimerization and clustering is a prerequisite for activation of its signaling pathway. The multispecific recombinant proteins disclosed herein take advantage of this clustering effect; and the activation of 4-1BB is linked to the expression of tumor antigen fibroblast activation protein (FAP).

Fibroblast-activation protein a (FAP, also known as Seprase) is a type II membrane-bound glycoprotein abundantly expressed in the stroma of many solid tumors by cancer-associated fibroblasts. FAP is expressed selectively in reactive stromal fibroblasts of more than 90% of epithelial malignancies (primary and metastatic), including lung, colorectal, bladder, ovarian and breast carcinomas, and in malignant mesenchymal cells of bone and soft tissue sarcomas, while it is generally absent from normal adult tissues (Brennen et al., Mol Cancer Ther. 11: 257-266 (2012); Garin-Chesa et al., Proc Natl Acad Sci USA 87, 7235-7239 (1990); Rettig et al., Cancer Res. 53:3327-3335 (1993); Rettig et al., Proc Natl Acad Sci USA 85, 3110-3 114 (1988)). FAP is also expressed on certain malignant tumor cells.

Although not wishing to be bound by a particular theory, FIG. 3 illustrates examples of advantages of the multispecific molecule. In the absence of the tumor antigen FAP (normal, non-malignant cells), minimal clustering of 4-1BB will occur, and immune activation will be limited. In contrast, in cancer-associated fibroblasts, FAP is highly expressed; therefore, through FAP-binding, the multispecific molecule promotes 4-1BB clustering and T-cell co-stimulation. The advantages of this strategy are twofold: systemic toxicities should be limited because activation will be largely confined to tissue expressing FAP, and tumor-mediated 4-1BB clustering should drive potent agonism.

### 2. Definitions

Unless otherwise defined herein, scientific and technical terms used in connection with the present invention shall have the meanings that are commonly understood by those of ordinary skill in the art. Further, unless otherwise required by context, singular terms shall include pluralities and plural terms shall include the singular. Generally, nomenclatures used in connection with, and techniques of, cell and tissue culture, molecular biology, immunology, microbiology, genetics and protein and nucleic acid chemistry and hybridization described herein are those well-known and commonly used in the art.

The terms “comprising,” “having,” “including,” and “containing” are to be construed as open-ended terms unless otherwise noted. If aspects of the invention are described as “comprising” a feature, embodiments also are contemplated “consisting of” or “consisting essentially of” the feature. The use of any and all examples, or exemplary language (e.g., “such as”) provided herein, is intended merely to better illustrate the disclosure and does not pose a limitation on the scope of the disclosure unless otherwise claimed. No language in the specification should be construed as indicating any non-claimed element as essential to the practice of the disclosure. Other than in the operating examples, or where otherwise indicated, all numbers expressing quantities of ingredients or reaction conditions used herein should be understood as modified in all instances by the term “about” as that term would be interpreted by the person skilled in the relevant art.

Recitation of ranges of values herein are merely intended to serve as a shorthand method of referring individually to each separate value falling within the range and each endpoint, unless otherwise indicated herein, and each separate value and endpoint is incorporated into the specification as if it were individually recited herein.

“Ankyrin repeat domain” refers to a domain that comprises at least one ankyrin repeat motif, which is originally derived from the repeat units of naturally occurring ankyrin repeat proteins. In general, the ankyrin repeat motif comprises about 33 residues that form two alpha helices, separated by loops. Ankyrin repeat proteins are known in the art. See, for example, International Patent Publication Nos. WO 2002/020565, WO 2010/060748, WO 2011/135067, WO 2012/069654, WO 2012/069655, WO 2014/001442, WO 2014/191574, WO 2014/083208, WO 2016/156596, and WO 2018/054971, all of which are incorporated by reference in their entireties. Ankyrin repeat domains optionally further comprise appropriate capping modules.

Ankyrin repeat domains may be modularly assembled into larger ankyrin repeat proteins according to the present disclosure, optionally with half-life extension domains, using standard recombinant DNA technologies (see, e.g., Forrer, P., et al., FEBS letters 539, 2-6, 2003, WO2012/069655, WO 2002/020565).

An ankyrin repeat domain “specifically binds” or “preferentially binds” (used interchangeably herein) to a target if it reacts or associates more frequently, more rapidly, with greater duration and/or with greater affinity with a particular target (e.g., cell or substance) than it does with alternative targets (e.g., cells or substances). For example, an ankyrin repeat domain that specifically binds FAP is an ankyrin repeat domain that binds FAP with greater affinity, avidity, more readily, and/or with greater duration than it binds to other non-FAP proteins. It is also understood by reading this definition that, for example, an ankyrin repeat domain which specifically or preferentially binds to a first target may or may not specifically or preferentially bind to a second target. As such, “specific binding” does not necessarily require (although it can include) exclusive binding. In general, under designated assay conditions, an ankyrin repeat domain binds preferentially to a particular target molecule and does not bind in a significant amount to other components present in a test sample.

A variety of assay formats may be used to select or characterize an ankyrin repeat domain that specifically binds a molecule of interest. For example, solid-phase ELISA immunoassay, immunoprecipitation, BIAcore™ (GE Healthcare, Piscataway, N.J.), fluorescence-activated cell sorting (FACS), Octet™ (ForteBio, Inc., Menlo Park, Calif.) and Western blot analysis are among many assays that may be used to identify an ankyrin repeat domain that specifically reacts with a target. Typically, a specific or selective reaction will be at least twice background signal or noise and more typically more than 10 times background. Even more specifically, an ankyrin repeat domain is said to “specifically bind” a target when the equilibrium dissociation constant (K_{D}) value is <1 μM, such as <100 nM, <10 nM, <100 pM, <10 pM, or <1 pM.

The K_{D} value is often referred to as binding affinity. Binding affinity measures the strength of the sum total of non-covalent interactions between a contact residue of one binding partner (e.g., FAP or 4-1BB binding domains disclosed herein) and a contact residue of its binding partner (e.g., FAP or 4-1BB). Unless indicated otherwise, as used herein, binding affinity refers to binding affinity that reflects a 1:1 interaction between members of a binding pair or binding partners. In case of a binding protein comprising two binding domains for one binding partner, binding affinity may refer to binding affinity that reflects a 1:2 interaction between the binding protein and the binding partner.

A variety of methods of measuring binding affinity are known in the art, any of which can be used for purposes of the present invention. For example, as exemplified herein, the binding affinity can be expressed as K_{D} value, which refers to the dissociation rate of a particular ankyrin repeat domain and its binding target. K_{D} is the ratio of the rate of dissociation, also called the “off-rate (K_{off})”, to the association rate, or “on-rate (Kₒₙ)”. Thus, K_{D} equals K_{off}/Kₒₙ and is expressed as a molar concentration (M), and the smaller the K_{D}, the stronger the affinity of binding.

K_{D} values can be determined using any suitable method. One exemplary method for measuring K_{D} is surface plasmon resonance (SPR) (see, e.g., Nguyen et al. Sensors (Basel). 2015 May 5; 15(5):10481-510). K_{D} value may be measured by SPR using a biosensor system such as a BIACORE® system. BIAcore kinetic analysis comprises analyzing the binding and dissociation of an antigen from chips with immobilized molecules (e.g., molecules comprising epitope binding domains), on their surface. Another method for determining the K_{D} of a protein is by using Bio-Layer Interferometry (see, e.g., Shah et al. J Vis Exp. 2014; (84): 51383). K_{D} value may be measured using OCTET® technology (Octet QKe system, ForteBio). Alternatively or in addition, a KinExA® (Kinetic Exclusion Assay) assay, available from Sapidyne Instruments (Boise, Id.) can also be used. Any method suitable for assessing the binding affinity between two binding partners is encompassed herein.

The term “treat,” as well as words related thereto, does not necessarily imply 100% or complete cure. Rather, there are varying degrees of treatment of which one of ordinary skill in the art recognizes as having a potential benefit or therapeutic effect. In this respect, the methods of treating cancer of the present disclosure can provide any amount or any level of treatment. Furthermore, the treatment provided by the method of the present disclosure can include treatment of (i.e., relief from) one or more conditions or symptoms. Also, the treatment provided by the methods of the present disclosure can encompass slowing the progression of the cancer. For example, the methods can treat cancer by virtue of enhancing the T cell activity or an immune response against the cancer, reducing tumor or cancer growth or appearance of new lesions, reducing metastasis of tumor cells, increasing cell death of tumor or cancer cells, inhibition of tumor or cancer cell survival, and the like. In exemplary aspects, the methods treat by way of delaying the onset or recurrence of the cancer by 1 day, 2 days, 4 days, 6 days, 8 days, 10 days, 15 days, 30 days, two months, 4 months, 6 months, 1 year, 2 years, 4 years, or more. In exemplary aspects, the methods treat by way increasing the survival of the subject. The term “treatment” also includes prophylactic treatment.

Therapeutic responses in any given disease or condition can be determined by standardized response criteria specific to that disease or condition. Tumor response can be assessed using screening techniques such as magnetic resonance imaging (MRI) scan, x-radiographic imaging, computed tomographic (CT) scan, positron emission tomography (PET) scan, bone scan, ultrasound, tumor biopsy sampling, counting of tumor cells in circulation, and/or measurement of tumor antigen (e.g., prostate specific antigen (PSA) and/or alphafeltoprotein (AFP)). In addition to these therapeutic responses, the subject undergoing therapy may experience the beneficial effect of an improvement in the symptoms associated with the disease.

### 3. Multispecific Molecules that Target Fap and 4-1BB

Disclosed herein are multispecific molecules that target FAP and 4-1BB. The molecules are useful for, e.g., treating cancer. The molecules can comprise recombinant proteins.

3.1. Ankyrin Repeat Domains and Ankyrin Repeat Proteins

The ankyrin repeat domains described herein generally comprise at least one ankyrin repeat motif. The ankyrin repeat motif comprises of two anti-parallel α-helices followed by a beta-bulge and beta-hairpin containing loop connecting it to the next repeat, each of which has about 33 residues.

In natural ankyrin repeat proteins, the repeats occur in tandem from several up to 24 repeats (see, e.g., Sedgwick and Smerdon TIBS (1999) 24 311-316). The extended beta-hairpin containing loops, or “fingers”, form a groove on the surface. Over 3500 sequences containing ankyrin motifs can be found listed in the SMART domain database (Shultz et al. PNAS (1998) 95 5857-5864).

Recombinant proteins, or binding domains thereof, comprising designed ankyrin repeat motifs are also referred herein as DARPin® proteins. See Stumpp et al., Curr Opin Drug Discov Devel. 10(2): 153-9 (2007); and Binz et al., Nature Biotech. 22(5): 575-582 (2004). DARPin® proteins can be considered as antibody mimetics with high specificity and high binding affinity to a target protein. In general, a DARPin® protein comprises at least one ankyrin repeat motif, for example, at least 2, 3, or more ankyrin repeat motifs.

The ankyrin repeat domains described herein generally comprise a core scaffold that provides structure, and target binding residues that bind to a target. The structural core includes conserved amino acid residues, and the target binding surface includes amino acid residues that differ depending on the target. For example, an ankyrin repeat motif can comprise the following sequence: DxxGxTPLHLAxxxGxxx|VxVLLxxGADVNAx (SEQ ID NO: 11), wherein “x” denotes any amino acid.

International Patent Publication No. WO 2002/020565 describes a library of ankyrin repeat proteins that can be used for the selection/screening of a protein that binds specifically to a target. Methods of making such libraries are also provided.

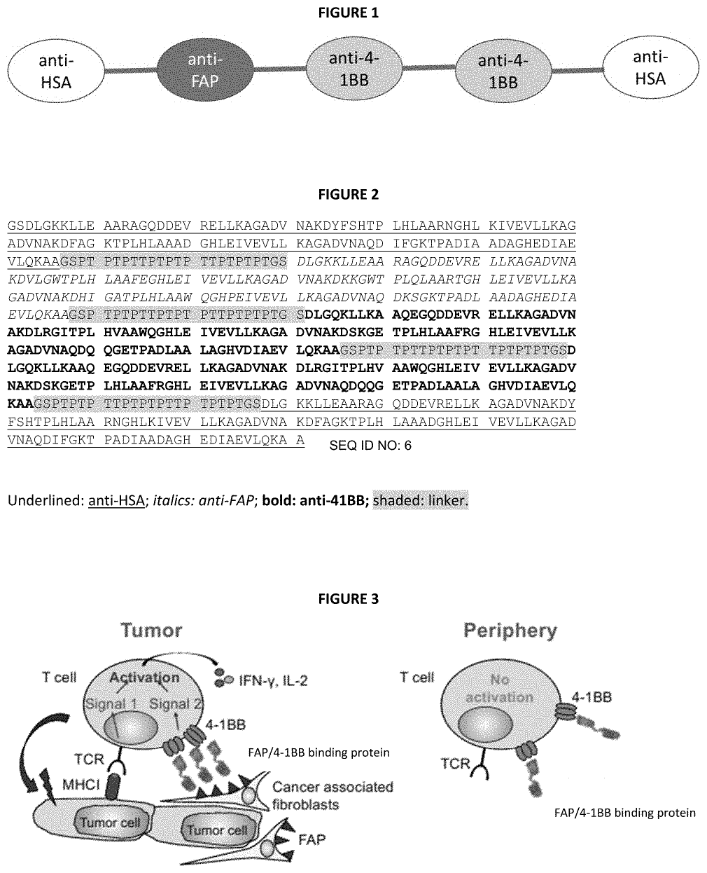

Multiple ankyrin repeat domains can be linked (either through a covalent bond or non-covalent association) to form bispecific or multi-specific molecules. One such molecule is shown in FIG. 1, where one FAP-binding domain and two 4-1BB binding domain are linked to form a multispecific molecule. The molecule also includes two half-life extending moieties, one at N-terminus and one at C-terminus.

3.2. FAP-Binding Domain

One attractive stromal cell target is the fibroblast activation protein (FAP), a transmembrane serine protease highly expressed in the cancer-associated stromal cells of virtually all epithelial cancers. FAP is also expressed during embryonic development, in tissues of healing wounds, and in chronic inflammatory and fibrotic conditions such as liver cirrhosis and idiopathic pulmonary fibrosis. However, FAP has not been detected by immunohistochemistry in benign tumors nor in most normal quiescent adult stromal cells.

The recombinant protein described herein comprises an ankyrin repeat domain that specifically binds FAP, also referred herein as “FAP binding domain”.

In some embodiments, the FAP binding domain described herein comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 2. In an exemplary embodiment, the FAP binding domain described herein comprises an amino acid sequence that is at least 90% identical to SEQ ID NO: 2. In some embodiments, the FAP binding domain described herein comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to any one of SEQ ID NOs: 18-23 and 39-43. In an exemplary embodiment, the FAP binding domain described herein comprises an amino acid sequence that is at least 90% identical to any one of SEQ ID NOs: 18-23 and 39-43.

In some embodiments, no more than 10, no more than 9, no more than 8, no more than 7, no more than 6, no more than 5, no more than 4, no more than 3, no more than 2, or no more than 1 substitution is made relative to the sequence of SEQ ID NO: 2. In some embodiments, no more than 5 substitutions are made relative to the sequence of SEQ ID NO: 2. In some embodiments, no more than 4 substitutions are made relative to the sequence of SEQ ID NO: 2. In some embodiments, no more than 3 substitutions are made relative to the sequence of SEQ ID NO: 2. In some embodiments, no more than 2 substitutions are made relative to the sequence of SEQ ID NO: 2. In some embodiments, no more than 1 substitution is made relative to the sequence of SEQ ID NO: 2. In some embodiments, the substitution(s) do not change the K_{D} value by more than 1000-fold, more than 100-fold, or more than 10-fold, compared to the K_{D} value of the protein comprising the sequence of SEQ ID NO: 2. In certain embodiments, the substitution is a conservative substitution according to Table 1. In certain embodiments, the substitution is made outside the structural core residues of the ankyrin repeat domain, e.g. in the beta loops that connect the alpha-helices. In certain embodiments, the substitution is made within the structural core residues of the ankyrin repeat domain. For example, the ankyrin domain may comprise the consensus sequence: DxxGxTPLHLAxxxGxxx|VxVLLxxGADVNAx (SEQ ID NO: 11), wherein “x” denotes any amino acid (preferably not cysteine, glycine, or proline); or DxxGxTPLHLAAxxGHLEIVEVLLKzGADVNAx (SEQ ID NO: 12), wherein “x” denotes any amino acid (preferably not cysteine, glycine, or proline), and “z” is selected from the group consisting of asparagine, histidine, or tyrosine. In one embodiment, the substitution is made to residues designated as “x”. In another embodiment, the substitution is made outside the residues designated as “x”.

In addition, the second last position can be “A” (see, e.g., SEQ ID NOs: 2, 18-22, and 43) or “L” (see, e.g., SEQ ID NOs: 23 and 39-42), and/or the last position can be “A” (see, e.g., SEQ ID NOs: 2, 18-22, and 43) or “N” (see, e.g., SEQ ID NOs: 23 and 39-42), Accordingly, in some embodiments, the FAP-binding domain comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to any one of SEQ ID NOs: 2, 18-22, and 43, and wherein optionally A at the second last position is substituted with L and/or A at the last position is substituted with N. In an exemplary embodiment, the FAP-binding domain comprises an amino acid sequence that is at least 90% identical to any one of SEQ ID NOs: 2, 18-22, and 43, and wherein optionally A at the second last position is substituted with L, and/or A at the last position is substituted with N. In some embodiments, the FAP-binding domain comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to any one of SEQ ID NOs:23 and 39-42, and wherein optionally L at the second last position is substituted with A, and/or N at the last position is substituted with A. In an exemplary embodiment, the FAP-binding domain comprises an amino acid sequence that is at least 90% identical to any one of SEQ ID NOs:23 and 39-42, and wherein optionally L at the second last position is substituted with A, and/or N at the last position is substituted with A. The sequences may optionally comprise at its N-terminus, a G, an S, or a GS (see below).

In addition, the FAP-binding domain may optionally further comprise a “G,” an “5,” or a “GS” sequence at its N-terminus (compare, e.g., SEQ ID NO:2 and SEQ ID NO:34). Accordingly, in some embodiments, the FAP-binding domain (i) comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to any one of SEQ ID NOs: 2, 18-23, and 39-43, and (ii) further comprises at its N-terminus, a G, an S, or a GS. In an exemplary embodiment, the FAP-binding domain comprises an amino acid sequence that is at least 90% identical to any one of SEQ ID NOs: 2, 18-23, and 39-43, and further comprises at its N-terminus, a G, an S, or a GS. In an exemplary embodiment, the FAP-binding domain comprises an amino acid sequence that is at least 95% identical to any one of SEQ ID NOs: 2, 18-23, and 39-43, and further comprises at its N-terminus, a G, an S, or a GS.

In certain embodiments, the affinity between the recombinant protein and its target (FAP) is described in terms of K_{D}. In exemplary embodiments, the K_{D} is about 10⁻¹ M or less, about 10⁻² M or less, about 10⁻³ M or less, about 10⁻⁴ M or less, about 10⁻⁵ M or less, about 10⁻⁶ M or less, about 10⁻⁷ M or less, about 10⁻⁸ M or less, about 10⁻⁹ M or less, about 10⁻¹⁰ M or less, about 10⁻¹¹ M or less, about 10⁻¹² M or less, about 10⁻¹³ M or less, about 10⁻¹⁴ M or less, from about 10⁻⁵ M to about 10⁻¹⁵ M, from about 10⁻⁶ M to about 10⁻¹⁵ M, from about 10⁻⁷ M to about 10⁻¹⁵ M, from about 10⁻⁸ M to about 10⁻¹⁵ M, from about 10⁻⁹ M to about 10⁻¹⁵ M, from about 10⁻¹⁰ M to about 10⁻¹⁵ M, from about 10⁻⁵ M to about 10⁻¹⁴ M, from about 10⁻⁶ M to about 10⁻¹⁴ M, from about 10⁻⁷ M to about 10⁻¹⁴ M, from about 10⁻⁸ M to about 10⁻¹⁴ M, from about 10⁻⁹ M to about 10⁻¹⁴ M, from about 10⁻¹⁰ M to about 10⁻¹⁴ M, from about 10⁻⁵ M to about 10⁻¹³ M, from about 10⁻⁶ M to about 10⁻¹³ M, from about 10⁻⁷ M to about 10⁻¹³ M, from about 10⁻⁸ M to about 10⁻¹³ M, from about 10⁻⁹ M to about 10⁻¹³ M, or from about 10⁻¹⁰ M to about 10⁻¹³ M.

In exemplary embodiments, the recombinant protein binds FAP with an K_{D} value of, or less than: about 50 nM, about 40 nM, about 30 nM, about 20 nM, about 10 nM, about 5 nM, about 2 nM, about 1 nM, about 900 pM, about 800 pM, about 700 pM, about 600 pM, about 500 pM, about 400 pM, about 300 pM, about 250 pM, about 200 pM, about 150 pM, about 100 pM, about 50 pM, about 40 pM, about 30 pM, about 25 pM, about 20 pM, about 15 pM, about 10 pM, about 5 pM, or about 1 pM. In one exemplary embodiment, the recombinant protein binds FAP with a K_{D} value of less than or equal to about 10 nM. In another exemplary embodiment, the recombinant protein binds FAP with a K_{D} value of less than or equal to about 1 nM.

In certain embodiments, the FAP is human FAP (SEQ ID NO: 14).

TABLE 1
Amino Acid Substitutions

| Original | Conservative | Exemplary |
|---|---|---|
| Residue | Substitutions | Substitutions |
| Ala (A) | Val | Val; Leu; Ile |
| Arg (R) | Lys | Lys; Gln; Asn |
| Asn (N) | Gln | Gln; His; Asp, Lys; Arg |
| Asp (D) | Glu | Glu; Asn |
| Cys (C) | Ser | Ser; Ala |
| Gln (Q) | Asn | Asn; Glu |
| Glu (E) | Asp | Asp; Gln |
| Gly (G) | Ala | Ala |
| His (H) | Arg | Asn; Gln; Lys; Arg |
| Ile (I) | Leu | Leu; Val; Met; Ala; Phe; Norleucine |
| Leu (L) | Ile | Norleucine; Ile; Val; Met; Ala; Phe |
| Lys (K) | Arg | Arg; Gln; Asn |
| Met (M) | Leu | Leu; Phe; Ile |
| Phe (F) | Tyr | Leu; Val; Ile; Ala; Tyr |
| Pro (P) | Ala | Ala |
| Ser (S) | Thr | Thr |
| Thr (T) | Ser | Ser |
| Trp (W) | Tyr | Tyr; Phe |
| Tyr (Y) | Phe | Trp; Phe; Thr; Ser |
| Val (V) | Leu | Ile; Leu; Met; Phe; Ala; Norleucine |

3.3. 4-1BB Binding Domains

The recombinant protein disclosed herein also takes advantage of the T-cell stimulatory activities induced by 4-1BB. Previous studies have indicated that some 4-1BB agonist monoclonal antibodies (mAbs) increase costimlatory molecule expression and markedly enhance cytolytic T lymphocyte responses, resulting in anti-tumor efficacy in various models. 4-1BB monotherapy and combination therapy tumor models have established durable anti-tumor protective T cell memory responses (Lynch, 2008, Immunol Rev. 22: 277-286).

The recombinant protein described herein comprises an ankyrin repeat domain that specifically binds 4-1BB, also referred herein as “4-1BB binding domain”. Like 4-1BB agonist antibodies, the 4-1BB binding domain activates the 4-1BB signaling pathway. The recombinant protein described herein may also comprise more than one 4-1BB binding domain, for example, two or three or more 4-1BB binding domains. Thus, the recombinant protein described herein may comprise a first and a second 4-1BB binding domain, or a first, a second and a third 4-1BB binding domain. The embodiments provided below describe such a first 4-1BB binding domain, second 4-1BB binding domain, and/or third 4-1BB binding domain.

In some embodiments, the 4-1BB binding domain or each of said 4-1BB binding domains independently comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 3. In an exemplary embodiment, the 4-1BB binding domain or each of said 4-1BB binding domains independently comprises an amino acid sequence that is at least 90% identical to SEQ ID NO: 3. In some embodiments, the 4-1BB binding domain or each of said 4-1BB binding domains independently comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to any one of SEQ ID NOs: 24-29 and 51-55. In an exemplary embodiment, the 4-1BB binding domain or each of said 4-1BB binding domains independently comprises an amino acid sequence that is at least 90% identical to any one of SEQ ID NOs: 24-29 and 51-55.

The recombinant proteins described herein may comprise a 4-1BB binding domain comprising the amino acid sequence of SEQ ID NO: 3, or one or more substitutions therein. In some embodiments, no more than 10, no more than 9, no more than 8, no more than 7, no more than 6, no more than 5, no more than 4, no more than 3, no more than 2, or no more than 1 substitution is made relative to the sequence of SEQ ID NO: 3. In some embodiments, no more than 5 substitutions are made relative to the sequence of SEQ ID NO: 3. In some embodiments, no more than 4 substitutions are made relative to the sequence of SEQ ID NO: 3. In some embodiments, no more than 3 substitutions are made relative to the sequence of SEQ ID NO: 3. In some embodiments, no more than 2 substitutions are made relative to the sequence of SEQ ID NO: 3. In some embodiments, no more than 1 substitution is made relative to the sequence of SEQ ID NO: 3. In some embodiments, the substitution(s) do not change the K_{D} value by more than 1000-fold, more than 100-fold, or more than 10-fold, compared to the K_{D} value of the protein comprising the sequence of SEQ ID NO: 3. In certain embodiments, the substitution is a conservative substitution according to Table 1. In certain embodiments, the substitution is made outside the structural core residues of the ankyrin repeat domain, e.g. in the beta loops that connect the alpha-helices. In certain embodiments, the substitution is made within the structural core residues of the ankyrin repeat domain. For example, the ankyrin domain may comprise the consensus sequence: DxxGxTPLHLAxxxGxxx|VxVLLxxGADVNAx (SEQ ID NO: 11), wherein “x” denotes any amino acid (preferably not cysteine, glycine, or proline); or DxxGxTPLHLAAxxGHLEIVEVLLKzGADVNAx (SEQ ID NO: 12), wherein “x” denotes any amino acid (preferably not cysteine, glycine, or proline), and “z” is selected from the group consisting of asparagine, histidine, or tyrosine. In one embodiment, the substitution is made to residues designated as “x”. In another embodiment, the substitution is made outside the residues designated as “x”.

In addition, the second last position can be “A” (see, e.g., SEQ ID NOs:3, 24-28, and 54) or “L” (see, e.g., SEQ ID NOs: 29, 51-53, and 55), and/or the last position can be “A” (see, e.g., SEQ ID NOs:3, 24-28, and 54) or “N” (see, e.g., SEQ ID NOs: 29, 51-53, and 55), Accordingly, in some embodiments, the 4-1BB binding domain comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to any one of SEQ ID NOs: 3, 24-28, and 54, and wherein optionally A at the second last position is substituted with L, and/or A at the last position is substituted with N. In an exemplary embodiment, the 4-1BB binding domain comprises an amino acid sequence that is at least 90% identical to any one of SEQ ID NOs: 3, 24-28, and 54, and wherein optionally A at the second last position is substituted with L, and/or A at the last position is substituted with N. In some embodiments, the 4-1BB-binding domain comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to any one of SEQ ID NOs:29, 51-53, and 55, and wherein optionally L at the second last position is substituted with A, and/or N at the last position is substituted with A. In an exemplary embodiment, the 4-1BB binding domain comprises an amino acid sequence that is at least 90% identical to any one of SEQ ID NOs:29, 51-53, and 55, and wherein optionally L at the second last position is substituted with A, and/or N at the last position is substituted with A. The sequences may optionally comprise at its N-terminus, a G, an S, or a GS (see below).

In addition, the 4-1BB-binding domain may optionally further comprise a “G,” an “S,” or a “GS” sequence at its N-terminus (compare, e.g., SEQ ID NO:3 and SEQ ID NO:35). Accordingly, in some embodiments, the 4-1BB-binding domain comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to any one of SEQ ID NOs: 3, 24-29, and 51-55, and further comprises at its N-terminus, a G, an S, or a GS. In an exemplary embodiment, the 4-1BB-binding domain comprises an amino acid sequence that is at least 90% identical to any one of SEQ ID NOs: 3, 24-29, and 51-55, and further comprises at its N-terminus, a G, an S, or a GS.

In certain embodiments, the affinity between the recombinant protein and its target (4-1BB) is described in terms of K_{D}. In exemplary embodiments, the K_{D} is about 10⁻¹ M or less, about 10⁻² M or less, about 10⁻³ M or less, about 10⁻⁴ M or less, about 10⁻⁵ M or less, about 10⁻⁶ M or less, about 10⁻⁷ M or less, about 10⁻⁸ M or less, about 10⁻⁹ M or less, about 10⁻¹⁰ M or less, about 10⁻¹¹ M or less, about 10⁻¹² M or less, about 10⁻¹³ M or less, about 10⁻¹⁴ M or less, from about 10⁻⁵ M to about 10⁻¹⁵ M, from about 10⁻⁶ M to about 10⁻¹⁵ M, from about 10⁻⁷ M to about 10⁻¹⁵ M, from about 10⁻⁸ M to about 10⁻¹⁵ M, from about 10⁻⁹ M to about 10⁻¹⁵ M, from about 10⁻¹⁰ M to about 10⁻¹⁵ M, from about 10⁻⁵ M to about 10⁻¹⁴ M, from about 10⁻⁶ M to about 10⁻¹⁴ M, from about 10⁻⁷ M to about 10⁻¹⁴ M, from about 10⁻⁸ M to about 10⁻¹⁴ M, from about 10⁻⁹ M to about 10⁻¹⁴ M, from about 10⁻¹⁰ M to about 10⁻¹⁴ M, from about 10⁻⁵ M to about 10⁻¹³ M, from about 10⁻⁶ M to about 10⁻¹³ M, from about 10⁻⁷ M to about 10⁻¹³ M, from about 10⁻⁸ M to about 10⁻¹³ M, from about 10⁻⁹ M to about 10⁻¹³ M, or from about 10⁻¹⁰ M to about 10⁻¹³ M.

In exemplary embodiments, the recombinant protein binds 4-1BB with an K_{D} value of, or less than: about 50 nM, about 40 nM, about 30 nM, about 20 nM, about 10 nM, about 5 nM, about 2 nM, about 1 nM, about 900 pM, about 800 pM, about 700 pM, about 600 pM, about 500 pM, about 400 pM, about 300 pM, about 250 pM, about 200 pM, about 150 pM, about 100 pM, about 50 pM, about 40 pM, about 30 pM, about 25 pM, about 20 pM, about 15 pM, about 10 pM, about 5 pM, or about 1 pM. In some exemplary embodiments, the recombinant protein binds 4-1BB with a K_{D} value of less than or equal to 10 nM. In some exemplary embodiments, the recombinant protein binds 4-1BB with a K_{D} value of less than or equal to 1 nM. In some exemplary embodiments, the recombinant protein binds 4-1BB with a K_{D} value of less than or equal to 50 pM.

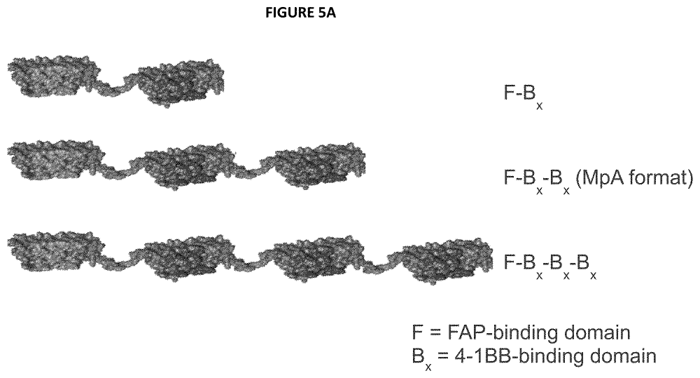

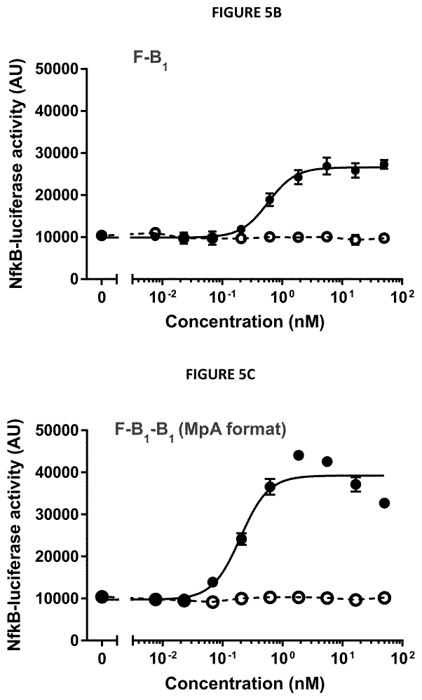

In some embodiments, two or more 4-1BB binding domains are preferred, to further promote 4-1BB clustering and T-cell co-stimulation. It has been reported that 4-1BB ligand binding to 4-1BB on T cells results in trimerization of 4-1BB monomers. However, trimerization alone is not sufficient to activate the 4-1BB receptor. Higher order of clustering is required. As described herein, through FAP-binding, the multispecific molecule already promotes 4-1BB clustering in tumor environment. To further promote 4-1BB clustering, two or more 4-1BB binding domains can be used, to create a “cross-linking” effect on the cell surface. For example, as shown in FIGS. 5A-5B, monovalent 4-1BB binder (F-B) was sufficient to activate the 4-1BB pathway. Higher potency can be achieved by using two 4-1BB binding domains (F-B-B), or three 4-1BB binding domains (F-B-B-B). FIGS. 5A-5B also show that two 4-1BB binding domains are sufficient to activate 4-1BB pathway with high potency, and it is not necessary to have three 4-1BB binding domains for efficient 4-1BB clustering.

In certain embodiments, the 4-1BB is human 4-1BB (SEQ ID NO: 13).

3.4. Half-Life Extending Moieties

The “half-life extending moiety” extends the serum half-life in vivo of the recombinant proteins described herein, compared to the same protein without the half-life extending moiety. Examples of half-life extending moieties include, but are not limited to, polyhistidine, Glu-Glu, glutathione S transferase (GST), thioredoxin, protein A, protein G, an immunoglobulin domain, maltose binding protein (MBP), human serum albumin (HSA) binding domain, or polyethylene glycol (PEG).

In some embodiments, the recombinant multispecific protein described herein comprises an ankyrin repeat domain that specifically binds serum albumin, also referred herein as “serum albumin binding domain”. The recombinant protein described herein may also comprise more than one serum albumin binding domain, for example, two or three or more serum albumin binding domains. Thus, the recombinant protein described herein may comprise a first and a second serum albumin binding domain, or a first, a second and a third serum albumin binding domain. The embodiments provided below describe such a first serum albumin binding domain, second serum albumin binding domain, and/or third serum albumin binding domain.

In some embodiments, the half-life extending moiety described herein comprises a serum albumin binding domain comprising an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 1 or SEQ ID NO:5. In an exemplary embodiment, the half-life extending moiety described herein comprises an amino acid sequence that is at least 90% identical to SEQ ID NO: 1 or SEQ ID NO:5. In some embodiments, the half-life extending moiety described herein comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 30 or SEQ ID NO: 31. In an exemplary embodiment, the half-life extending moiety described herein comprises an amino acid sequence that is at least 90% identical to SEQ ID NO: 30 or SEQ ID NO:31.

Recombinant proteins described herein may comprise a half-life extending moiety comprising the amino acid sequence of SEQ ID NO: 1 or SEQ ID NO: 5, or one or more substitutions therein. In some embodiments, no more than 10, no more than 9, no more than 8, no more than 7, no more than 6, no more than 5, no more than 4, no more than 3, no more than 2, or no more than 1 substitution is made relative to the sequence of SEQ ID NO: 1 or SEQ ID NO:5. In some embodiments, no more than 5 substitutions are made relative to the sequence of SEQ ID NO: 1 or SEQ ID NO:5. In some embodiments, no more than 4 substitutions are made relative to the sequence of SEQ ID NO: 1 or SEQ ID NO:5. In some embodiments, no more than 3 substitutions are made relative to the sequence of SEQ ID NO: 1 or SEQ ID NO:5. In some embodiments, no more than 2 substitutions are made relative to the sequence of SEQ ID NO: 1 or SEQ ID NO:5. In some embodiments, no more than 1 substitution is made relative to the sequence of SEQ ID NO: 1 or SEQ ID NO:5. In some embodiments, the substitution(s) do not change the K_{D} value by more than 1000-fold, more than 100-fold, or more than 10-fold, compared to the K_{D} value of the protein comprising the sequence of SEQ ID NO: 1 or SEQ ID NO:5. In certain embodiments, the substitution is a conservative substitution according to Table 1. In certain embodiments, the substitution is made outside the structural core residues of the ankyrin repeat domain, e.g. in the beta loops that connect the alpha-helices. In certain embodiments, the substitution is made within the structural core residues of the ankyrin repeat domain. For example, the ankyrin domain may comprise the consensus sequence: DxxGxTPLHLAxxxGxxx|VxVLLxxGADVNAx (SEQ ID NO: 11), wherein “x” denotes any amino acid (preferably not cysteine, glycine, or proline); or DxxGxTPLHLAAxxGHLEIVEVLLKzGADVNAx (SEQ ID NO: 12), wherein “x” denotes any amino acid (preferably not cysteine, glycine, or proline), and “z” is selected from the group consisting of asparagine, histidine, or tyrosine. In one embodiment, the substitution is made to residues designated as “x”. In another embodiment, the substitution is made outside the residues designated as “x”.

In addition, the second last position can be “A” or “L”, and/or the last position can be “A” (see, e.g., SEQ ID NOs:1, 5, 30 and 31) or “N” (see, e.g., SEQ ID NO: 36), Accordingly, in some embodiments, the serum albumin binding domain comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to any one of SEQ ID NOs: 1, 5, and 30-31, and wherein optionally A at the second last position is substituted with L and/or A at the last position is substituted with N. In an exemplary embodiment, the serum albumin binding domain comprises an amino acid sequence that is at least 90% identical to any one of SEQ ID NOs: 1, 5, 30, and 31, and wherein optionally A at the second last position is substituted with L and/or A at the last position is substituted with N. The sequences may optionally comprise at its N-terminus, a G, an S, or a GS (see below).

In addition, the serum albumin binding domain may optionally further comprise a “G,” an “5,” or a “GS” sequence at its N-terminus (compare, e.g., SEQ ID NO:1 and SEQ ID NO:5). Accordingly, in some embodiments, the serum albumin binding domain comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to any one of SEQ ID NOs: 1, 30 and 31, and further comprising at its N-terminus, a G, an S, or a GS. In an exemplary embodiment, the serum albumin binding domain comprises an amino acid sequence that is at least 90% identical to any one of SEQ ID NOs: 1, 30, and 31, and further comprises at its N-terminus, a G, an S, or a GS. Furthermore, in some embodiments, the serum albumin binding domain comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 36, and further comprises at its N-terminus, a G, an S, or a GS.

In certain embodiments, the affinity between the recombinant protein and its target (serum albumin) is described in terms of K_{D}. In exemplary embodiments, the K_{D} is about 10⁻¹ M or less, about 10⁻² M or less, about 10⁻³ M or less, about 10⁻⁴ M or less, about 10⁻⁵ M or less, about 10⁻⁶ M or less, about 10⁻⁷ M or less, about 10⁻⁸ M or less, about 10⁻⁹ M or less, about 10⁻¹⁰ M or less, about 10⁻¹¹ M or less, about 10⁻¹² M or less, about 10⁻¹³ M or less, about 10⁻¹⁴ M or less, from about 10⁻⁵ M to about 10⁻¹⁵ M, from about 10⁻⁶ M to about 10⁻¹⁵ M, from about 10⁻⁷ M to about 10⁻¹⁵ M, from about 10⁻⁸ M to about 10⁻¹⁵ M, from about 10⁻⁹ M to about 10⁻¹⁵ M, from about 10⁻¹⁰ M to about 10⁻¹⁵ M, from about 10⁻⁵ M to about 10⁻¹⁴ M, from about 10⁻⁶ M to about 10⁻¹⁴ M, from about 10⁻⁷ M to about 10⁻¹⁴ M, from about 10⁻⁸ M to about 10⁻¹⁴ M, from about 10⁻⁹ M to about 10⁻¹⁴ M, from about 10⁻¹⁰ M to about 10⁻¹⁴ M, from about 10⁻⁵ M to about 10⁻¹³ M, from about 10⁻⁶ M to about 10⁻¹³ M, from about 10⁻⁷ M to about 10⁻¹³ M, from about 10⁻⁸ M to about 10⁻¹³ M, from about 10⁻⁹ M to about 10⁻¹³ M, or from about 10⁻¹⁰ M to about 10⁻¹³ M.

In exemplary embodiments, the recombinant protein binds serum albumin with an K_{D} value of, or less than: about 900 nM, about 800 nM, about 700 nM, about 600 nM, about 500 nM, about 400 nM, about 300 nM, about 250 nM, about 200 nM, about 150 nM, about 100 nM, about 50 nM, about 40 nM, about 30 nM, about 20 nM, about 10 nM, about 5 nM, about 2 nM, about 1 nM, about 900 pM, about 800 pM, about 700 pM, about 600 pM, about 500 pM, about 400 pM, about 300 pM, about 200 pM, about 100 pM, about 10 pM, or about 1 pM. In one exemplary embodiment, the recombinant protein binds serum albumin with a K_{D} value of less than or equal to 100 nM. In another exemplary embodiment, the recombinant protein binds serum albumin with a K_{D} value of less than or equal to 10 nM.

In certain embodiments, the serum albumin is human serum albumin (HSA) (SEQ ID NO: 15).

In some embodiments, two or more serum albumin binding domains are preferred. In exemplary embodiments, one serum albumin binding domain is located at the N-terminus, and one serum albumin binding domain is located at the C-terminus. In exemplary embodiments, the recombinant protein comprises, from the N-terminus to C-terminus: (i) an ankyrin repeat domain that specifically binds serum albumin; (ii) an ankyrin repeat domain that specifically binds FAP, (iii) an ankyrin repeat domain that specifically binds 4-1BB, (iv) an ankyrin repeat domain that specifically binds 4-1BB; and (v) an ankyrin repeat domain that specifically binds serum albumin. In certain embodiments, the N-terminal serum albumin binding domain (also referred to herein as serum albumin binding domain 1) comprises an amino acid sequence that is at least 90%, at least 91%, at least 92%, at least 93%, at least 94%, at least 95%, at least 96%, at least 97%, at least 98%, at least 99%, or 100% identical to SEQ ID NO: 5. In certain embodiments, the C-terminal serum albumin binding domain (also referred to herein as serum albumin binding domain 2) comprises an amino acid sequence that is at least 90%, at least 91%, at least 92%, at least 93%, at least 94%, at least 95%, at least 96%, at least 97%, at least 98%, at least 99%, or 100% identical to SEQ ID NO: 1.

In some embodiments, the half-life extending moiety comprises an immunoglobulin domain. In some embodiments, the immunoglobulin domain comprises an Fc domain. In some embodiments, the Fc domain is derived from any one of the known heavy chain isotypes: IgG (γ), IgM (μ), IgD (δ), IgE (ε), or IgA (α). In some embodiments, the Fc domain is derived from any one of the known heavy chain isotypes or subtypes: IgG₁ (γ1), IgG₂ (γ2), IgG₃ (γ3), IgG₄ (γ4), IgA₁ (α1), IgA₂ (α2). In some embodiments, the Fc domain is the Fc domain of human IgG₁.

In some embodiments, the Fc domain comprises an uninterrupted native sequence (i.e., wild type sequence) of an Fc domain. In some embodiments, the immunoglobulin Fc domain comprises a variant Fc domain resulting in altered biological activity. For example, at least one point mutation or deletion may be introduced into the Fc domain so as to reduce or eliminate the effector activity (e.g., International Patent Publication No. WO 2005/063815), and/or to increase the homogeneity during the production of the recombinant protein. In some embodiments, the Fc domain is the Fc domain of human IgG₁ and comprises one or more of the following effector-null substitutions: L234A, L235A, and G237A (Eu numbering). In some embodiments, the Fc domain does not comprise the lysine located at the C-terminal position of human IgG1 (i.e., K447 by Eu numbering). The absence of the lysine may increase homogeneity during the production of the recombinant protein. In some embodiments, the Fc domain comprises the lysine located at the C-terminal position (K447, Eu numbering).

3.5. Linkers

The recombinant proteins described herein may comprise a linker. A “linker” is a molecule or group of molecules that binds two separate entities (e.g., FAP-binding domain and 4-1BB binding domain) to one another and can provide spacing and flexibility between the two entities such that they are able to achieve a conformation in which they, e.g., specifically bind their respective targets (e.g., FAP and 4-1BB). Protein linkers are particularly preferred, and they may be expressed as a component of the recombinant protein using standard recombinant DNA techniques well-known in the art. For recombinant proteins described herein comprising two or more linkers (for example formulas comprising two or more “(linker)” components), the linkers may all be the same, or some or all of the linkers may be different from each other.

The ankyrin repeat domains can be linked either covalently, for example, by a disulfide bond, a polypeptide bond or a crosslinking agent; or non-covalently, to produce a heterodimeric protein. The recombinant protein can comprise a linker between a FAP binding domain, a 4-1BB binding domain, and the optional half-life extending moiety.

In some embodiments, the linker is a peptidyl linker. In some embodiments, the peptidyl linker comprises about 1 to 30 amino acid residues. Exemplary linkers include, e.g., a glycine rich peptide; a peptide comprising glycine and serine; a peptide having a sequence [Gly-Gly-Ser]ₙ, wherein n is 1, 2, 3, 4, 5, or 6; or a peptide having a sequence [Gly-Gly-Gly-Gly-Ser]ₙ (SEQ ID NO: 16), wherein n is 1, 2, 3, 4, 5, or 6. A glycine rich peptide linker comprises a peptide linker, wherein at least 25% of the residues are glycine. Glycine rich peptide linkers are well known in the art (e.g., Chichili et al. Protein Sci. 2013 February; 22(2): 153-167).

In some embodiments, the peptidyl linker is a proline-threonine rich peptide linker. In an exemplary embodiment, the linker is the proline-threonine rich peptide linker of SEQ ID NO: 4.

In some embodiments, the linker comprises the amino acid sequence of SEQ ID NO: 4.

3.6. N-Terminal and C-Terminal Capping Sequences

The ankyrin repeat domains of the recombinant protein disclosed herein may comprise N-terminal or C-terminal capping sequences. Capping sequences refers to additional polypeptide sequences fused to the N- or C-terminal end of the ankyrin repeat sequence motif(s), wherein said capping sequences form tight tertiary interactions (i.e. tertiary structure interactions) with the ankyrin repeat sequence motif(s), thereby providing a cap that shields the hydrophobic core of the ankyrin repeat domain at the side from exposing to the solvent.

The N- and/or C-terminal capping sequences may be derived from, a capping unit or other structural unit found in a naturally occurring repeat protein adjacent to a repeat unit. Examples of capping sequences are described in International Patent Publication Nos. WO 2002/020565 and WO 2012/069655, in U.S. Patent Publication No. US20130296221, and by Interlandi et al., J Mol Biol. 2008 Jan. 18; 375(3):837-54. Examples of N-terminal ankyrin capping modules (i.e. N-terminal capping repeats) are SEQ ID NOs:7, 9, 10 and examples of ankyrin C-terminal capping modules (i.e. C-terminal capping repeats) includes SEQ ID NO: 8.

In an exemplary embodiment, the N-terminal capping sequence comprises GSDLGKKLLE AARAGQDDEV RILLKAGADV NA (SEQ ID NO:9) or GSDLGKKLLE AARAGQDDEV RELLKAGADV NA (SEQ ID NO:10), wherein the amino acid residue L at position 24 of SEQ ID NO: 9 or SEQ ID NO: 10 is optionally replaced by V, I or A; up to 9, up to 8, up to 7, up to 6, up to 5, up to 4, up to 3, up to 2 or up to 1 amino acids of SEQ ID NO: 9 or SEQ ID NO:10 in other positions than position 24 are optionally exchanged by any amino acids; and wherein G at position 1 and/or S at position 2 of SEQ ID NO: 9 or SEQ ID NO:10 are optionally missing.

3.7. FAP/4-1BB Dual Targeting Bispecific or Multispecific Molecules

In some embodiments, the recombinant protein described herein comprises, from the N-terminus to C-terminus: (i) a first ankyrin repeat domain that specifically binds FAP, (ii) a second ankyrin repeat domain that specifically binds 4-1BB, and (iii) a third ankyrin repeat domain that specifically binds 4-1BB. The second and third ankyrin repeat domains may have identical sequences, or may have different sequences.

In exemplary embodiments, the recombinant protein comprises, from the N-terminus to C-terminus: (FAP binding domain)-(linker)-(4-1BB binding domain)-(linker)-(4-1BB binding domain). In exemplary embodiments, the recombinant protein comprises, from the N-terminus to C-terminus: (serum albumin binding domain)-(linker)-(FAP binding domain)-(linker)-(4-1BB binding domain)-(linker)-(4-1BB binding domain)-(linker)-(serum albumin binding domain).

In some embodiments, the recombinant protein described herein comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 6.

The recombinant protein described herein may comprise the amino acid sequence of SEQ ID NO: 6, or one or more substitutions therein. In some embodiments, no more than 10, no more than 9, no more than 8, no more than 7, no more than 6, no more than 5, no more than 4, no more than 3, no more than 2, or no more than 1 substitution is made relative to the sequence of SEQ ID NO: 6. In some embodiments, no more than 10 substitutions are made relative to the sequence of SEQ ID NO: 6. In some embodiments, no more than 5 substitutions are made relative to the sequence of SEQ ID NO: 6. In some embodiments, no more than 4 substitutions are made relative to the sequence of SEQ ID NO: 6. In some embodiments, no more than 3 substitutions are made relative to the sequence of SEQ ID NO: 6. In some embodiments, no more than 2 substitutions are made relative to the sequence of SEQ ID NO: 6. In some embodiments, no more than 1 substitution is made relative to the sequence of SEQ ID NO: 6. In some embodiments, the substitution(s) do not change the K_{D} value for FAP-binding or 4-1BB binding by more than 1000-fold, more than 100-fold, or more than 10-fold, compared to the K_{D} value of the protein comprising the sequence of SEQ ID NO: 6. In certain embodiments, the substitution is a conservative substitution according to Table 1.

In one embodiment, said recombinant protein comprises an amino acid sequence that is at least 90%, at least 91%, at least 92%, at least 93%, at least 94%, at least 95%, at least 96%, at least 97%, at least 98%, at least 99%, or 100% identical to SEQ ID NO: 6, and binds human FAP, human 4-1BB, and human serum albumin with a K_{D} value of or below 10 nM. In one embodiment, said recombinant protein comprises an amino acid sequence that is at least 90% identical to SEQ ID NO: 6, and binds human FAP, human 4-1BB, and human serum albumin with a K_{D} value of or below 10 nM. In one embodiment, said recombinant protein comprises an amino acid sequence that is at least 93% identical to SEQ ID NO: 6, and binds human FAP, human 4-1BB, and human serum albumin with a K_{D} value of or below 10 nM. In one embodiment, said recombinant protein comprises an amino acid sequence that is at least 95% identical to SEQ ID NO: 6, and binds human FAP, human 4-1BB, and human serum albumin with a K_{D} value of or below 10 nM. In one embodiment, said recombinant protein comprises an amino acid sequence that is at least 98% identical to SEQ ID NO: 6, and binds human FAP, human 4-1BB, and human serum albumin with a K_{D} value of or below 10 nM. In one embodiment, said recombinant protein comprises the amino acid sequence of SEQ ID NO: 6, and binds human FAP, human 4-1BB, and human serum albumin with a K_{D} value of or below 10 nM. In one embodiment, said recombinant protein comprises the amino acid sequence of SEQ ID NO: 6, and binds human FAP, human 4-1BB, and human serum albumin with a K_{D} value of or below 10 nM, and wherein said recombinant protein has a terminal half-life in a cynomolgus monkey model of at least 1 day, at least 2 days, at least 3 days, at least 4 days, or about 2.8 days, or about 4.5 days, wherein typically and preferably, said terminal half-life in cynomolgus monkey is measured as described in Example 6. In one embodiment, said recombinant protein comprises the amino acid sequence of SEQ ID NO: 6, and binds human FAP, human 4-1BB, and human serum albumin with a K_{D} value of or below 10 nM, and wherein in the presence of said recombinant protein, FAP protease activity is reduced by no more than 25%, no more than 20%, no more than 15%, no more than 10%, no more than 9%, no more than 8%, no more than 7%, no more than 6%, no more than 5%, no more than 4%, no more than 3%, or no more than 2%, as compared to a control, wherein typically and preferably said control is the FAP protease activity in the absence of said recombinant protein, and wherein further typically and preferably said FAP protease activity is measured as described in Example 10. In one embodiment, said recombinant protein comprises the amino acid sequence of SEQ ID NO: 6, and binds human FAP, human 4-1BB, and human serum albumin with a K_{D} value of or below 10 nM, and wherein said recombinant protein has a terminal half-life in a cynomolgus monkey model of at least 1 day, at least 2 days, at least 3 days, at least 4 days, or about 2.8 days, or about 4.5 days, wherein typically and preferably, said terminal half-life in cynomolgus monkey is measured as described in Example 6, and wherein in the presence of said recombinant protein, FAP protease activity is reduced by no more than 25%, no more than 20%, no more than 15%, no more than 10%, no more than 9%, no more than 8%, no more than 7%, no more than 6%, no more than 5%, no more than 4%, no more than 3%, or no more than 2%, as compared to a control, wherein typically and preferably said control is the FAP protease activity in the absence of said recombinant protein, and wherein further typically and preferably said FAP protease activity is measured as described in Example 10.

In certain embodiments, the multispecific recombinant protein induces cytotoxicity upon binding to FAP and 4-1BB. In certain embodiments, the cytotoxicity is T-cell mediated cytotoxicity. In certain embodiments, the T cell is a CD8+ T cell. In certain embodiments, the biological activity of the multispecific recombinant protein is assessed by an in vitro assay that measures cytokine releases. It has been reported that production of cytokines (IFN-γ, TNF-alpha and IL-2) in serum and cytotoxic T lymphocyte (CTL) activity is indicative of 4-1BB activation.

In certain embodiments, the multispecific recombinant protein has a half maximal effective concentration (E050) of no more than about 100 nM, no more than about 75 nM, no more than about 65 nM, no more than about 55 nM, no more than about 45 nM, no more than about 35 nM, no more than about 25 nM, no more than about 15 nM, no more than about 10 nM, no more than about 5 nM, no more than about 4 nM, no more than about 3 nM, no more than about 2 nM, no more than about 1 nM, from about 0.01 nM to about 50 nM, from about 0.01 nM to about 25 nM, from about 0.01 nM to about 10 nM, from about 0.01 nM to about 5 nM, from about 0.05 nM to about 50 nM, from about 0.05 nM to about 25 nM, from about 0.05 nM to about 10 nM, from about 0.05 nM to about 5 nM, from about 0.1 nM to about 50 nM, from about 0.1 nM to about 25 nM, from about 0.1 nM to about 10 nM, from about 0.1 nM to about 5 nM, from about 0.4 nM to about 2 nM, as assessed by an in vitro IFNγ release assay.

In an exemplary embodiment, the multispecific recombinant protein has an EC₅₀ of no more than about 10 nM. In another exemplary embodiment, the multispecific recombinant protein has an EC₅₀ of no more than about 3 nM. In another exemplary embodiments, the multispecific recombinant protein has an EC₅₀ of from about 0.1 nM to about 10 nM.

In certain embodiments, the IFNγ release assay is a human T cell IFNγ release assay. In certain embodiments, the T cell is CD8+ T cell. In an exemplary embodiment, IFNγ release assay is measured using Human IFN-gamma DuoSet ELISA (R&D systems, catalog number DY285B), according to the manufacturer's instructions. In an exemplary embodiment, EC₅₀ value is determined by fitting the data with the four-parameter logistical fit model using Graphpad Prism software. In an exemplary embodiment, EC₅₀ value is determined using the method described in Example 4.

In certain embodiments, the multispecific recombinant protein has a terminal half-life in a mouse model of at least 10 hours, at least 20 hours, at least 30 hours, at least 40 hours, or about 44 hours. In an exemplary embodiment, the terminal half-life in mouse is measured using methods as exemplified in Example 5. In certain embodiments, the multispecific recombinant protein has a terminal half-life in a cynomolgus monkey model of at least 1 day, at least 2 days, at least 3 days, at least 4 days, or about 2.8 days, or about 4.5 days. In an exemplary embodiment, the terminal half-life in cynomolgus monkey is measured using methods as exemplified in Example 6.

In certain embodiments, the multispecific recombinant protein does not inhibit FAP protease activity. In certain embodiments, in the presence of the multispecific recombinant protein, FAP protease activity is reduced by no more than 25%, by no more than 20%, by no more than 15%, by no more than 10%, no more than 9%, no more than 8%, no more than 7%, no more than 6%, no more than 5%, no more than 4%, no more than 3%, or no more than 2%, as compared to a control (the control can be the FAP protease activity in the absence of the multispecific recombinant protein). In an exemplary embodiment, the FAP activity is measured using method as exemplified in Example 10.

In certain embodiments, the recombinant protein described here comprises a FAP binding domain and a 4-1BB binding domain, wherein the 4-1BB binding domain comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to any one of SEQ ID NOs: 3, 24-29, and 51-55; its N-terminus optionally further comprises a G, an S, or a GS; and the second last position can be L or A, and the last position can be N or A. In an exemplary embodiment, the recombinant protein comprises a FAP binding domain and a 4-1BB binding domain, wherein the 4-1BB binding domain comprises an amino acid sequence that is at least 90% identical to any one of SEQ ID NOs: 3, 24-29, and 51-55. The recombinant protein may comprise 1, 2, or 3 such 4-1BB binding domains. The recombinant protein may further comprise a serum albumin binding domain at the N-terminus, the C-terminus, or both.

In certain embodiments, the recombinant protein described here comprises a FAP binding domain and a 4-1BB binding domain, wherein the FAP binding domain comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to any one of SEQ ID NOs: 2, 18-23, and 39-43; its N-terminus optionally further comprises a G, an S, or a GS; and the second last position can be L or A, and the last position can be N or A. In an exemplary embodiment, the recombinant protein comprises a FAP binding domain and a 4-1BB binding domain, wherein the FAP binding domain comprises an amino acid sequence that is at least 90% identical to any one of SEQ ID NOs: 2, 18-23, and 39-43. The recombinant protein may further comprise a serum albumin binding domain at the N-terminus, the C-terminus, or both.

In certain embodiments, the recombinant protein described here comprises a FAP binding domain and a 4-1BB binding domain, wherein: (a) the FAP binding domain comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to any one of SEQ ID NOs: 2, 18-23, and 39-43; wherein its N-terminus optionally further comprises a G, an S, or a GS, and wherein the second last position can be L or A, and/or the last position can be N or A; and (b) said 4-1BB binding domain comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to any one of SEQ ID NOs: 3, 24-29, and 51-55; wherein its N-terminus optionally further comprises a G, an S, or a GS, and wherein the second last position can be L or A, and/or the last position can be N or A. In an exemplary embodiment, the FAP binding domain comprises an amino acid sequence that is at least 90% identical to any one of SEQ ID NOs: 2, 18-23, and 39-43. In an exemplary embodiment, the 4-1BB binding domain comprises an amino acid sequence that is at least 90% identical to any one of SEQ ID NOs: 3, 24-29, and 51-55. In an exemplary embodiment, the recombinant protein described here comprises a FAP binding domain and a 4-1BB binding domain, wherein the FAP binding domain comprises an amino acid sequence that is at least 90% identical to any one of SEQ ID NOs: 2, 18-23, and 39-43, and the 4-1BB binding domain comprises an amino acid sequence that is at least 90% identical to any one of SEQ ID NOs: 3, 24-29, and 51-55. The recombinant protein may comprise 1, 2, or 3 such 4-1BB binding domains. The recombinant protein may further comprise a serum albumin binding domain at the N-terminus, the C-terminus, or both.

In certain embodiments, the recombinant protein described here comprises a FAP binding domain and a 4-1BB binding domain, wherein: (a) the FAP binding domain comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 2; wherein its N-terminus optionally further comprises a G, an S, or a GS; and wherein the second last position can be L or A, and the last position can be N or A; and (b) said 4-1BB binding domain comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 3; wherein its N-terminus optionally further comprises a G, an S, or a GS; and wherein the second last position can be L or A, and the last position can be N or A. In exemplary embodiments, the recombinant protein may further have any one or any combination of the following properties: (i) the FAP binding domain comprises an amino acid sequence that is at least 90% identical to SEQ ID NO: 2; (ii) the 4-1BB binding domain comprises an amino acid sequence that is at least 90% identical to SEQ ID NO: 3; (iii) the FAP binding domain is located at the N-terminus of the 4-1BB binding domain; (iv) the recombinant protein binds human 4-1BB in PBS with a dissociation constant (K_{D}) below 10⁷M, below 10⁻⁸M, below 5×10⁻⁹M, or below 3×10⁻⁹M; (v) the recombinant protein binds human FAP in PBS with a dissociation constant (K_{D}) below 10⁻⁸M, below 5×10⁻⁹M, below 3×10⁻⁹M, or below 1×10⁻⁹M; (vi) the recombinant protein activates human 4-1BB in 4-1BB-expressing HT1080 cells in the presence of FAP-expressing CHO cells with an EC₅₀ value of about 10⁻⁸M or less, or about 10⁻⁹M or less; (vii) said FAP binding domain and said 4-1BB binding domain are linked by a peptidyl linker, preferably a proline-threonine (PT) rich linker (such as a linker comprising SEQ ID NO:4) or a GS linker; (viii) the recombination protein comprises one, two, or three 4-1BB binding domains; (ix) the recombinant protein comprises a serum albumin binding domain at the N-terminus, the C-terminus, or both; (x) the recombinant binding protein is capable of binding FAP, 4-1BB and serum albumin simultaneously, (xi) the recombinant protein does not inhibit FAP protease activity, or the reduction in FAP protease activity in the presence of the recombinant protein is no more than 25%, no more than 20%, no more than 15%, or no more than 10%; (xii) the recombinant protein has a terminal half-life in a mouse model of at least 10 hours, at least 20 hours, at least 30 hours, at least 40 hours, or about 44 hours, and (xiii) the recombinant protein has a terminal half-life in a cynomolgus monkey model of at least 1 day, at least 2 days, at least 3 days, at least 4 days, or about 2.8 days, or about 4.5 days.

In certain embodiments, the recombinant protein described here comprises a FAP binding domain and a 4-1BB binding domain, wherein: (a) the FAP binding domain comprises an amino acid sequence that is at least 90% identical to SEQ ID NO: 2; wherein its N-terminus optionally further comprises a G, an S, or a GS; and wherein the second last position can be L or A, and the last position can be N or A; and (b) said 4-1BB binding domain comprises an amino acid sequence that is at least 90% identical to SEQ ID NO: 3; wherein its N-terminus optionally further comprises a G, an S, or a GS; and wherein the second last position can be L or A, and the last position can be N or A; and (c) the FAP binding domain is located at the N-terminus of the 4-1BB binding domain. The recombinant protein may comprise 1, 2, or 3 such 4-1BB binding domains. The recombinant protein may further comprise a serum albumin binding domain at the N-terminus, the C-terminus, or both.

In certain embodiments, the recombinant protein described here comprises a FAP binding domain and a 4-1BB binding domain, wherein: (a) the FAP binding domain comprises an amino acid sequence that is at least 93% identical to SEQ ID NO: 2; wherein its N-terminus optionally further comprises a G, an S, or a GS; and wherein the second last position can be L or A, and the last position can be N or A; and (b) said 4-1BB binding domain comprises an amino acid sequence that is at least 93% identical to SEQ ID NO: 3; wherein its N-terminus optionally further comprises a G, an S, or a GS; and wherein the second last position can be L or A, and the last position can be N or A; and (c) the FAP binding domain is located at the N-terminus of the 4-1BB binding domain. The recombinant protein may comprise 1, 2, or 3 such 4-1BB binding domains. The recombinant protein may further comprise a serum albumin binding domain at the N-terminus, the C-terminus, or both.

In certain embodiments, the recombinant protein described here comprises a FAP binding domain and a 4-1BB binding domain, wherein: (a) the FAP binding domain comprises an amino acid sequence that is at least 95% identical to SEQ ID NO: 2; wherein its N-terminus optionally further comprises a G, an S, or a GS; and wherein the second last position can be L or A, and the last position can be N or A; and (b) said 4-1BB binding domain comprises an amino acid sequence that is at least 95% identical to SEQ ID NO: 3; wherein its N-terminus optionally further comprises a G, an S, or a GS; and wherein the second last position can be L or A, and the last position can be N or A; and (c) the FAP binding domain is located at the N-terminus of the 4-1BB binding domain. The recombinant protein may comprise 1, 2, or 3 such 4-1BB binding domains. The recombinant protein may further comprise a serum albumin binding domain at the N-terminus, the C-terminus, or both.

In certain embodiments, the recombinant protein described here comprises a FAP binding domain and a 4-1BB binding domain, wherein: (a) the FAP binding domain comprises an amino acid sequence that is at least 98% identical to SEQ ID NO: 2; wherein its N-terminus optionally further comprises a G, an S, or a GS; and wherein the second last position can be L or A, and the last position can be N or A; and (b) said 4-1BB binding domain comprises an amino acid sequence that is at least 98% identical to SEQ ID NO: 3; wherein its N-terminus optionally further comprises a G, an S, or a GS; and wherein the second last position can be L or A, and the last position can be N or A; and (c) the FAP binding domain is located at the N-terminus of the 4-1BB binding domain. The recombinant protein may comprise 1, 2, or 3 such 4-1BB binding domains. The recombinant protein may further comprise a serum albumin binding domain at the N-terminus, the C-terminus, or both.

In certain embodiments, the recombinant protein described here comprises a FAP binding domain and a 4-1BB binding domain, wherein: (a) the FAP binding domain comprises the amino acid sequence of SEQ ID NO: 2; wherein its N-terminus optionally further comprises a G, an S, or a GS; and wherein the second last position can be L or A, and the last position can be N or A; and (b) said 4-1BB binding domain comprises the amino acid sequence of SEQ ID NO: 3; wherein its N-terminus optionally further comprises a G, an S, or a GS; and wherein the second last position can be L or A, and the last position can be N or A; and (c) the FAP binding domain is located at the N-terminus of the 4-1BB binding domain. The recombinant protein may comprise 1, 2, or 3 such 4-1BB binding domains. The recombinant protein may further comprise a serum albumin binding domain at the N-terminus, the C-terminus, or both.

In certain embodiments, the recombinant protein described here comprises a FAP binding domain and a 4-1BB binding domain, wherein: (a) the FAP binding domain comprises an amino acid sequence that is at least 90% identical to SEQ ID NO: 2; wherein its N-terminus optionally further comprises a G, an S, or a GS; and wherein the second last position can be L or A, and the last position can be N or A; and (b) said 4-1BB binding domain comprises an amino acid sequence that is at least 90% identical to SEQ ID NO: 3; wherein its N-terminus optionally further comprises a G, an S, or a GS; and wherein the second last position can be L or A, and the last position can be N or A; and (c) the recombinant protein binds human 4-1BB in PBS with a dissociation constant (K_{D}) value below 10⁻⁷M, below 10⁻⁸M, below 5×10⁻⁹M, or below 3×10⁻⁹M, and/or the recombinant protein binds human FAP in PBS with a dissociation constant (K_{D}) value below 10⁻⁸M, below 5×10⁻⁹M, below 3×10⁻⁹M, or below 1×10⁻⁹M. The recombinant protein may comprise 1, 2, or 3 such 4-1BB binding domains. The recombinant protein may further comprise a serum albumin binding domain at the N-terminus, the C-terminus, or both.

In certain embodiments, the recombinant protein described here comprises a FAP binding domain and a 4-1BB binding domain, wherein: (a) the FAP binding domain comprises an amino acid sequence that is at least 93% identical to SEQ ID NO: 2; wherein its N-terminus optionally further comprises a G, an S, or a GS; and wherein the second last position can be L or A, and the last position can be N or A; and (b) said 4-1BB binding domain comprises an amino acid sequence that is at least 93% identical to SEQ ID NO: 3; wherein its N-terminus optionally further comprises a G, an S, or a GS; and wherein the second last position can be L or A, and the last position can be N or A; and (c) the recombinant protein binds human 4-1BB in PBS with a dissociation constant (K_{D}) value below 10⁻⁷M, below 10⁻⁸M, below 5×10⁻⁹M, or below 3×10⁻⁹M, and/or the recombinant protein binds human FAP in PBS with a dissociation constant (K_{D}) value below 10⁻⁸M, below 5×10⁻⁹M, below 3×10⁻⁹M, or below 1×10⁻⁹M. The recombinant protein may comprise 1, 2, or 3 such 4-1BB binding domains. The recombinant protein may further comprise a serum albumin binding domain at the N-terminus, the C-terminus, or both.

In certain embodiments, the recombinant protein described here comprises a FAP binding domain and a 4-1BB binding domain, wherein: (a) the FAP binding domain comprises an amino acid sequence that is at least 95% identical to SEQ ID NO: 2; wherein its N-terminus optionally further comprises a G, an S, or a GS; and wherein the second last position can be L or A, and the last position can be N or A; and (b) said 4-1BB binding domain comprises an amino acid sequence that is at least 95% identical to SEQ ID NO: 3; wherein its N-terminus optionally further comprises a G, an S, or a GS; and wherein the second last position can be L or A, and the last position can be N or A; and (c) the recombinant protein binds human 4-1BB in PBS with a dissociation constant (K_{D}) value below 10⁻⁷M, below 10⁻⁸M, below 5×10⁻⁹M, or below 3×10⁻⁹M, and/or the recombinant protein binds human FAP in PBS with a dissociation constant (K_{D}) value below 10⁻⁸M, below 5×10⁻⁹M, below 3×10⁻⁹M, or below 1×10⁻⁹M. The recombinant protein may comprise 1, 2, or 3 such 4-1BB binding domains. The recombinant protein may further comprise a serum albumin binding domain at the N-terminus, the C-terminus, or both.

In certain embodiments, the recombinant protein described here comprises a FAP binding domain and a 4-1BB binding domain, wherein: (a) the FAP binding domain comprises an amino acid sequence that is at least 98% identical to SEQ ID NO: 2; wherein its N-terminus optionally further comprises a G, an S, or a GS; and wherein the second last position can be L or A, and the last position can be N or A; and (b) said 4-1BB binding domain comprises an amino acid sequence that is at least 98% identical to SEQ ID NO: 3; wherein its N-terminus optionally further comprises a G, an S, or a GS; and wherein the second last position can be L or A, and the last position can be N or A; and (c) the recombinant protein binds human 4-1BB in PBS with a dissociation constant (K_{D}) value below 10⁻⁷M, below 10⁻⁸M, below 5×10⁻⁹M, or below 3×10⁻⁹M, and/or the recombinant protein binds human FAP in PBS with a dissociation constant (K_{D}) value below 10⁻⁸M, below 5×10⁻⁹M, below 3×10⁻⁹M, or below 1×10⁻⁹M. The recombinant protein may comprise 1, 2, or 3 such 4-1BB binding domains. The recombinant protein may further comprise a serum albumin binding domain at the N-terminus, the C-terminus, or both.

In certain embodiments, the recombinant protein described here comprises a FAP binding domain and a 4-1BB binding domain, wherein: (a) the FAP binding domain comprises the amino acid sequence of SEQ ID NO: 2; wherein its N-terminus optionally further comprises a G, an S, or a GS; and wherein the second last position can be L or A, and the last position can be N or A; and (b) said 4-1BB binding domain comprises the amino acid sequence of SEQ ID NO: 3; wherein its N-terminus optionally further comprises a G, an S, or a GS; and wherein the second last position can be L or A, and the last position can be N or A; and (c) the recombinant protein binds human 4-1BB in PBS with a dissociation constant (K_{D}) value below 10⁻⁷M, below 10⁻⁸M, below 5×10⁻⁹M, or below 3×10⁻⁹M, and/or the recombinant protein binds human FAP in PBS with a dissociation constant (K_{D}) value below 10⁻⁸M, below 5×10⁻⁹M, below 3×10⁻⁹M, or below 1×10⁻⁹M. The recombinant protein may comprise 1, 2, or 3 such 4-1BB binding domains. The recombinant protein may further comprise a serum albumin binding domain at the N-terminus, the C-terminus, or both.

In certain embodiments, the recombinant protein described here comprises a FAP binding domain and a 4-1BB binding domain, wherein: (a) the FAP binding domain comprises an amino acid sequence that is at least 90% identical to SEQ ID NO: 2; wherein its N-terminus optionally further comprises a G, an S, or a GS; and wherein the second last position can be L or A, and the last position can be N or A; and (b) said 4-1BB binding domain comprises an amino acid sequence that is at least 90% identical to SEQ ID NO: 3; wherein its N-terminus optionally further comprises a G, an S, or a GS; and wherein the second last position can be L or A, and the last position can be N or A; and (c) the recombinant protein binds human 4-1BB in PBS with a dissociation constant (K_{D}) value below 5×10⁻⁹M, and/or the recombinant protein binds human FAP in PBS with a dissociation constant (K_{D}) value below 5×10⁻⁹M. The recombinant protein may comprise 1, 2, or 3 such 4-1BB binding domains. The recombinant protein may further comprise a serum albumin binding domain at the N-terminus, the C-terminus, or both.

In certain embodiments, the recombinant protein described here comprises a FAP binding domain and a 4-1BB binding domain, wherein: (a) the FAP binding domain comprises an amino acid sequence that is at least 95% identical to SEQ ID NO: 2; wherein its N-terminus optionally further comprises a G, an S, or a GS; and wherein the second last position can be L or A, and the last position can be N or A; and (b) said 4-1BB binding domain comprises an amino acid sequence that is at least 95% identical to SEQ ID NO: 3; wherein its N-terminus optionally further comprises a G, an S, or a GS; and wherein the second last position can be L or A, and the last position can be N or A; and (c) the recombinant protein binds human 4-1BB in PBS with a dissociation constant (K_{D}) value below 5×10⁻⁹M, and/or the recombinant protein binds human FAP in PBS with a dissociation constant (K_{D}) value below 5×10⁻⁹M. The recombinant protein may comprise 1, 2, or 3 such 4-1BB binding domains. The recombinant protein may further comprise a serum albumin binding domain at the N-terminus, the C-terminus, or both.

In certain embodiments, the recombinant protein described here comprises a FAP binding domain and a 4-1BB binding domain, wherein: (a) the FAP binding domain comprises the amino acid sequence of SEQ ID NO: 2; wherein its N-terminus optionally further comprises a G, an S, or a GS; and wherein the second last position can be L or A, and the last position can be N or A; and (b) said 4-1BB binding domain comprises the amino acid sequence of SEQ ID NO: 3; wherein its N-terminus optionally further comprises a G, an S, or a GS; and wherein the second last position can be L or A, and the last position can be N or A; and (c) the recombinant protein binds human 4-1BB in PBS with a dissociation constant (K_{D}) value below 5×10⁻⁹M, and/or the recombinant protein binds human FAP in PBS with a dissociation constant (K_{D}) value below 5×10⁻⁹M. The recombinant protein may comprise 1, 2, or 3 such 4-1BB binding domains. The recombinant protein may further comprise a serum albumin binding domain at the N-terminus, the C-terminus, or both.

In certain embodiments, the recombinant protein described here comprises a FAP binding domain and a 4-1BB binding domain, wherein: (a) the FAP binding domain comprises an amino acid sequence that is at least 90% identical to SEQ ID NO: 2; wherein its N-terminus optionally further comprises a G, an S, or a GS; and wherein the second last position can be L or A, and the last position can be N or A; and (b) said 4-1BB binding domain comprises an amino acid sequence that is at least 90% identical to SEQ ID NO: 3; wherein its N-terminus optionally further comprises a G, an S, or a GS; and wherein the second last position can be L or A, and the last position can be N or A; and (c) the recombinant protein activates human 4-1BB in 4-1BB-expressing HT1080 cells in the presence of FAP-expressing CHO cells with an EC₅₀ value of about 10⁻⁸M or less, or about 10⁻⁹M or less. The recombinant protein may comprise 1, 2, or 3 such 4-1BB binding domains. The recombinant protein may further comprise a serum albumin binding domain at the N-terminus, the C-terminus, or both.

In certain embodiments, the recombinant protein described here comprises a FAP binding domain and a 4-1BB binding domain, wherein: (a) the FAP binding domain comprises an amino acid sequence that is at least 93% identical to SEQ ID NO: 2; wherein its N-terminus optionally further comprises a G, an S, or a GS; and wherein the second last position can be L or A, and the last position can be N or A; and (b) said 4-1BB binding domain comprises an amino acid sequence that is at least 93% identical to SEQ ID NO: 3; wherein its N-terminus optionally further comprises a G, an S, or a GS; and wherein the second last position can be L or A, and the last position can be N or A; and (c) the recombinant protein activates human 4-1BB in 4-1BB-expressing HT1080 cells in the presence of FAP-expressing CHO cells with an EC₅₀ value of about 10⁻⁸M or less, or about 10⁻⁹M or less. The recombinant protein may comprise 1, 2, or 3 such 4-1BB binding domains. The recombinant protein may further comprise a serum albumin binding domain at the N-terminus, the C-terminus, or both.

In certain embodiments, the recombinant protein described here comprises a FAP binding domain and a 4-1BB binding domain, wherein: (a) the FAP binding domain comprises an amino acid sequence that is at least 95% identical to SEQ ID NO: 2; wherein its N-terminus optionally further comprises a G, an S, or a GS; and wherein the second last position can be L or A, and the last position can be N or A; and (b) said 4-1BB binding domain comprises an amino acid sequence that is at least 95% identical to SEQ ID NO: 3; wherein its N-terminus optionally further comprises a G, an S, or a GS; and wherein the second last position can be L or A, and the last position can be N or A; and (c) the recombinant protein activates human 4-1BB in 4-1BB-expressing HT1080 cells in the presence of FAP-expressing CHO cells with an EC₅₀ value of about 10⁻⁸M or less, or about 10⁻⁹M or less. The recombinant protein may comprise 1, 2, or 3 such 4-1BB binding domains. The recombinant protein may further comprise a serum albumin binding domain at the N-terminus, the C-terminus, or both.

In certain embodiments, the recombinant protein described here comprises a FAP binding domain and a 4-1BB binding domain, wherein: (a) the FAP binding domain comprises an amino acid sequence that is at least 98% identical to SEQ ID NO: 2; wherein its N-terminus optionally further comprises a G, an S, or a GS; and wherein the second last position can be L or A, and the last position can be N or A; and (b) said 4-1BB binding domain comprises an amino acid sequence that is at least 98% identical to SEQ ID NO: 3; wherein its N-terminus optionally further comprises a G, an S, or a GS; and wherein the second last position can be L or A, and the last position can be N or A; and (c) the recombinant protein activates human 4-1BB in 4-1BB-expressing HT1080 cells in the presence of FAP-expressing CHO cells with an EC₅₀ value of about 10⁻⁸M or less, or about 10⁻⁹M or less. The recombinant protein may comprise 1, 2, or 3 such 4-1BB binding domains. The recombinant protein may further comprise a serum albumin binding domain at the N-terminus, the C-terminus, or both.

In certain embodiments, the recombinant protein described here comprises a FAP binding domain and a 4-1BB binding domain, wherein: (a) the FAP binding domain comprises the amino acid sequence of SEQ ID NO: 2; wherein its N-terminus optionally further comprises a G, an S, or a GS; and wherein the second last position can be L or A, and the last position can be N or A; and (b) said 4-1BB binding domain comprises the amino acid sequence of SEQ ID NO: 3; wherein its N-terminus optionally further comprises a G, an S, or a GS; and wherein the second last position can be L or A, and the last position can be N or A; and (c) the recombinant protein activates human 4-1BB in 4-1BB-expressing HT1080 cells in the presence of FAP-expressing CHO cells with an EC₅₀ value of about 10⁻⁸M or less, or about 10⁻⁹M or less. The recombinant protein may comprise 1, 2, or 3 such 4-1BB binding domains. The recombinant protein may further comprise a serum albumin binding domain at the N-terminus, the C-terminus, or both.

In certain embodiments, the recombinant protein described here comprises a FAP binding domain and a 4-1BB binding domain, wherein: (a) the FAP binding domain comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 2; wherein its N-terminus optionally further comprises a G, an S, or a GS; and wherein the second last position can be L or A, and the last position can be N or A; and (b) said 4-1BB binding domain comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 3; wherein its N-terminus optionally further comprises a G, an S, or a GS; and wherein the second last position can be L or A, and the last position can be N or A; and (c) said FAP binding domain and said 4-1BB binding domain are linked by a peptidyl linker, preferably a proline-threonine (PT) rich linker (such as a linker comprising SEQ ID NO:4) or a GS linker. The recombinant protein may comprise 1, 2, or 3 such 4-1BB binding domains. The recombinant protein may further comprise a serum albumin binding domain at the N-terminus, the C-terminus, or both.

In certain embodiments, the recombinant protein described here comprises a FAP binding domain and two 4-1BB binding domains, wherein: (a) the FAP binding domain comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 2; wherein its N-terminus optionally further comprises a G, an S, or a GS; and wherein the second last position can be L or A, and the last position can be N or A; and (b) each of said two 4-1BB binding domains independently comprises an amino acid sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to SEQ ID NO: 3; wherein its N-terminus optionally further comprises a G, an S, or a GS; and wherein the second last position can be L or A, and the last position can be N or A. In exemplary embodiments, the recombinant protein may further have any one or any combination of the following properties: (i) the FAP binding domain comprises an amino acid sequence that is at least 90% identical to SEQ ID NO: 2; (ii) each of the 4-1BB binding domains independently comprises an amino acid sequence that is at least 90% identical to SEQ ID NO: 3; (iii) the recombinant protein comprises, from the N-terminus to C-terminus: (FAP binding domain)-(4-1BB binding domain)-(4-1BB binding domain); (iv) recombinant protein binds human 4-1BB in PBS with a dissociation constant (K_{D}) value below 10⁻⁷M, below 10⁻⁸M, below 5×10⁻⁹M, or below 3×10⁻⁹M; (v) the recombinant protein binds human FAP in PBS with a dissociation constant (K_{D}) below 10⁻⁸M, below 5×10⁻⁹M, below 3×10⁻⁹M, or below 1×10⁻⁹M; (vi) the recombinant protein activates human 4-1BB in 4-1BB-expressing HT1080 cells in the presence of FAP-expressing CHO cells with an EC₅₀ value of about 10⁻⁸M or less, or about 10⁻⁹M or less; (vii) said FAP binding domain and said 4-1BB binding domains are linked by a peptidyl linker, preferably a proline-threonine (PT) rich linker (such as a linker comprising SEQ ID NO:4) or a GS linker; (viii) the recombinant protein comprises, from the N-terminus to C-terminus: (FAP binding domain)-(linker)-(4-1BB binding domain)-(linker)-(4-1BB binding domain); (ix) the recombinant protein comprises a serum albumin binding domain at the N-terminus, the C-terminus, or both; (x) the recombinant binding protein is capable of binding FAP and 4-1BB simultaneously; (xi) the recombinant protein does not inhibit FAP protease activity, or the reduction in FAP protease activity in the presence of the recombinant protein is no more than 25%, no more than 20%, no more than 15%, or no more than 10%; (xii) the recombinant protein has a terminal half-life in a mouse model of at least 10 hours, at least 20 hours, at least 30 hours, at least 40 hours, or about 44 hours, and (xiii) the recombinant protein has a terminal half-life in a cynomolgus monkey model of at least 1 day, at least 2 days, at least 3 days, at least 4 days, or about 2.8 days, or about 4.5 days.

In certain embodiments, the recombinant protein described here comprises a FAP binding domain and two 4-1BB binding domains, wherein: (a) the FAP binding domain comprises an amino acid sequence that is at least 90% identical to SEQ ID NO: 2; wherein its N-terminus optionally further comprises a G, an S, or a GS; and wherein the second last position can be L or A, and the last position can be N or A; and (b) each of said two 4-1BB binding domains independently comprises an amino acid sequence that is at least 90% identical to SEQ ID NO: 3; wherein its N-terminus optionally further comprises a G, an S, or a GS; and wherein the second last position can be L or A, and the last position can be N or A; and (c) the recombinant protein comprises, from the N-terminus to C-terminus: (FAP binding domain)-(4-1BB binding domain)-(4-1BB binding domain). In certain embodiments, the recombinant protein comprises, from the N-terminus to C-terminus: (FAP binding domain)-(linker)-(4-1BB binding domain)-(linker)-(4-1BB binding domain). In certain embodiments, the linker comprises SEQ ID NO:4. In certain embodiments, the recombinant protein binds human 4-1BB in PBS with a dissociation constant (K_{D}) value below 10⁻⁷M, below 10⁻⁸M, below 5×10⁻⁹M, or below 3×10⁻⁹M; and/or the recombinant protein binds human FAP in PBS with a dissociation constant (K_{D}) value below 10⁻⁸M, below 5×10⁻⁹M, below 3×10⁻⁹M, or below 1×10⁻⁹M. The recombinant protein may further comprise a serum albumin binding domain at the N-terminus, the C-terminus, or both.

In certain embodiments, the recombinant protein described here comprises a FAP binding domain and two 4-1BB binding domains, wherein: (a) the FAP binding domain comprises an amino acid sequence that is at least 95% identical to SEQ ID NO: 2; wherein its N-terminus optionally further comprises a G, an S, or a GS; and wherein the second last position can be L or A, and the last position can be N or A; and (b) each of said two 4-1BB binding domains independently comprises an amino acid sequence that is at least 95% identical to SEQ ID NO: 3; wherein its N-terminus optionally further comprises a G, an S, or a GS; and wherein the second last position can be L or A, and the last position can be N or A; and (c) the recombinant protein comprises, from the N-terminus to C-terminus: (FAP binding domain)-(4-1BB binding domain)-(4-1BB binding domain). In certain embodiments, the recombinant protein comprises, from the N-terminus to C-terminus: (FAP binding domain)-(linker)-(4-1BB binding domain)-(linker)-(4-1BB binding domain). In certain embodiments, the linker comprises SEQ ID NO:4. In certain embodiments, the recombinant protein binds human 4-1BB in PBS with a dissociation constant (K_{D}) value below 10⁻⁷M, below 10⁻⁸M, below 5×10⁻⁹M, or below 3×10⁻⁹M; and/or the recombinant protein binds human FAP in PBS with a dissociation constant (K_{D}) value below 10⁻⁸M, below 5×10⁻⁹M, below 3×10⁻⁹M, or below 1×10⁻⁹M. The recombinant protein may further comprise a serum albumin binding domain at the N-terminus, the C-terminus, or both.

In certain embodiments, the recombinant protein described here comprises a FAP binding domain and two 4-1BB binding domains, wherein: (a) the FAP binding domain comprises an amino acid sequence that is at least 98% identical to SEQ ID NO: 2; wherein its N-terminus optionally further comprises a G, an S, or a GS; and wherein the second last position can be L or A, and the last position can be N or A; and (b) each of said two 4-1BB binding domains independently comprises an amino acid sequence that is at least 98% identical to SEQ ID NO: 3; wherein its N-terminus optionally further comprises a G, an S, or a GS; and wherein the second last position can be L or A, and the last position can be N or A; and (c) the recombinant protein comprises, from the N-terminus to C-terminus: (FAP binding domain)-(4-1BB binding domain)-(4-1BB binding domain). In certain embodiments, the recombinant protein comprises, from the N-terminus to C-terminus: (FAP binding domain)-(linker)-(4-1BB binding domain)-(linker)-(4-1BB binding domain). In certain embodiments, the linker comprises SEQ ID NO:4. In certain embodiments, the recombinant protein binds human 4-1BB in PBS with a dissociation constant (K_{D}) value below 10⁻⁷M, below 10⁻⁸M, below 5×10⁻⁹M, or below 3×10⁻⁹M; and/or the recombinant protein binds human FAP in PBS with a dissociation constant (K_{D}) value below 10⁻⁸M, below 5×10⁻⁹M, below 3×10⁻⁹M, or below 1×10⁻⁹M. The recombinant protein may further comprise a serum albumin binding domain at the N-terminus, the C-terminus, or both.

In certain embodiments, the recombinant protein described here comprises a FAP binding domain and two 4-1BB binding domains, wherein: (a) the FAP binding domain comprises the amino acid sequence of SEQ ID NO: 2; wherein its N-terminus optionally further comprises a G, an S, or a GS; and wherein the second last position can be L or A, and the last position can be N or A; and (b) each of said two 4-1BB binding domains independently comprises the amino acid sequence of SEQ ID NO: 3; wherein its N-terminus optionally further comprises a G, an S, or a GS; and wherein the second last position can be L or A, and the last position can be N or A; and (c) the recombinant protein comprises, from the N-terminus to C-terminus: (FAP binding domain)-(4-1BB binding domain)-(4-1BB binding domain). In certain embodiments, the recombinant protein comprises, from the N-terminus to C-terminus: (FAP binding domain)-(linker)-(4-1BB binding domain)-(linker)-(4-1BB binding domain). In certain embodiments, the linker comprises SEQ ID NO:4. In certain embodiments, the recombinant protein binds human 4-1BB in PBS with a dissociation constant (K_{D}) value below 10⁻⁷M, below 10⁻⁸M, below 5×10⁻⁹M, or below 3×10⁻⁹M; and/or the recombinant protein binds human FAP in PBS with a dissociation constant (K_{D}) value below 10⁻⁸M, below 5×10⁻⁹M, below 3×10⁻⁹M, or below 1×10⁻⁹M. The recombinant protein may further comprise a serum albumin binding domain at the N-terminus, the C-terminus, or both.

In certain embodiments, the recombinant protein described here comprises a first ankyrin repeat domain that specifically binds human FAP, a second ankyrin repeat domain that specifically binds human 4-1BB, a third ankyrin repeat domain that specifically binds human 4-1BB, a fourth ankyrin repeat domain that specifically binds human serum albumin, and a fifth ankyrin repeat domain that specifically binds human serum albumin, wherein said ankyrin repeat domains are arranged, from the N-terminus to C-terminus, according to the following formula: (serum albumin binding domain)-(linker)-(FAP-binding domain)-(linker)-(4-1BB binding domain)-(linker)-(4-1BB binding domain)-(linker)-(serum albumin binding domain), and wherein said recombinant protein specifically binds human FAP in PBS with a dissociation constant (K_{D}) value below 10⁻⁹M, and wherein said recombinant protein specifically binds human 4-1BB in PBS with a dissociation constant (K_{D}) value below 10⁻⁹M or below 10⁻¹⁰M, and wherein said recombinant protein specifically binds human serum albumin in PBS with a dissociation constant (K_{D}) value below 10⁻⁷M or below 10⁻⁸M, and wherein said FAP binding domain comprises an amino acid sequence that is at least 90% identical to SEQ ID NO: 2; wherein its N-terminus optionally further comprises a G, an S, or a GS, and wherein optionally A at the second last position is substituted with L and/or A at the last position is substituted with N, and wherein each of said two 4-1BB binding domains independently comprises an amino acid sequence that is at least 90% identical to SEQ ID NO: 3; wherein its N-terminus optionally further comprises a G, an S, or a GS, and wherein optionally A at the second last position is substituted with L and/or A at the last position is substituted with N. In certain embodiments, the linker comprises SEQ ID NO:4. In certain embodiments, the linker consists of SEQ ID NO:4. In certain embodiments, said recombinant protein simultaneously binds to human 4-1BB, human FAP and human serum albumin; wherein preferably said simultaneous binding is measured by surface plasmon resonance (SPR), further preferably as described in Example 3. In certain embodiments, each of said two serum albumin binding domains independently comprises an amino acid sequence that is at least 90% identical to SEQ ID NO: 1, wherein its N-terminus optionally further comprises a G, an S, or a GS, and wherein optionally A at the second last position is substituted with L and/or A at the last position is substituted with N.

In certain embodiments, the recombinant protein described here comprises a first ankyrin repeat domain that specifically binds human FAP, a second ankyrin repeat domain that specifically binds human 4-1BB, a third ankyrin repeat domain that specifically binds human 4-1BB, a fourth ankyrin repeat domain that specifically binds human serum albumin, and a fifth ankyrin repeat domain that specifically binds human serum albumin, wherein said ankyrin repeat domains are arranged, from the N-terminus to C-terminus, according to the following formula: (serum albumin binding domain)-(linker)-(FAP-binding domain)-(linker)-(4-1BB binding domain)-(linker)-(4-1BB binding domain)-(linker)-(serum albumin binding domain), and wherein said recombinant protein specifically binds human FAP in PBS with a dissociation constant (K_{D}) value below 10⁻⁹M, and wherein said recombinant protein specifically binds human 4-1BB in PBS with a dissociation constant (K_{D}) below 10⁻⁹M or below 10⁻¹⁰M, and wherein said recombinant protein specifically binds human serum albumin in PBS with a dissociation constant (K_{D}) value below 10⁻⁷M or below 10⁻⁸M, and wherein said FAP binding domain comprises an amino acid sequence that is at least 93% identical to SEQ ID NO: 2; wherein its N-terminus optionally further comprises a G, an S, or a GS, and wherein optionally A at the second last position is substituted with L and/or A at the last position is substituted with N, and wherein each of said two 4-1BB binding domains independently comprises an amino acid sequence that is at least 93% identical to SEQ ID NO: 3; wherein its N-terminus optionally further comprises a G, an S, or a GS, and wherein optionally A at the second last position is substituted with L and/or A at the last position is substituted with N. In certain embodiments, the linker comprises SEQ ID NO:4. In certain embodiments, the linker consists of SEQ ID NO:4. In certain embodiments, said recombinant protein simultaneously binds to human 4-1BB, human FAP and human serum albumin; wherein preferably said simultaneous binding is measured by surface plasmon resonance (SPR), further preferably as described in Example 3. In certain embodiments, each of said two serum albumin binding domains independently comprises an amino acid sequence that is at least 93% identical to SEQ ID NO: 1, wherein its N-terminus optionally further comprises a G, an S, or a GS, and wherein optionally A at the second last position is substituted with L and/or A at the last position is substituted with N.

In certain embodiments, the recombinant protein described here comprises a first ankyrin repeat domain that specifically binds human FAP, a second ankyrin repeat domain that specifically binds human 4-1BB, a third ankyrin repeat domain that specifically binds human 4-1BB, a fourth ankyrin repeat domain that specifically binds human serum albumin, and a fifth ankyrin repeat domain that specifically binds human serum albumin, wherein said ankyrin repeat domains are arranged, from the N-terminus to C-terminus, according to the following formula: (serum albumin binding domain)-(linker)-(FAP-binding domain)-(linker)-(4-1BB binding domain)-(linker)-(4-1BB binding domain)-(linker)-(serum albumin binding domain), and wherein said recombinant protein specifically binds human FAP in PBS with a dissociation constant (K_{D}) value below 10⁻⁹M, and wherein said recombinant protein specifically binds human 4-1BB in PBS with a dissociation constant (K_{D}) below 10⁻⁹M or below 10⁻¹⁰M, and wherein said recombinant protein specifically binds human serum albumin in PBS with a dissociation constant (K_{D}) value below 10⁻⁷M or below 10⁻⁸M, and wherein said FAP binding domain comprises an amino acid sequence that is at least 95% identical to SEQ ID NO: 2; wherein its N-terminus optionally further comprises a G, an S, or a GS, and wherein optionally A at the second last position is substituted with L and/or A at the last position is substituted with N, and wherein each of said two 4-1BB binding domains independently comprises an amino acid sequence that is at least 95% identical to SEQ ID NO: 3; wherein its N-terminus optionally further comprises a G, an S, or a GS, and wherein optionally A at the second last position is substituted with L and/or A at the last position is substituted with N. In certain embodiments, the linker comprises SEQ ID NO:4. In certain embodiments, the linker consists of SEQ ID NO:4. In certain embodiments, said recombinant protein simultaneously binds to human 4-1BB, human FAP and human serum albumin; wherein preferably said simultaneous binding is measured by surface plasmon resonance (SPR), further preferably as described in Example 3. In certain embodiments, each of said two serum albumin binding domains independently comprises an amino acid sequence that is at least 95% identical to SEQ ID NO: 1, wherein its N-terminus optionally further comprises a G, an S, or a GS, and wherein optionally A at the second last position is substituted with L and/or A at the last position is substituted with N.

In certain embodiments, the recombinant protein described here comprises a first ankyrin repeat domain that specifically binds human FAP, a second ankyrin repeat domain that specifically binds human 4-1BB, a third ankyrin repeat domain that specifically binds human 4-1BB, a fourth ankyrin repeat domain that specifically binds human serum albumin, and a fifth ankyrin repeat domain that specifically binds human serum albumin, wherein said ankyrin repeat domains are arranged, from the N-terminus to C-terminus, according to the following formula: (serum albumin binding domain)-(linker)-(FAP-binding domain)-(linker)-(4-1BB binding domain)-(linker)-(4-1BB binding domain)-(linker)-(serum albumin binding domain), and wherein said recombinant protein specifically binds human FAP in PBS with a dissociation constant (K_{D}) value below 10⁻⁹M, and wherein said recombinant protein specifically binds human 4-1BB in PBS with a dissociation constant (K_{D}) value below 10⁻⁹M or below 10⁻¹⁰M, and wherein said recombinant protein specifically binds human serum albumin in PBS with a dissociation constant (K_{D}) value below 10⁻⁷M or below 10⁻⁸M, and wherein said FAP binding domain comprises an amino acid sequence that is at least 98% identical to SEQ ID NO: 2; wherein its N-terminus optionally further comprises a G, an S, or a GS, and wherein optionally A at the second last position is substituted with L and/or A at the last position is substituted with N, and wherein each of said two 4-1BB binding domains independently comprises an amino acid sequence that is at least 98% identical to SEQ ID NO: 3; wherein its N-terminus optionally further comprises a G, an S, or a GS, and wherein optionally A at the second last position is substituted with L and/or A at the last position is substituted with N. In certain embodiments, the linker comprises SEQ ID NO:4. In certain embodiments, the linker consists of SEQ ID NO:4. In certain embodiments, said recombinant protein simultaneously binds to human 4-1BB, human FAP and human serum albumin; wherein preferably said simultaneous binding is measured by surface plasmon resonance (SPR), further preferably as described in Example 3. In certain embodiments, each of said two serum albumin binding domains independently comprises an amino acid sequence that is at least 98% identical to SEQ ID NO: 1, wherein its N-terminus optionally further comprises a G, an S, or a GS, and wherein optionally A at the second last position is substituted with L and/or A at the last position is substituted with N.

In certain embodiments, the recombinant protein described here comprises a first ankyrin repeat domain that specifically binds human FAP, a second ankyrin repeat domain that specifically binds human 4-1BB, a third ankyrin repeat domain that specifically binds human 4-1BB, a fourth ankyrin repeat domain that specifically binds human serum albumin, and a fifth ankyrin repeat domain that specifically binds human serum albumin, wherein said ankyrin repeat domains are arranged, from the N-terminus to C-terminus, according to the following formula: (serum albumin binding domain)-(linker)-(FAP-binding domain)-(linker)-(4-1BB binding domain)-(linker)-(4-1BB binding domain)-(linker)-(serum albumin binding domain), and wherein said recombinant protein specifically binds human FAP in PBS with a dissociation constant (K_{D}) value below 10⁻⁹M, and wherein said recombinant protein specifically binds human 4-1BB in PBS with a dissociation constant (K_{D}) value below 10⁻⁹M or below 10⁻¹⁰M, and wherein said recombinant protein specifically binds human serum albumin in PBS with a dissociation constant (K_{D}) value below 10⁻⁷M or below 10⁻⁸M, and wherein said FAP binding domain comprises the amino acid sequence of SEQ ID NO: 2; wherein its N-terminus optionally further comprises a G, an S, or a GS, and wherein optionally A at the second last position is substituted with L and/or A at the last position is substituted with N, and wherein each of said two 4-1BB binding domains independently comprises the amino acid sequence of SEQ ID NO: 3; wherein its N-terminus optionally further comprises a G, an S, or a GS, and wherein optionally A at the second last position is substituted with L and/or A at the last position is substituted with N. In certain embodiments, the linker comprises SEQ ID NO:4. In certain embodiments, the linker consists of SEQ ID NO:4. In certain embodiments, said recombinant protein simultaneously binds to human 4-1BB, human FAP and human serum albumin; wherein preferably said simultaneous binding is measured by surface plasmon resonance (SPR), further preferably as described in Example 3. In certain embodiments, each of said two serum albumin binding domains independently comprises the amino acid sequence of SEQ ID NO: 1, wherein its N-terminus optionally further comprises a G, an S, or a GS, and wherein optionally A at the second last position is substituted with L and/or A at the last position is substituted with N.

In certain embodiments, the recombinant protein described here comprises a first ankyrin repeat domain that specifically binds human FAP, a second ankyrin repeat domain that specifically binds human 4-1BB, a third ankyrin repeat domain that specifically binds human 4-1BB, a fourth ankyrin repeat domain that specifically binds human serum albumin, and a fifth ankyrin repeat domain that specifically binds human serum albumin, wherein said ankyrin repeat domains are arranged, from the N-terminus to C-terminus, according to the following formula: (serum albumin binding domain)-(linker)-(FAP-binding domain)-(linker)-(4-1BB binding domain)-(linker)-(4-1BB binding domain)-(linker)-(serum albumin binding domain), and wherein said recombinant protein specifically binds human FAP in PBS with a dissociation constant (K_{D}) value below 10⁻⁹M, and wherein said recombinant protein specifically binds human 4-1BB in PBS with a dissociation constant (K_{D}) below 10⁻⁹M or below 10⁻¹⁰M, and wherein said recombinant protein specifically binds human serum albumin in PBS with a dissociation constant (K_{D}) value below 10⁻⁷M or below 10⁻⁸M, and wherein said FAP binding domain comprises an amino acid sequence that is at least 90% identical to SEQ ID NO: 2; wherein its N-terminus optionally further comprises a G, an S, or a GS, and wherein optionally A at the second last position is substituted with L and/or A at the last position is substituted with N, and wherein each of said two 4-1BB binding domains independently comprises an amino acid sequence that is at least 90% identical to SEQ ID NO: 3; wherein its N-terminus optionally further comprises a G, an S, or a GS, and wherein optionally A at the second last position is substituted with L and/or A at the last position is substituted with N, and wherein each of said two serum albumin binding domains independently comprises an amino acid sequence that is at least 90% identical to SEQ ID NO: 1, wherein its N-terminus optionally further comprises a G, an S, or a GS, and wherein optionally A at the second last position is substituted with L and/or A at the last position is substituted with N, and wherein said recombinant protein is capable of binding simultaneously to human FAP, human 4-1BB and human serum albumin. In certain embodiments, the linker comprises SEQ ID NO:4. In certain embodiments, the linker consists of SEQ ID NO:4. In certain embodiments, the recombinant protein activates human 4-1BB in 4-1BB-expressing HT1080 cells in the presence of FAP-expressing CHO cells with an EC₅₀ value of about 10⁻⁸M or less, or about 10⁻⁹M or less. Simultaneous binding to human 4-1BB, human FAP and human serum albumin is preferably measured in PBS by surface plasmon resonance (SPR), further preferably as described in Example 3.

In certain embodiments, the recombinant protein described here comprises a first ankyrin repeat domain that specifically binds human FAP, a second ankyrin repeat domain that specifically binds human 4-1BB, a third ankyrin repeat domain that specifically binds human 4-1BB, a fourth ankyrin repeat domain that specifically binds human serum albumin, and a fifth ankyrin repeat domain that specifically binds human serum albumin, wherein said ankyrin repeat domains are arranged, from the N-terminus to C-terminus, according to the following formula: (serum albumin binding domain)-(linker)-(FAP-binding domain)-(linker)-(4-1BB binding domain)-(linker)-(4-1BB binding domain)-(linker)-(serum albumin binding domain), and wherein said recombinant protein specifically binds human FAP in PBS with a dissociation constant (K_{D}) value below 10⁻⁹M, and wherein said recombinant protein specifically binds human 4-1BB in PBS with a dissociation constant (K_{D}) value below 10⁻⁹M or below 10⁻¹⁰M, and wherein said recombinant protein specifically binds human serum albumin in PBS with a dissociation constant (K_{D}) value below 10⁻⁷M or below 10⁻⁸M, and wherein said FAP binding domain comprises an amino acid sequence that is at least 95% identical to SEQ ID NO: 2; wherein its N-terminus optionally further comprises a G, an S, or a GS, and wherein optionally A at the second last position is substituted with L and/or A at the last position is substituted with N, and wherein each of said two 4-1BB binding domains independently comprises an amino acid sequence that is at least 95% identical to SEQ ID NO: 3; wherein its N-terminus optionally further comprises a G, an S, or a GS, and wherein optionally A at the second last position is substituted with L and/or A at the last position is substituted with N, and wherein each of said two serum albumin binding domains independently comprises an amino acid sequence that is at least 95% identical to SEQ ID NO: 1, wherein its N-terminus optionally further comprises a G, an S, or a GS, and wherein optionally A at the second last position is substituted with L and/or A at the last position is substituted with N, and wherein said recombinant protein is capable of binding simultaneously to human FAP, human 4-1BB and human serum albumin. In certain embodiments, the linker comprises SEQ ID NO:4. In certain embodiments, the linker consists of SEQ ID NO:4. In certain embodiments, the recombinant protein activates human 4-1BB in 4-1BB-expressing HT1080 cells in the presence of FAP-expressing CHO cells with an EC₅₀ value of about 10⁻⁸M or less, or about 10⁻⁹M or less. Simultaneous binding to human 4-1BB, human FAP and human serum albumin is preferably measured in PBS by surface plasmon resonance (SPR), further preferably as described in Example 3.

In certain embodiments, the recombinant protein described here comprises a first ankyrin repeat domain that specifically binds human FAP, a second ankyrin repeat domain that specifically binds human 4-1BB, a third ankyrin repeat domain that specifically binds human 4-1BB, a fourth ankyrin repeat domain that specifically binds human serum albumin, and a fifth ankyrin repeat domain that specifically binds human serum albumin, wherein said ankyrin repeat domains are arranged, from the N-terminus to C-terminus, according to the following formula: (serum albumin binding domain)-(linker)-(FAP-binding domain)-(linker)-(4-1BB binding domain)-(linker)-(4-1BB binding domain)-(linker)-(serum albumin binding domain), and wherein said recombinant protein specifically binds human FAP in PBS with a dissociation constant (K_{D}) value below 10⁻⁹M, and wherein said recombinant protein specifically binds human 4-1BB in PBS with a dissociation constant (K_{D}) value below 10⁻⁹M or below 10⁻¹⁰M, and wherein said recombinant protein specifically binds human serum albumin in PBS with a dissociation constant (K_{D}) value below 10⁻⁷M or below 10⁻⁸M, and wherein said FAP binding domain comprises the amino acid sequence of SEQ ID NO: 2; wherein its N-terminus optionally further comprises a G, an S, or a GS, and wherein optionally A at the second last position is substituted with L and/or A at the last position is substituted with N, and wherein each of said two 4-1BB binding domains independently comprises the amino acid sequence of SEQ ID NO: 3; wherein its N-terminus optionally further comprises a G, an S, or a GS, and wherein optionally A at the second last position is substituted with L and/or A at the last position is substituted with N, and wherein each of said two serum albumin binding domains independently comprises the amino acid sequence of SEQ ID NO: 1, wherein its N-terminus optionally further comprises a G, an S, or a GS, and wherein optionally A at the second last position is substituted with L and/or A at the last position is substituted with N, and wherein said recombinant protein is capable of binding simultaneously to human FAP, human 4-1BB and human serum albumin. In certain embodiments, the linker comprises SEQ ID NO:4. In certain embodiments, the linker consists of SEQ ID NO:4. In certain embodiments, the recombinant protein activates human 4-1BB in 4-1BB-expressing HT1080 cells in the presence of FAP-expressing CHO cells with an EC₅₀ value of about 10⁻⁸M or less, or about 10⁻⁹M or less. Simultaneous binding to human 4-1BB, human FAP and human serum albumin is preferably measured in PBS by surface plasmon resonance (SPR), further preferably as described in Example 3.

In certain embodiments, the recombinant protein described here comprises a first ankyrin repeat domain that specifically binds human FAP, a second ankyrin repeat domain that specifically binds human 4-1BB, a third ankyrin repeat domain that specifically binds human 4-1BB, a fourth ankyrin repeat domain that specifically binds human serum albumin, and a fifth ankyrin repeat domain that specifically binds human serum albumin, wherein said ankyrin repeat domains are arranged, from the N-terminus to C-terminus, according to the following formula: (serum albumin binding domain)-(linker)-(FAP-binding domain)-(linker)-(4-1BB binding domain)-(linker)-(4-1BB binding domain)-(linker)-(serum albumin binding domain), and wherein said recombinant protein specifically binds human FAP in PBS with a dissociation constant (K_{D}) value below 10⁻⁹M, and wherein said recombinant protein specifically binds human 4-1BB in PBS with a dissociation constant (K_{D}) value below 10⁻⁹M, and wherein said recombinant protein specifically binds human serum albumin in PBS with a dissociation constant (K_{D}) value below 10⁻⁸M, and wherein said FAP binding domain comprises an amino acid sequence that is at least 90% identical to SEQ ID NO: 2; wherein its N-terminus optionally further comprises a G, an S, or a GS, and wherein optionally A at the second last position is substituted with L and/or A at the last position is substituted with N, and wherein each of said two 4-1BB binding domains independently comprises an amino acid sequence that is at least 90% identical to SEQ ID NO: 3; wherein its N-terminus optionally further comprises a G, an S, or a GS, and wherein optionally A at the second last position is substituted with L and/or A at the last position is substituted with N, and wherein each of said two serum albumin binding domains independently comprises an amino acid sequence that is at least 90% identical to SEQ ID NO: 1, wherein its N-terminus optionally further comprises a G, an S, or a GS, and wherein optionally A at the second last position is substituted with L and/or A at the last position is substituted with N, and wherein said recombinant protein is capable of binding simultaneously to human FAP, human 4-1BB and human serum albumin. In certain embodiments, the linker comprises SEQ ID NO:4. In certain embodiments, the linker consists of SEQ ID NO:4. In certain embodiments, the recombinant protein activates human 4-1BB in 4-1BB-expressing HT1080 cells in the presence of FAP-expressing CHO cells with an EC₅₀ value of about 10⁻⁸M or less, or about 10⁻⁹M or less. Simultaneous binding to human 4-1BB, human FAP and human serum albumin is preferably measured in PBS by surface plasmon resonance (SPR), further preferably as described in Example 3.

3.8. Nucleic Acids and Methods of Producing Multispecific Proteins

The disclosure also provides polynucleotides encoding the recombinant proteins described herein. The disclosure also provides a method of making any of the polynucleotides described herein. Polynucleotides can be made and expressed by procedures known in the art.

In one aspect, the disclosure provides polynucleotides or compositions comprising polynucleotides encoding a recombinant multispecific protein, wherein sad protein comprises a first ankyrin repeat domain that specifically binds fibroblast activation protein (FAP), and a second ankyrin repeat domain that specifically binds 4-1BB, and optionally, a half-life extending moiety.

In one aspect, the disclosure provides polynucleotides or compositions comprising polynucleotides comprising a nucleic acid sequence encoding a recombinant protein comprising SEQ ID NO:1, 2, 3, 4, or 5. In one aspect, the disclosure provides polynucleotides or compositions comprising polynucleotides comprising a nucleic acid sequence encoding a recombinant protein comprising SEQ ID NO:6. In one embodiment, the disclosure provides a nucleic acid comprising the nucleic acid sequence of SEQ ID NO: 17.

In another aspect, the disclosure provides polynucleotides and variants thereof encoding a recombinant protein, wherein such variant polynucleotides share at least 70%, at least 75%, at least 80%, at least 85%, at least 87%, at least 89%, at least 90%, at least 91%, at least 92%, at least 93%, at least 94%, at least 95%, at least 96%, at least 97%, at least 98%, or at least 99% sequence identity to any nucleic acid disclosed herein, such as a nucleic acid comprising the nucleic acid sequence of SEQ ID NO: 17. In some embodiments, such variant polynucleotides share at least 95%, sequence identity to any nucleic acid disclosed herein, such as a nucleic acid comprising the nucleic acid sequence of SEQ ID NO: 17. In some embodiments, such variant polynucleotides share at least 96%, sequence identity to any nucleic acid disclosed herein such as a nucleic acid sequence comprising the nucleic acid of SEQ ID NO: 17. In some embodiments, such variant polynucleotides share at least 97%, sequence identity to any nucleic acid disclosed herein, such as a nucleic acid comprising the nucleic acid sequence of SEQ ID NO: 17. In some embodiments, such variant polynucleotides share at least 98%, sequence identity to any nucleic acid disclosed herein, such as a nucleic acid comprising the nucleic acid sequence of SEQ ID NO: 17. In some embodiments, such variant polynucleotides share at least 99%, sequence identity to any nucleic acid disclosed herein, such as a nucleic acid comprising the nucleic acid sequence of SEQ ID NO: 17.

In another aspect, the disclosure provides polynucleotides and variants thereof encoding a recombinant protein, wherein such variant polynucleotides are capable of hybridizing under highly stringent conditions to the sequence of SEQ ID NO: 17. “Highly stringent conditions” includes those that: (1) employ low ionic strength and high temperature for washing, for example 0.015 M sodium chloride/0.0015 M sodium citrate/0.1% sodium dodecyl sulfate at 50° C.; (2) employ a denaturing agent during hybridization, such as formamide, for example, 50% (v/v) formamide with 0.1% bovine serum albumin/0.1% Ficoll/0.1% polyvinylpyrrolidone/50 mM sodium phosphate buffer at pH 6.5 with 750 mM sodium chloride, 75 mM sodium citrate at 42° C.; or (3) employ 50% formamide, 5×SSC (0.75 M NaCl, 0.075 M sodium citrate), 50 mM sodium phosphate (pH 6.8), 0.1% sodium pyrophosphate, 5×Denhardt's solution, sonicated salmon sperm DNA (50 pg/mL), 0.1% SDS, and 10% dextran sulfate at 42° C., with washes at 42° C. in 0.2×SSC (sodium chloride/sodium citrate) and 50% formamide at 55° C., followed by a high-stringency wash consisting of 0.1×SSC containing EDTA at 55° C.

Polynucleotides complementary to any such sequences are also encompassed by the present disclosure. Polynucleotides may be single-stranded (coding or antisense) or double-stranded, and may be DNA (recombinant, cDNA or synthetic) or RNA molecules. RNA molecules include hnRNA molecules, which contain introns and correspond to a DNA molecule in a one-to-one manner, and mRNA molecules, which do not contain introns. Additional coding or non-coding sequences may, but need not, be present within a polynucleotide of the present disclosure, and a polynucleotide may, but need not, be linked to other molecules and/or support materials.

It will be appreciated by those of ordinary skill in the art that, as a result of the degeneracy of the genetic code, there are many nucleotide sequences that encode a recombinant protein (or its individual domains) comprising an amino acid sequence as described herein. Some of these polynucleotides bear minimal homology to the nucleotide sequence of any native gene. Polynucleotides that vary due to differences in codon usage are specifically contemplated by the present disclosure.

The present disclosure also includes codon-optimized polynucleotides wherein the nucleic acid sequence has been optimized to maximize expression in a particular cell. In general, codon optimization refers to a process of modifying a nucleic acid sequence for enhanced expression in the host cells of interest by replacing at least one codon (e.g., about or more than about 1, 2, 3, 4, 5, 10, 15, 20, 25, 50, or more codons) of the original sequence with codons that are more frequently or most frequently used in the genes of that host cell while maintaining the original amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. Codon usage tables are readily available, and these tables can be adapted in a number of ways (e.g., Nakamura, Y., et al. “Codon usage tabulated from the international DNA sequence databases: status for the year 2000” Nucl. Acids Res. 28:292 (2000)). Computer algorithms for codon optimizing a particular sequence for expression in a particular host cell are also available, such as Gene Forge (Aptagen; Jacobus, Pa.), are also available. In some embodiments, one or more codons (e.g., 1, 2, 3, 4, 5, 10, 15, 20, 25, 50, or more, or all codons) in a sequence encoding a recombinant protein correspond to the most frequently used codon for a particular amino acid.

Suitable cloning vectors may be constructed according to standard techniques, or may be selected from a large number of cloning vectors available in the art. While the cloning vector selected may vary according to the host cell intended to be used, useful cloning vectors will generally have the ability to self-replicate, may possess a single target for a particular restriction endonuclease, and/or may carry genes for a marker that can be used in selecting clones containing the vector. Suitable examples include plasmids and bacterial viruses, e.g., pUC18, pUC19, Bluescript (e.g., pBS SK+) and its derivatives, mp18, mp19, pBR322, pMB9, ColE1, pCR1, RP4, phage DNAs, and shuttle vectors such as pSA3 and pAT28. These and many other cloning vectors are available from commercial vendors such as BioRad, Strategene, and Invitrogen.

Expression vectors are further provided. Expression vectors generally are replicable polynucleotide constructs that contain a polynucleotide according to the disclosure. It is implied that an expression vector must be replicable in the host cells either as episomes or as an integral part of the chromosomal DNA. Suitable expression vectors include but are not limited to plasmids, viral vectors, including adenoviruses, adeno-associated viruses, retroviruses, cosmids, and expression vector(s) disclosed in PCT Publication No. WO 87/04462. Vector components may generally include, but are not limited to, one or more of the following: a signal sequence; an origin of replication; one or more marker genes; suitable transcriptional controlling elements (such as promoters, enhancers and terminator). For expression (i.e., translation), one or more translational controlling elements are also usually required, such as ribosome binding sites, translation initiation sites, and stop codons.

The vectors containing the polynucleotides of interest and/or the polynucleotides themselves, can be introduced into the host cell by any of a number of appropriate means, including electroporation, transfection employing calcium chloride, rubidium chloride, calcium phosphate, DEAE-dextran, or other substances; microprojectile bombardment; lipofection; and infection (e.g., where the vector is an infectious agent such as vaccinia virus). The choice of introducing vectors or polynucleotides will often depend on features of the host cell.

Exemplary host cells include an E. coli cell, a yeast cell, an insect cell, a simian COS cell, a Chinese hamster ovary (CHO) cell, or a myeloma cell. Preferred host cells include an E. coli cell, a CHO cell, a Human embryonic kidney (HEK) 293 cell, or a Sp2.0 cell, among many cells well-known in the art.

### 4. Methods of Treatment

The recombinant proteins described herein can be used to, e.g., treat a subject that has cancer.

The disclosure provides a method of treating cancer, comprising administering to a subject in need thereof a therapeutically effective amount of a recombinant protein or a pharmaceutical composition described herein. In certain embodiments, the subject is a human. In certain embodiments, the cancer comprises a solid tumor. In certain embodiments, the cancer cell expresses FAP. In certain embodiments, tumor stromal cells express FAP.

In some embodiments, the cancer is brain cancer, bladder cancer, breast cancer, clear cell kidney cancer, cervical cancer, colon and rectal cancer, endometrial cancer, gastric cancer, head/neck squamous cell carcinoma, lip and oral cancer, liver cancer, lung squamous cell carcinoma, melanoma, mesothelioma, non-small-cell lung cancer (NSCLC), non-melanoma skin cancer, ovarian cancer, oral cancer, pancreatic cancer, prostate cancer, renal cell carcinoma, sarcoma, small-cell lung cancer (SCLC), Squamous Cell Carcinoma of the Head and Neck (SCCHN), triple negative breast cancer, or thyroid cancer.

In some embodiments, the cancer is cancer is adrenocortical tumor, alveolar soft part sarcoma, carcinoma, chondrosarcoma, colorectal carcinoma, desmoid tumors, desmoplastic small round cell tumor, endocrine tumors, endodermal sinus tumor, epithelioid hemangioendothelioma, Ewing sarcoma, germ cell tumor, hepatoblastoma, hepatocellular carcinoma, melanoma, nephroma, neuroblastoma, non-rhabdomyosarcoma soft tissue sarcoma (NRSTS), osteosarcoma, paraspinal sarcoma, renal cell carcinoma, retinoblastoma, rhabdomyosarcoma, synovial sarcoma, or Wilms tumor.

In some embodiments, the cancer is acute lymphoblastic leukemia (ALL), acute myeloid leukemia (AML), chronic lymphocytic leukemia (CLL), or chronic myeloid leukemia (CML).

In some embodiments, the cancer is diffuse large B-cell lymphoma (DLBCL), follicular lymphoma, Hodgkin's lymphoma (HL), mantle cell lymphoma (MCL), multiple myeloma (MM), myelodysplastic syndrome (MDS), non-Hodgkin's lymphoma (NHL), or small lymphocytic lymphoma (SLL).

Indeed, cancers that can be treated include, but are not limited to, alveolar rhabdomyosarcoma, bone cancer, cancer of the anus, anal canal, or anorectum, cancer of the eye, cancer of the intrahepatic bile duct, cancer of the joints, cancer of the neck, gallbladder, or pleura, cancer of the nose, nasal cavity, or middle ear, cancer of the oral cavity, cancer of the vulva, esophageal cancer, gastrointestinal carcinoid tumor, hypopharynx cancer, larynx cancer, nasopharynx cancer, peritoneum, omentum, and mesentery cancer, pharynx cancer, small intestine cancer, soft tissue cancer, stomach cancer, testicular cancer, ureter cancer, and urinary bladder cancer.

In particular aspects, the cancer is selected from the group consisting of: head and neck, ovarian, cervical, bladder and oesophageal cancers, pancreatic, gastrointestinal cancer, gastric, breast, endometrial and colorectal cancers, hepatocellular carcinoma, glioblastoma, bladder, lung cancer, and bronchioloalveolar carcinoma.

In certain embodiments, the cancer is non-small cell lung cancer (NSCLC), head and neck cancer, renal cancer, triple negative breast cancer, or gastric cancer. In certain embodiments, the cancer is non-small cell lung cancer (NSCLC), small cell lung cancer (SCLC), head and neck cancer, renal cancer, breast cancer, melanoma, ovarian cancer, liver cancer, pancreatic cancer, colon cancer, prostate cancer, gastric cancer, lymphoma or leukemia. In certain embodiments, the cancer is brain cancer.

The recombinant proteins described herein may be used prior to or following surgery to remove a tumor and may be used prior to, during or after radiation therapy. The recombinant protein may be used to treat a tumor that is large enough to be found by palpation or by imaging techniques well known in the art, such as MRI, ultrasound, or CAT scan. In some embodiments, the recombinant protein is used to treat an advanced stage tumor having dimensions of at least about 200 mm³, 300 mm³, 400 mm³, 500 mm³, 750 mm³, or up to 1000 mm³.

It has been reported that production of cytokines (IFN-γ, TNF-alpha and IL-2) in serum and cytotoxic T lymphocyte (CTL) activity is indicative of 4-1BB activation (see, e.g., Li et al., Cell Mol Immunol. 2008 October; 5(5):379-84. doi: 10.1038/cmi.2008.47). Therefore, cytokine-related (such as IFN-γ related) expression profile can predict clinical response to 4-1BB activation.

For example, studies have also shown that it IFN-γ can enhance anti-tumor and antiviral effects of CD8+ T cells. The CD8+ T cells are able to produce IFNγ, which enhances their ability to migrate to the site of antigen-presenting cells. Conversely, deprivation of either autocrine or paracrine IFNγ, or blockade of IFNγ signaling to CTL markedly reduced their cytotoxic function, their kinematics, and effector cell survival. The need for local IFNγ to enable cytotoxic CD8+ T-cell function is significant for cancer treatment.

Accordingly, provided herein are methods of increasing T cell activity, in particular CD8+ T-cell mediated cytotoxicity in a subject. Such increase in T cell activity includes, e.g., increasing T cell survival and effector function, restricting terminal differentiation and loss of replicative potential, promoting T cell longevity, and enhancing cytotoxicity against target (e.g., cancer) cells. In certain embodiments, the T cell activity or immune response is directed against a cancer cell or cancer tissue or a tumor cell or tumor. In certain embodiments, the immune response is a humoral immune response. In certain embodiments, the immune response is an innate immune response. In certain embodiments, the immune response which is enhanced is a T-cell mediated immune response.

### 5. Pharmaceutical Compositions and Administration

In another aspect, the disclosure also provides pharmaceutical compositions comprising the recombinant multispecific proteins described herein.

The pharmaceutical compositions may comprise a pharmaceutically acceptable carrier, diluent, or excipient. Standard pharmaceutical carriers include a phosphate buffered saline solution, water, emulsions such as an oil/water or water/oil emulsion, and various types of wetting agents.

The pharmaceutical compositions can comprise any pharmaceutically acceptable ingredients, including, for example, acidifying agents, additives, adsorbents, aerosol propellants, air displacement agents, alkalizing agents, anticaking agents, anticoagulants, antimicrobial preservatives, antioxidants, antiseptics, bases, binders, buffering agents, chelating agents, coating agents, coloring agents, desiccants, detergents, diluents, disinfectants, disintegrants, dispersing agents, dissolution enhancing agents, dyes, emollients, emulsifying agents, emulsion stabilizers, fillers, film forming agents, flavor enhancers, flavoring agents, flow enhancers, gelling agents, granulating agents, humectants, lubricants, mucoadhesives, ointment bases, ointments, oleaginous vehicles, organic bases, pastille bases, pigments, plasticizers, polishing agents, preservatives, sequestering agents, skin penetrants, solubilizing agents, solvents, stabilizing agents, suppository bases, surface active agents, surfactants, suspending agents, sweetening agents, therapeutic agents, thickening agents, tonicity agents, toxicity agents, viscosity-increasing agents, water-absorbing agents, water-miscible cosolvents, water softeners, or wetting agents. See, e.g., the Handbook of Pharmaceutical Excipients, Third Edition, A. H. Kibbe (Pharmaceutical Press, London, UK, 2000), which is incorporated by reference in its entirety. Remington's Pharmaceutical Sciences, Sixteenth Edition, E. W. Martin (Mack Publishing Co., Easton, Pa., 1980), which is incorporated by reference in its entirety.

The pharmaceutical compositions can be formulated to achieve a physiologically compatible pH. In some embodiments, the pH of the pharmaceutical composition can be, for example, between about 4 or about 5 and about 8.0, or between about 4.5 and about 7.5, or between about 5.0 and about 7.5. In exemplary embodiments, the pH of the pharmaceutical composition is between 5.5 and 7.5.

The recombinant multispecific proteins described herein can be administered to the subject via any suitable route of administration, such as parenteral, nasal, oral, pulmonary, topical, vaginal, or rectal administration. Formulations suitable for parenteral administration include aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. For additional details, see Pharmaceutics and Pharmacy Practice, J. B. Lippincott Company, Philadelphia, Pa., Banker and Chalmers, eds., pages 238-250 (1982), and ASHP Handbook on Injectable Drugs, Toissel, 4th ed., pages 622-630 (1986)).

The dose of the active agent of the present disclosure administered over the course of a therapeutic regimen should be sufficient to treat cancer in a clinically-acceptable time frame (e.g., 1 to 4 weeks or longer (such as 5 to 20 or more weeks)) from the time of administration. In certain embodiments, the time period could be even longer. The dose will be determined by the efficacy of the particular active agent and the condition of the animal (e.g., human), as well as, at times, the body weight of the animal (e.g., human) to be treated. The extent to which cancer is treated upon administration of a certain dose can be represented by, for example, the cytotoxicity of the active agent or the extent of tumor regression achieved with the active agent. Methods of measuring cytotoxicity of the recombinant multispecific proteins and methods of assaying tumor regression are known in the art. By way of example and not intending to limit the present disclosure, the dose of the active agent of the present disclosure can be about 0.0001 to about 1 g/kg body weight of the subject being treated/day, from about 0.0001 to about 0.001 g/kg body weight/day, or about 0.01 mg to about 1 g/kg body weight/day. Dosage units may be also expressed in rag/m², which refer to the quantity in milligrams per square meter of body surface area.

Based on PK/PD model and pre-clinical animal model, it is believed that the therapeutic dose can range from about 0.015 mg/kg to about 12 mg/kg, such as from about 0.05 mg/kg to about 10 mg/kg, from about 0.1 mg/kg to about 10 mg/kg, from about 0.5 mg/kg to about 10 mg/kg, from about 0.5 mg/kg to about 7.5 mg/kg, from about 0.5 mg/kg to about 5 mg/kg, about 0.5 mg/kg, about 1.0 mg/kg, about 1.5 mg/kg, about 2.0 mg/kg, about 2.5 mg/kg, about 3.0 mg/kg, about 3.5 mg/kg, about 4.0 mg/kg, about 4.5 mg/kg, or about 5 mg/kg. In certain embodiments, the multispecific protein is administered at from about 0.5 mg/kg to about 5 mg/kg. In certain embodiments, the multispecific protein is administered at about 2 mg/kg.

The recombinant multispecific proteins described herein may be used in combination with another therapeutic agent, such as another anti-cancer agent. Each therapeutic agent may be administered simultaneously (e.g., in the same medicament or at the same time), concurrently (i.e., in separate medicaments administered one right after the other in any order) or sequentially in any order. Sequential administration may be useful when the therapeutic agents in the combination therapy are in different dosage forms (e.g., one agent is a tablet or capsule and another agent is a sterile liquid) and/or are administered on different dosing schedules, e.g., a chemotherapeutic that is administered at least daily and a biotherapeutic that is administered less frequently, such as once weekly, once every two weeks, or once every three weeks.

In certain embodiments, the recombinant multispecific protein described herein is administered about once every week, once every two weeks, once every three weeks, or once a month. For example, the recombinant multispecific protein may be administered at about 0.5 mg/kg, about 1 mg/kg, about 2 mg/kg, about 3 mg/kg, about 4 mg/kg, or about 5 mg/kg every three weeks.

### EXAMPLES

### Example 1—Design of Multispecific Binding Proteins

Multispecific binding proteins in various formats (See FIGS. 5A and 5B) were generated and their ability to enhance NF-κB activation of h4-1BB-HT1080 reporter cells was assessed.

The functional activity of various multispecific binding proteins comprising a FAP binding domain set forth in SEQ ID NO: 2, and one to three 4-1BB binding domains set forth in SEQ ID NO: 3, was assessed using human 4-1BB-transfected NF-κB reporter cells (h4-1BB-HT1080) in the presence of human FAP-expressing CHO cells.

In Vitro NF-κB Activation Assay in the Presence of Human FAP-Expressing CHO Cells:

HT1080 human fibrosarcoma cells were stably transfected with cDNA coding for full-length human 4-1BB and the pNIFTY-Lucia NF-κB reporter gene as described below. In the same way, CHO cells were stably transfected with cDNA coding for human FAP. Using 96-well plates, 40,000 h4-1BB-HT1080 reporter cells and 40,000 CHO-hFAP cells were plated and varying concentrations of MpA or control ankyrin repeat proteins were added to the cells and incubated at 37° C., 5% CO₂. After 20 hours, the supernatant was collected and centrifuged in a fresh 96-well plate. QUANTI-Luc reagent was mixed with the supernatant and luminescence read on a Tecan M1000 luminescence plate reader. EC₅₀ values were determined by fitting the data with the four-parameter logistical fit model using Graphpad Prism software (version 7.02).

Generation of CHO Cells Expressing Human FAP:

CHO cells were stably transfected to express human FAP on the cell surface. A plasmid containing a GFP-fusion of the ORF of human fibroblast activation protein (FAP) was obtained from OriGene Technologies (#RG204692). The cDNA coding for human FAP (without GFP) was sub-cloned using standard molecular biology techniques. This plasmid was then transfected into CHO cells to produce stable transfectants overexpressing human FAP using Lipofectamine. Selection pressure was applied using different amounts of Geneticin G-418 (Promega, V8091). Expression of hFAP was analysed by flow cytometry using the anti-FAP antibody ESC11 (International Patent Publication No. WO2011/040972). The population of CHO-hFAP transfectants from condition 1.9 mg/mL G-418 were chosen for a lower expression level of FAP for the potency assay in order to allow discrimination of high potency DARPin® molecules.

Conclusion:

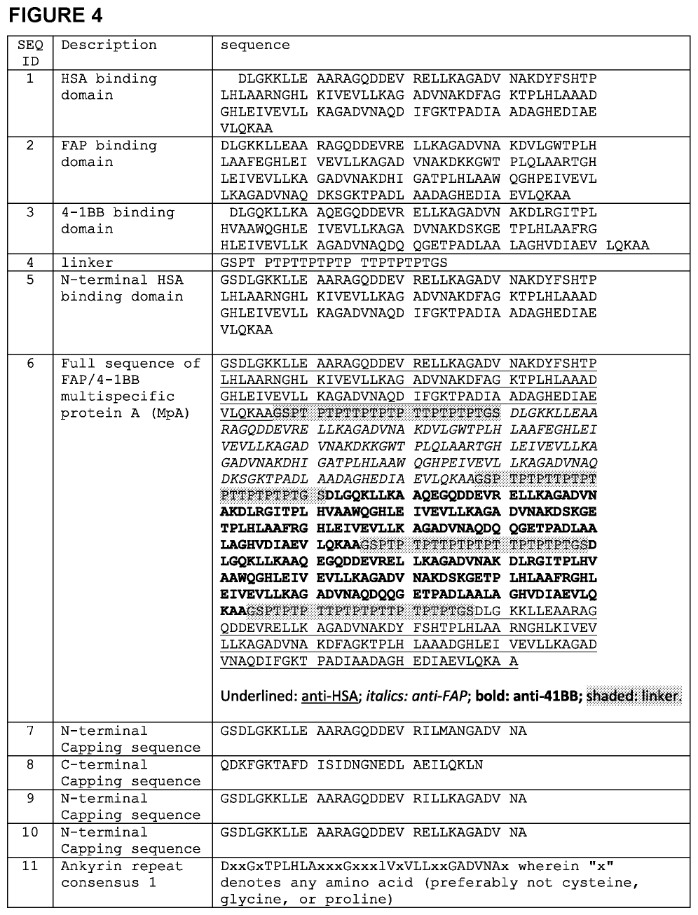

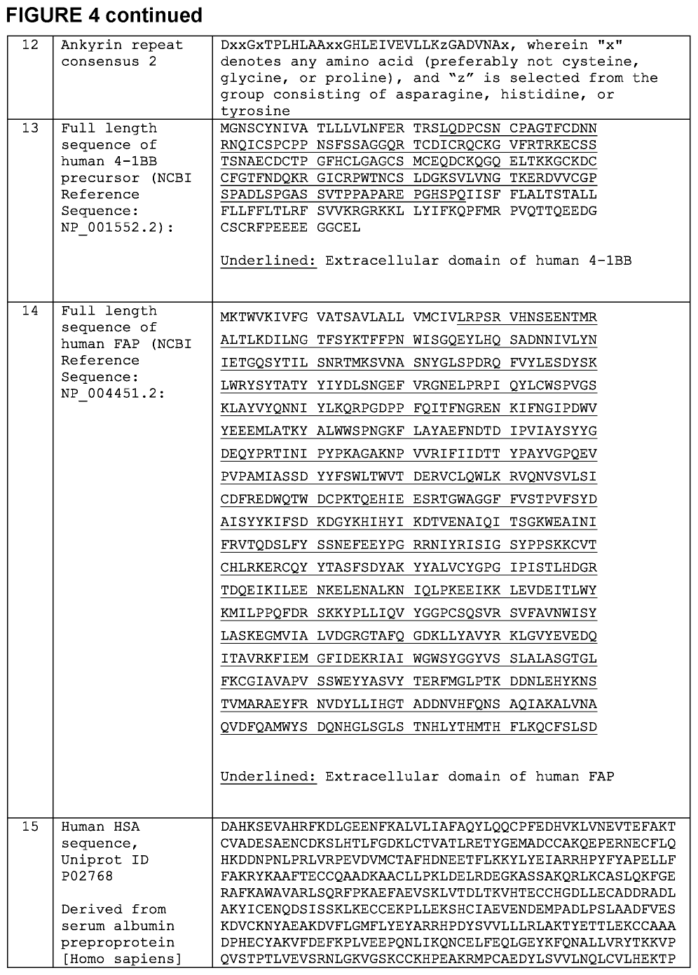

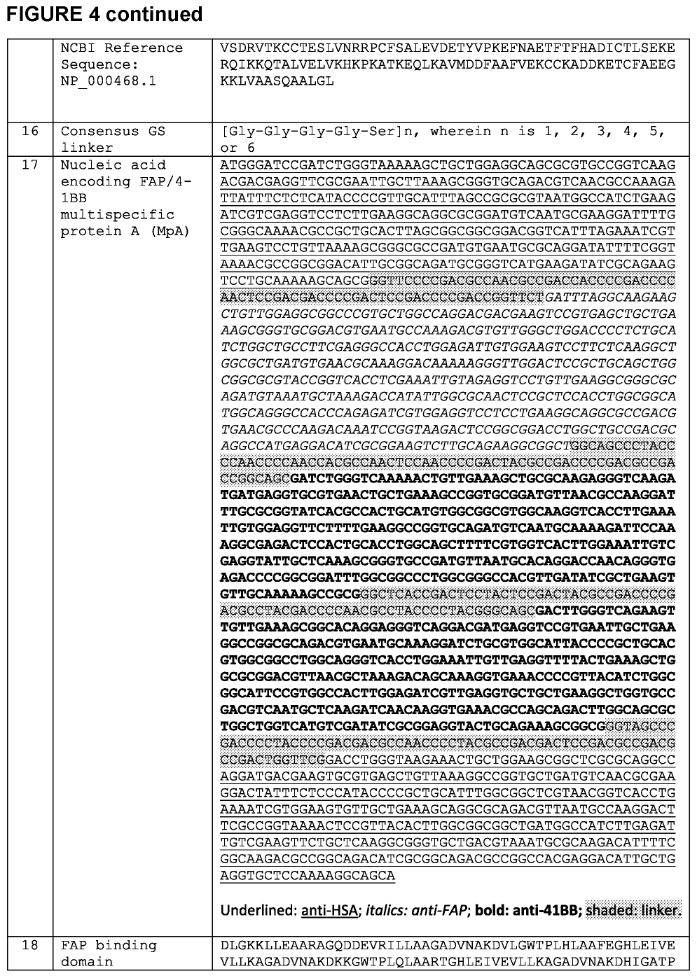

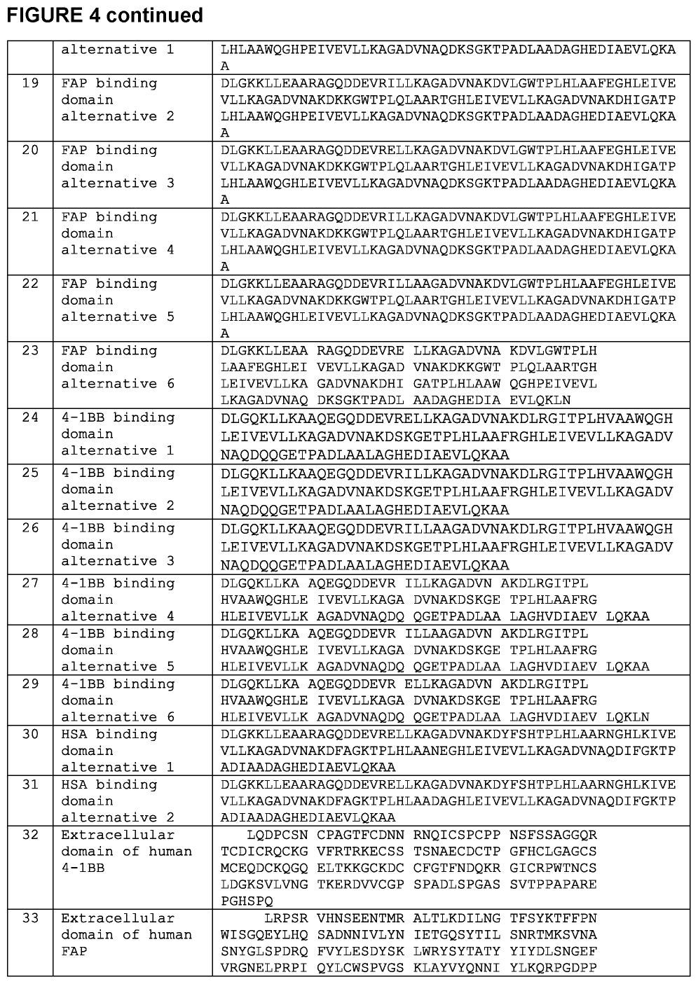

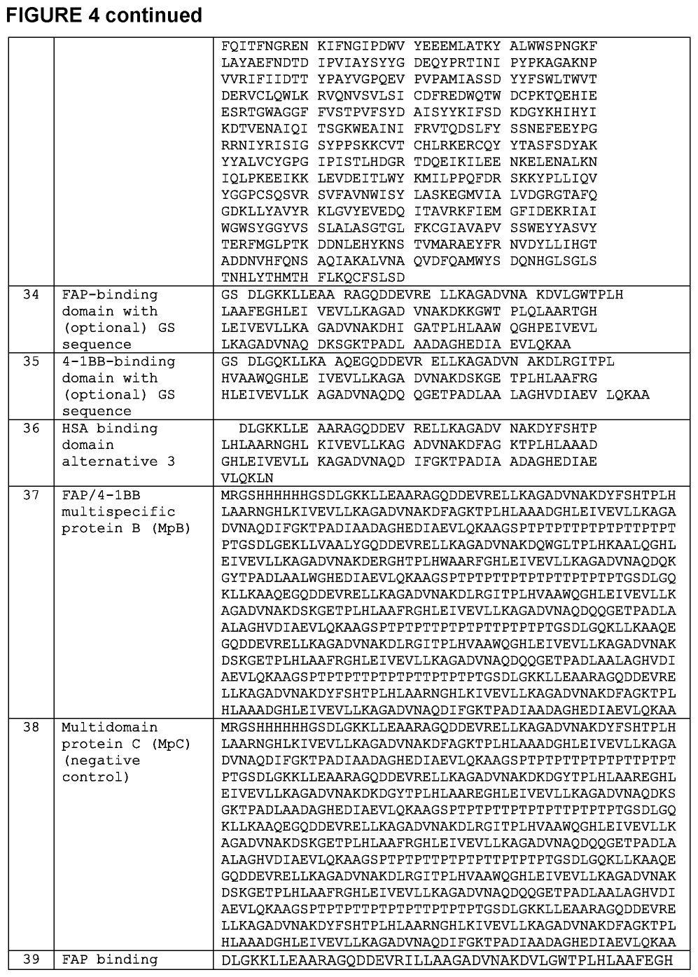

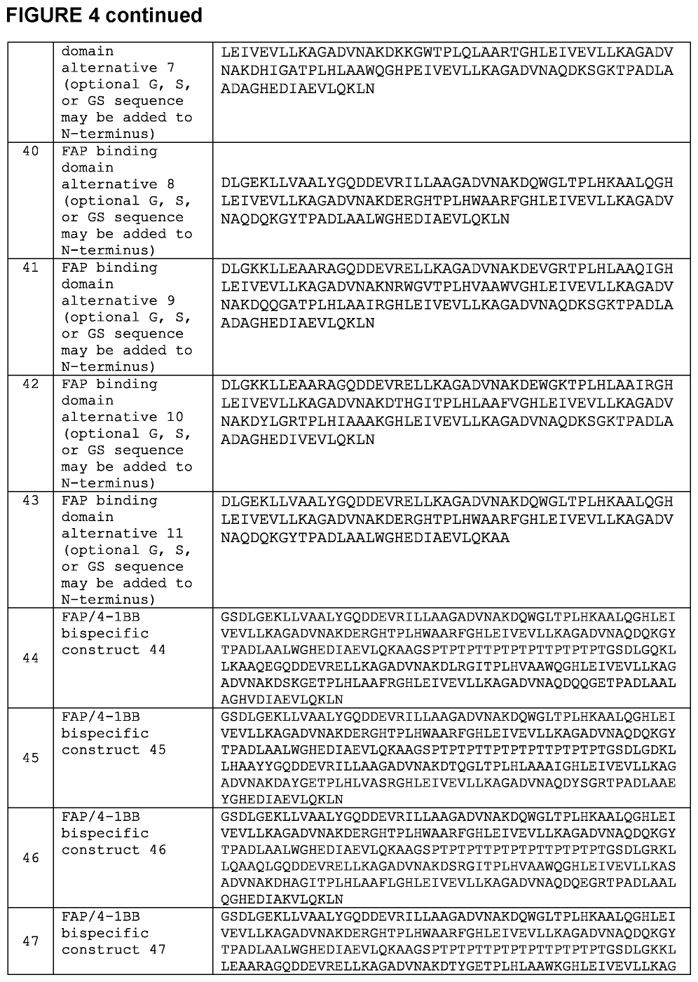

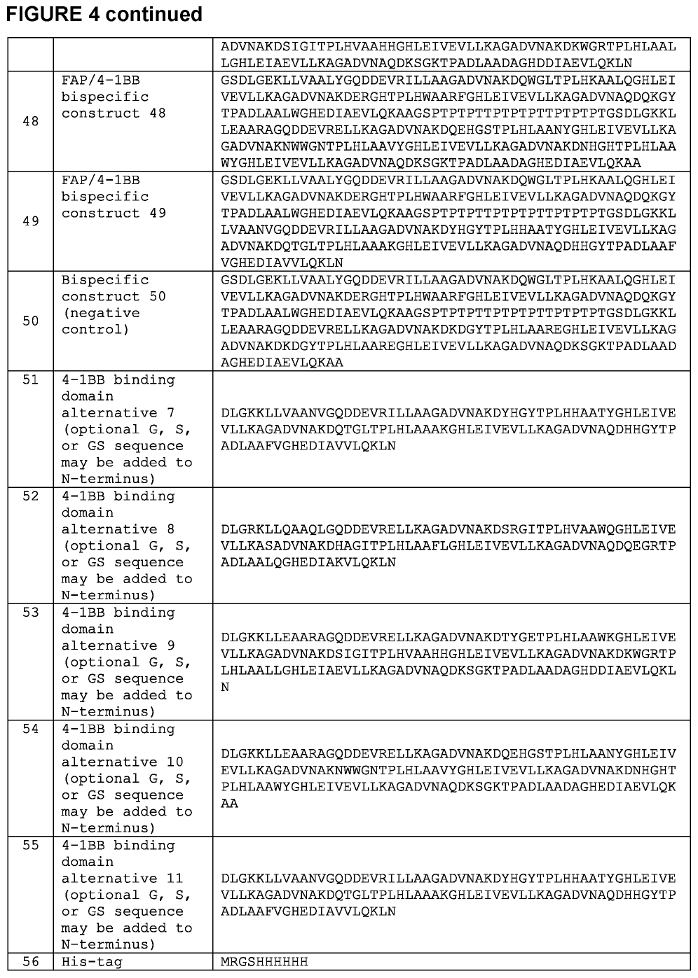

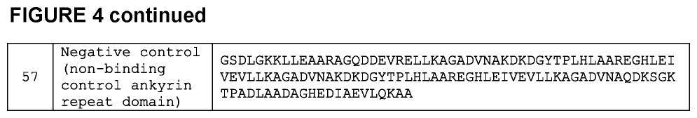

As shown in FIGS. 5A-5G, 4-1BB activation by the recombinant proteins of the invention depended on the presence of FAP-expressing cells (CHO-FAP). In the presence of cells that do not express FAP (CHO-wt), no activation of 4-1BB was observed. Although 4-1BB undergoes trimerization upon binding to its natural trimeric ligand (4-1BBL), having three 4-1BB binding domains is not required for 4-1BB activation in this case. Monovalent 4-1BB binder (F-B) was sufficient to activate 4-1BB. Higher potency was achieved by using two 4-1BB binding domains (F-B-B), or three 4-1BB binding domains (F-B-B-B). It was believed that by having three 4-1BB binding modules, the molecule would further promote 4-1BB clustering (“cross-linking” effect), thereby further enhancing T-cell mediated cytotoxicity. Surprisingly, as compared to F-B-B (two 4-1BB binding modules), the F-B-B-B format (three 4-1BB binding modules) did not significantly improve activity. Therefore, the F-B-B format was selected for further characterization. This format is used in Multispecific binding protein A (MpA) (SEQ ID NO: 6) described in the following examples.

### Example 2—Binding Affinity of Multispecific Molecules

The following Example describes experiments which were conducted to determine the species cross-reactivity of the different domains in Multispecific binding protein A (MpA), which comprises SEQ ID NO:6. The interaction of MpA with serum albumin, 4-1BB and FAP was analyzed for three species (human, cynomolgus monkey and mouse).

### Materials and Methods

ProteOn Setup for MpA Binding to 4-1BB of Different Species:

SPR measurements were performed using a ProteOn XPR36 instrument (BioRad). The running buffer was PBS pH 7.4 containing 0.005% Tween 20® (PBST). The bio.h4-1BB, bio.c4-1BB and bio.m4-1BB were immobilized on NLC chips (BioRad) to a level of 320 RU. The binding of MpA to 4-1BB was measured by injecting MpA in a serial dilution of 30, 10, 3.3, 1.1 and 0.3 nM with an association of 180 s and dissociation of 1800 s using a constant flow of 100 μl/min (see Table 3). The measurement was repeated three times and the targets were regenerated between the individual measurements using 10 mM glycine pH 2 and 124 mM H₃PO₄. The signals were double referenced against the running buffer (PBST) treated control lane of L1 and A6. h4-1BB, c-4-1BB, and m4-1BB refer to human, cynomolgus, and mouse orthologs of 4-1BB, respectively.

ProteOn Setup for MpA Binding to Serum Albumin of Different Species:

SPR measurements were performed using a ProteOn XPR36 instrument (BioRad). The running buffer was PBS pH 7.4 containing 0.005% Tween 20® (PBST). First, bio.h4-1BB was coated on NLC chip (BioRad) to a level of 320 RU before 100 mM MpA was immobilized for 180 s at constant flow of 30 μl/min to a level of 200 RU. The binding of the serum albumin to MpA was detected by applying HSA (human serum albumin), CSA (cynomolgus serum albumin) and MSA (mouse serum albumin) as titration with a 180 s association and 1800 s dissociation phase using a constant flow of 100 μl/min. HSA, CSA and MSA binding was measured serially and the bio.h4-1BB/MpA complex was regenerated each time with 10 mM glycine pH 2 and 124 mM H3PO4. Thus, MpA had to be recoated after each regeneration step. The signals were double referenced against the running buffer (PBST) treated control lane of L1 and A6. The 1:1 Langmuir model was used for the fitting.

ProteOn Setup for MpA Binding to FAP of Different Species:

SPR measurements were performed using a ProteOn XPR36 instrument (BioRad). The running buffer was PBS pH 7.4 containing 0.005% Tween 20® (PBST). hFAP (human FAP), cFAP (cynomolgus FAP) and mFAP (mouse FAP) were immobilized on a GLC chip (BioRad) at pH 5.3 to a level of 2000 RU, 1700 RU and 5000 RU, respectively. The interaction of FAP and MpA was measured by applying the MpA molecule as titration (see Table 2). The association and dissociation settings are summarized in Table 2.

TABLE 2

| | | Concentration | Flow | Association | Dissociation |
|---|---|---|---|---|---|
| Ligand | Analyte | Analyte [nM] | [μl/min] | [s] | [s] |
| Bio.h4-1BB* | MpA | 30, 10, 3.3, 1.1, 0.3 | 100 | 120 | 1800 |
| Bio.c4-1BB* | MpA | 30, 10, 3.3, 1.1, 0.3 | 100 | 120 | 1800 |
| Bio.m4-1BB* | MpA | 30, 10, 3.3, 1.1, 0.3 | 100 | 120 | 1800 |
| MpA | HSA | 50, 16.7, 5.6 1.9, 0.6 | 100 | 180 | 1800 |
| MpA | CSA | 100, 33, 11, 3.7, 1.2 | 100 | 180 | 1800 |
| MpA | MSA | 100, 33, 11, 3.7, 1.2 | 100 | 180 | 1800 |
| hFAP** | MpA | 25, 8.3, 2.8, 0.9, 0.3 | 100 | 120 | 1800 |
| hFAP** | MpA | 50, 25, 12.5, 6.25, 3.125 | 100 | 180 | 1500 |
| cFAP | MpA | 25, 8.3, 2.8, 0.9, 0.3 | 100 | 120 | 1800 |
| mFAP | MpA | 50, 25, 12.5, 6.25, 3.125 | 100 | 180 | 1500 |

*triplicate measurement using same settings
**duplicate measurement using different settings

Two independent measurements were conducted to detect the binding of MpA to hFAP. The targets were regenerated using 10 mM glycine pH 2 and 124 mM H3PO4. The signals were double referenced against the PBST treated control lane of L1 and A6. The 1:1 Langmuir model was used for the fitting.

### Results

Binding to 4-1BB:

Results showed that MpA binds to human 4-1BB with a K_{D}=13±5 pM and to cynomolgus 4-1BB with an K_{D}=14±8 pM (as shown in Table 3).

TABLE 3
Kinetic parameters of MpA binding to 4-1BB, serum albumin and FAP of different
species (human, mouse or cyno).

| Protein | Kₒₙ | K_{off} | K_{D} | STDEV K_{D} | Rmax | Chi²/Rmax^{†} |
|---|---|---|---|---|---|---|
| Name | [M⁻¹s⁻¹] | [s⁻¹] | [nM] | [nM] | [RU] | [%] |
| Bio.h4-1BB* | 2.6E+06 | 3.2E−05 | 0.013 | 0.005 | 155 | 17 |
| Bio.c4-1BB* | 2.1E+06 | 2.7E−05 | 0.014 | 0.008 | 140 | 19 |
| Bio.m4-1BB* | No specific binding detected | | | | | |
| HSA | 1.5E+06 | 1.2E−02 | 8 | n.d. | 241 | 36 |
| CSA | 1.1E+06 | 7.5E−02 | 67 | n.d. | 346 | 6 |
| MSA | 1.0E+06 | 3.8E−02 | 38 | n.d. | 251 | 29 |
| hFAP** | 4.7E+05 | 1.3E−04 | 0.4 | 0.2 | 221 | 18 |
| cFAP | 1.7E+05 | 1.3E−04 | 0.8 | n.d. | 230 | 3 |

| | |
|---|---|
| mFAP | No specific binding detected |

*The values represent the average of a triplicate measurement
**The values represent the average of a duplicate measurement
^{†}Chi²/Rmax > 10% defined as inaccurate fit

Thus, the results indicate that MpA binds to human and cyno 4-1BB with a similar apparent affinity. However, no binding to m4-1BB was detected indicating that MpA is not cross-reactive to mouse 4-1BB. The findings are in agreement with the sequence identities of 95% for human and cynomolgus and 56% for human and mouse.

Binding to Serum Albumin:

MpA was determined to be cross-reactive to human, cynomolgus and mouse serum albumin with affinities of K_{D}=8 nM, 67 nM and 38 nM, respectively. Thus, MpA has a 4-8 fold higher affinity for human serum albumin than to cynomolgus or mouse serum albumin. Different affinities to mouse and cyno might be due to individual mutations in epitope regions since the sequence identity of human versus cynomolgus or mouse is 93% and 72%, respectively. Although some degree of non-specific binding to the chip surface was observed for the highest applied concentrations of MSA and CSA (33-100 nM), the determined kinetic parameters for binding serum albumin fall into the expected range.

Binding to FAP:

MpA was determined to be cross-reactive to human and cynomolgus FAP but not to mFAP. MpA binds to hFAP with an affinity of K_{D}=0.4±0.2 nM and to cFAP with K_{D}=0.8 nM. The binding of MpA to human and cynomolgus FAP is characterized by a slow off-rate of 1.3E-04 s⁻¹. The similar affinity between human and cynomolgus FAP is in agreement with the sequence identity of 97%.

### Conclusion:

Surface plasmon resonance measurements have shown that MpA binds tightly to human and cynomolgus 4-1BB with apparent affinities of 13±5 pM and 14±8 pM, respectively. MpA is not cross-reactive to m4-1BB, potentially due to low sequence similarity of the extracellular domain of only 56%. MpA showed binding to human, cynomolgus and mouse serum albumin with affinities of 8 nM, 67 nM and 38 nM, respectively. Furthermore, it was shown that MpA binds to human and cynomolgus FAP in the subnanomolar range, while no cross-reactivity could be detected for mouse FAP despite a relatively high sequence similarity of 90%.

### Example 3—Simultaneous Binding of MpA to 4-1BB, FAP and Human Serum Albumin Analyzed by Surface Plasmon Resonance

The following experiment describes a surface plasmon resonance experiment that was performed to analyze the simultaneous binding of Multispecific binding protein A (MpA) comprising SEQ ID NO:6 to human 4-1BB, human FAP and human serum albumin, respectively.

The analysis was conducted with the following set-up: Before starting the binding measurement, biotinylated human 4-1BB was immobilized on a NeutrAvidin-coated NLC chip surface. In a first step, MpA was added which has a slow off rate from 4-1BB. Second, hFAP was applied as the second target followed by the addition of HSA as the third and final target.

SPR measurements were performed using a ProteOn XPR36 instrument (BioRad). PBS pH 7.4 containing 0.005% Tween 20 was used as running buffer. 360 RU of 10 nM biotinylated human 4-1BB (bio.h4-1BB-Fc) was immobilized on a NeutrAvidin coated NLC sensor chip. To the bio.4-1BB immobilized chip successively 100 nM MpA, association 180 s, dissociation 60 s, 100 nM hFAP association 180 s, dissociation 60 s and 100 nM human serum albumin association 180 s, dissociation 1000 s were applied as independent analyte steps. The setup allows binding of hFAP and HSA only if MpA is already bound to 4-1BB. A requirement for this set-up was that MpA binds 4-1BB and FAP with high affinity to prevent rapid signal loss before applying the third target (HSA). Different analyte lanes were used to include all controls. The signals were double referenced to the PBST treated control lane of L1 and A6. Additionally, the analysis of the amounts (RUs) of immobilized ligands allowed the determination of the binding stoichiometry of the complex by using the following formula: Valency_{ligand}=(Rₘₐₓ×MW_{ligand})/(R_{ligand}*MW_{analyte}).

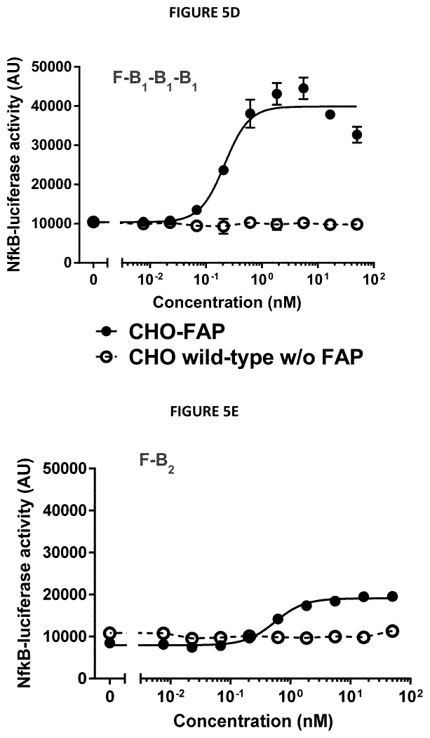

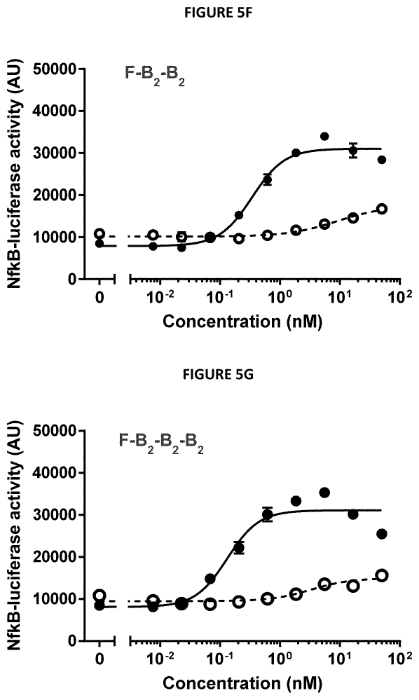

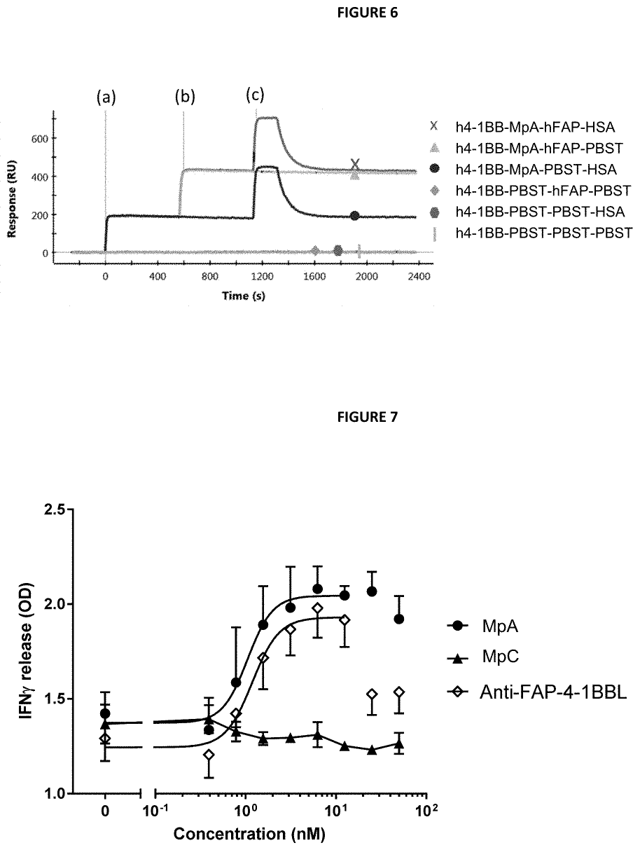

Initially, before starting the binding measurement, around 360 response units (RUs) of human 4-1BB were immobilized on the SPR chip. In a first step, 200 RU of MpA were bound to the immobilized h4-1BB (shown in FIG. 6 as injection (a)). Second, hFAP was injected and binding to MpA could be shown (indicated by an increase of 220 RUs) (shown in FIG. 6 as injection (b)). Third, HSA was injected (shown in FIG. 6 as injection (c)). The binding of human serum albumin in the subsequent association phase indicates that simultaneous binding of MpA to all three targets is possible. In addition, the analysis of the maximal amounts of response units after each injection step allowed the quantification of the binding valency (summarized in Table 4).

TABLE 4
Response units (RU) and binding stoichiometry of each binding
event during simultaneous binding SPR measurement.

| Step | Analyte (A) | | Ligand (L) | | Binding |
|---|---|---|---|---|---|
| (Injection) | Name | Rₘₐₓ | Name | R_{L} | stoichiometry |
| 1 | MpA | 200 | bio.41BB-Fc | 360 | 0.7 |
| 2 | hFAP | 220 | Complex 1* | 560 | 0.8 |
| 3 | HSA | 280 | Complex 2** | 780 | 1.4 |

*Complex 1: bio.4-1BB-Fc/MpA
**Complex 2: bio.4-1BB-Fc/MpA/hFAP

Thus, an RU of around 280 upon binding of HSA implies that two HSA molecules can bind to MpA simultaneously. By analogy, the binding ratio of MpA to 4-1BB and FAP was determined as 1:1. Together with the observed high apparent binding affinity due to the bivalency of MpA against h4-1BB (low off-rate), it was determined that MpA was able to bind two immobilized h4-1BB molecules.

### Example 4—Costimulation of Human T Cell Activation Via 4-1BB

The aim of the study was to determine the potency of multispecific binding protein A (MpA), comprising SEQ ID NO:6, to co-stimulate activation of primary human CD8 T cells and enhance the anti-CD3-mediated IFNγ production by primary human CD8 T cells in vitro. The functional activity of MpA to enhance anti-CD3-mediated IFNγ production by primary human CD8 T cells was compared to several other 4-1BB agonistic molecules. MpA was able to enhance IFNγ secretion by CD8 T cells in a dose-dependent manner with an EC₅₀ of 1-2 nM, in the presence of plate-coated FAP. Therefore, it was determined that MpA is able to provide potent co-stimulation to primary human CD8 T cells when it is bound to FAP. The potency of MpA was comparable to anti-FAP-4-1BBL (EC₅₀ 1-2 nM), the fusion of the natural trimeric ligand of 4-1BB to an anti-FAP antibody.

### Materials and Methods:

In Vitro Human T Cell IFNγ Release Assay Using FAP-Clustering:

Buffy coats were obtained from the Zurich blood donation centre and diluted with PBS. PBMCs were then isolated by density centrifugation using Leucosep tubes. After several washing steps, CD8 T cells were purified from PBMCs using a negative selection human CD8 T cell isolation Kit according to the manufacturer's recommendations. CD8 T cells (1×10⁵/well) were seeded onto 96-well plates previously coated with 0.5 μg/ml anti-CD3 clone OKT-3 and Neutravadin followed by biotinylated hFAP in the presence of varying concentrations of test items. Cultures were incubated for 96 hours at 37° C., 5% CO₂ after which the supernatant was removed into a fresh 96-well plate and stored at −20° C. until analysis. The IFNγ concentration of the supernatant was detected using the human IFN-gamma DuoSet ELISA according to the manufacturer's instructions. EC₅₀ values were determined by fitting the data with the four-parameter logistical fit model using Graphpad Prism software.

In Vitro Human T Cell IFNγ Release Assay Using Anti-Fc-Clustering:

Buffy coats were obtained from the Zurich blood donation centre and diluted with PBS. PBMCs were isolated by density centrifugation using Leucosep tubes. After several washing steps, CD8 T cells were purified from PBMCs using a negative selection human CD8 T cell isolation Kit according to the manufacturer's recommendations. CD8 T cells (1×10⁵/well) were seeded onto 96-well plates previously coated with 1 μg/ml anti-CD3 (clone OKT-3) and varying concentrations of anti-4-1BB antibody, also coated to the well via anti-human IgG. Cultures were incubated for 96 h at 37° C., 5% CO₂ after which the supernatant was removed into a fresh 96-well plate and stored at −20° C. until analysed. The IFNγ concentration of the supernatant is detected using the human IFN-gamma DuoSet ELISA according to the manufacturer's instructions.

EC₅₀ Determinations:

EC₅₀ values were determined using GraphPad Prism version 7.02 by converting the x values (concentrations) in a log mode and fitting in a non-linear mode log (agonist) vs. response with a variable slope (four parameter) equation for determination of EC₅₀ values.

Negative Control:

Multidomain protein C (MpC) comprising SEQ ID NO:38 is used as negative control, to demonstrate dependence of the pharmacological activity of MpA on binding to FAP. Similar to MpA, MpC comprises five ankyrin repeat domains: HSA-non-FAP-4-1BB-4-1BB-HSA. The HSA and 4-1BB binding domains are the same as in MpA, but the “non-FAP” domain is a control ankyrin repeat domain that does not have a binding target. A hexa-histidine tag was added to facilitate detection.

Mouse Surrogate:

Multispecific binding protein B (MpB) comprising SEQ ID NO:37 is a mouse surrogate comprising five ankyrin repeat domains: HSA-FAP*-4-1BB-4-1BB-HSA. The HSA and 4-1BB binding domains are the same as in MpA. FAP* is an ankyrin repeat domain that specifically binds mouse FAP and human FAP. MpB was used to demonstrate pharmacological activity in humanized mouse models. A hexa-histidine tag was added to facilitate detection.

Anti-FAP-4-1BBL:

Anti-FAP-4-1BBL is a fusion protein comprising natural human 4-1BB ligand (4-1BBL) fused to an anti-FAP antibody (WO 2016/075278).

### Results and Discussion:

MpA Enhances IFNy Secretion of Ex Vivo Human CD8 T Cells:

MpA was assessed for its ability to activate 4-1BB on human CD8 T cells when presented to the cells via cross-linking to FAP bound to the plate. MpA induced IFNγ secretion of CD8 cells in a concentration-dependent manner with an EC₅₀ of 1-2 nM (FIG. 7). Conversely, negative control MpC, which contains the same 4-1BB binding domains as MpA but no FAP binding domain, did not result in stimulation of the CD8 cells, suggesting that clustering of MpA via FAP is essential for activation of the CD8 T cells. The anti-FAP-4-1BBL molecule containing the natural human 4-1BB ligand fused to an anti-FAP antibody showed similar potency in this assay with an EC₅₀ of 1-2 nM (FIG. 7). MpB, a mouse surrogate of MpA, comprising a FAP binding domain that binds mouse and human FAP and the same 4-1BB binding domains as MpA, was also able to activate primary human CD8 T cells with comparable EC₅₀ of 1-2 nM. MpA-His had a similar EC₅₀ of 1-3 nM. EC₅₀ values from representative experiments are summarized in Table 5.

TABLE 5
Potency of FAP-targeted agonists in
human CD8 T Cell Activation assay.

| Protein/Inhibitor | EC₅₀ [nM] | 95% Cl |
|---|---|---|
| MpA | 1.17 | 0.67 to 3.44 |
| MpC | No activation | — |
| Anti-FAP-4-1BBL | 1.26 | 0.70 to 2.89 |
| His-MpA | 2.37 | 1.63 to 3.45 |
| (His-tagged version of MpA) | | |
| MpB | 1.54 | 0.77 to 3.19 |

Co-Stimulatory Activity of Anti-4-1BB Antibodies Depends on Fc-Cross-Linking:

Anti-4-1BB mAbs enhance the anti-CD3-mediated activation of isolated human primary CD8 T cells (Fisher et al., Cancer Immunol Immunother 61, 1721-1733 (2012). The agonistic activity of anti-4-1BB mAbs has been shown to depend on clustering of the bound antibodies via their Fc-receptors or by coating on plate surfaces. The potency of the anti-4-1BB mAbs 20H4.9 (IgG4; U.S. Pat. No. 7,288,638) and MOR-7480 (WO 2012/032433) to enhance anti-CD3-mediated IFNγ production by isolated human primary CD8 T cells in soluble form or bound to plate-coated anti-Fc antibody were compared. In this assay, anti-4-1BB mAb 20H4.9 was able to enhance IFNγ production without Fc-cross-linking with an EC₅₀ of 0.97 nM. Cross-linking via coated anti-Fc increased the potency by approximately 25-fold to an EC₅₀ of 0.04 nM. Anti-4-1BB mAb MOR-7480, on the other hand, did not show any agonist activity in soluble form at any concentration tested. Fc-mediated cross-linking of anti-4-1BB mAb MOR-7480 resulted in enhanced IFNγ production with an EC₅₀ of 0.42 nM. A comparison of potencies of anti-4-1BB mAbs 20H4.9 and MOR-7480 with cross-linking via anti-Fc antibodies showed a roughly 10-fold superior potency of anti-4-1BB mAb 20H4.9 in this assay. Anti-FAP-4-1BBL showed potency with an EC₅₀ of 0.11 nM when cross-linked via plate-coated anti-Fc. EC₅₀ values are summarized in Table 6.

TABLE 6
Potency of anti-4-1BB antibodies in
human CD8 T Cell Activation assay.

| Protein/Inhibitor | EC₅₀ [nM] | 95% Cl |
|---|---|---|
| Anti-4-1BB mAb 20H4.9 | 0.02 | 0.007 to 0.04 |
| cross-linked via anti-Fc | | |
| Anti-4-1BB mAb 20H4.9 soluble | 0.93 | 0.71 to 1.22 |
| Anti-4-1BB mAb MOR-7480 | 0.21 | 0.13 to 0.34 |
| cross-linked via anti-Fc | | |
| Anti-4-1BB mAb MOR-7480 soluble | No activation | — |
| Anti-FAP-4-1BBL | 0.04 | 0.009 to 0.15 |

### Conclusion:

The aim of the study was to determine the potency of MpA to co-stimulate activation of CD8 T cells and enhance the anti-CD3-mediated IFNγ production by primary human CD8 T cells in vitro and to compare it to the potencies of the anti-4-1BB monoclonal antibodies 20H4.9 and MOR-7480. The functional activity of MpA to enhance anti-CD3-mediated IFNγ production by primary human CD8 T cells was compared to several other 4-1BB agonistic molecules. MpA was able to enhance IFNγ secretion by CD8 T cells in a dose-dependent manner with an EC₅₀ of 1-2 nM, in the presence of plate-coated FAP. MpC, the non-FAP-targeted control, showed no enhancement of IFNγ production. Therefore, it was determined that MpA is able to provide potent co-stimulation to primary human CD8 T cells when it is bound to FAP. The potency of MpA was comparable to anti-FAP-4-1BBL (EC50 1-2 nM), the fusion of the natural trimeric ligand of 4-1BB to an anti-FAP antibody.

The functional activity of anti-4-1BB mAbs 20H4.9 and MOR-7480 to enhance anti-CD3-mediated IFNγ production by CD8 T cells was assessed in a variation of the assay format using plates coated with anti-Fc antibody instead of FAP. Both antibodies required cross-linking via the anti-Fc antibody for full activity but several differences in the potencies of the antibodies were noted. Anti-4-1BB mAb 20H4.9 showed some activity without cross-linking (EC₅₀ 0.97 nM) whereas anti-4-1BB mAb MOR-7480 was inactive without cross-linking over the whole concentration range tested. In the presence of coated anti-Fc, anti-4-1BB mAb MOR-7480 was able to enhance CD8 T cell activation (EC₅₀ 0.42 nM) but anti-4-1BB mAb 20H4.9 showed approximately 10-fold higher potency (EC₅₀ 0.04 nM). Anti-4-1BBL was also active in this Fc cross-linking assay format with an EC₅₀ of 0.11 nM compared to an EC₅₀ of 1-2 nM in the FAP-dependent assay setup. The assay format therefore influences the overall potency of the molecules and consequently EC₅₀ values of different molecules assessed with the FAP-dependent or anti-Fc dependent assay formats should not be compared directly. Therefore, lower EC₅₀ values of the antibodies tested in the assay utilizing Fc-specific anti-IgGs do not necessarily mean that they have better agonistic potency as compared to FAP-targeted 4-1BB-specific reagents.

### Example 5—Pharmacokinetics of Multispecific Binding Protein a (MpA) in the Mouse

The objective of the pharmacokinetic (PK) study was to assess the PK characteristics of Multispecific binding protein A (MpA) comprising SEQ ID NO:6 in mice following single intravenous administration at a target dose level of 1 mg/kg.

Following single intravenous bolus injection, the concentration-time profile indicates a fast initial decline of the serum concentration which lasted up to approximately 6 h post compound administration followed by a slow decline which resembled a mono-exponential decay between 6 h and 168 h (the last time point analyzed). An apparent mean terminal half-life of 44.8 h was determined. The inter-subject variability of the serum concentrations measured at the same time points was low (<factor 2).

Using non compartmental analysis, exposure (AUCinf), total body clearance (CI), and volume of distribution were calculated to be: AUCinf=15600 h*(nmol/L), CI=0.826 mL/(h*kg), and Vss=51.5 mL/kg. The value determined for volume of distribution indicates that MpA is largely confined to the systemic circulation of the animals.

### Materials/Methods:

In Vivo Experiment:

Healthy female BALB/c mice (body weight 20.6-23.3 g prior to dosing) were supplied by Janvier, Saint Berthevin Cedex, France. During the pretrial period as well as during the trial, the animals were housed in groups in caging appropriate to the species. Animals had free access to a standard laboratory diet of known formulation (No. 3437, Provimi Kliba, Kaiseraugust, Switzerland) and domestic mains quality water was available ad libitum. Animals were individually marked before the start of the trial.

MpA was administered as a single intravenous bolus injection into the tail vein of each of 6 mice. The target dose level was 1 mg/kg with an application volume of 5 mL/kg. MpA was formulated in phosphate-buffered saline (PBS) solution (Gibco Life Technologies, Grand Island, N.Y., USA, Ref.: 10010-015).

Mice were split into two groups with equal numbers of animals. Four serum samples were collected from each mouse. Blood samples for pharmacokinetic investigations (approximately 50 μI/sample) were collected from the saphenous vein at 5 min, 6 h, 24 h, 48 h, 72 h, 96 h and 168 h post compound administration into Multivette 600 tubes. The assignment of the individual animals to the respective sampling time points is given in the serum concentration-time data table (Table XXX below). Blood was kept at room temperature for approximately 30 minutes to allow clotting followed by centrifugation (5 min/12000 g/4° C.). Serum was frozen and stored at −20° C. pending analyses.

Bioanalystics (ELISA):

One hundred μl per well of 1.9 nmol/L rabbit monoclonal anti-DARPin® antibody 1-1-1 in PBS was coated onto a NUNC Maxisorb ELISA plate overnight at 4° C. After washing with 300 μl PBST (PBS supplemented with 0.1% Tween20) per well five times, the wells were blocked with 200 μl PBST supplemented with 0.25% Casein (PBST-C) for 1 h at room temperature (RT) on a Heidolph Titramax 1000 shaker (450 rpm). Plates were washed as described above. One hundred μl of diluted serum samples (1:20-1:312500 in 1:5 dilution steps) or MpA standard curve samples (0 and 50-0.0008 nmol/L in 1:3 dilution steps) were applied for 2 h, at RT, shaking at 450 rpm. Plates were washed as described above. Wells were then incubated with 100 μl human anti-DARPin® monoclonal Ab 1.4.8 (500 ng/mL) in PBST-C for 1 h, RT, 450 rpm. Plates were washed as described above. Wells were then incubated with 100 μl goat anti-human IgG/HRP conjugate (Ab15, 500 ng/mL in PBST-C) and incubated for 1 h, at RT, 450 rpm. Plates were washed as described above. The ELISA was developed using 50 μl/well TMB substrate solution for 5 min and stopped by the addition of 50 μl 1 mol/L H2504. The difference between the absorbance at 450 nm and the absorbance at 620 nm was calculated. Samples were measured in duplicate on two different plates. Absorbance values of diluted serum samples were compared to the standard curve to calculate serum concentrations of the samples. LLOQ of the assay was 1 nmol/L.

Pharmacokinetic Analysis:

The following pharmacokinetic parameters were calculated: AUCinf, AUClast, AUC_%extrapol, Cmax, Tmax, CI_pred, Vss_pred, t1/2.

Maximum serum concentrations (Cmax) and the times of their occurrence (Tmax) were obtained directly from the serum concentration-time profiles. The area under the serum concentration-time curve (AUCinf) was determined by the linear trapezoidal formula up to the last sampling point (Tlast) and extrapolation to infinity assuming mono-exponential decrease of the terminal phase. The extrapolation up to infinity was performed using Clast/λz, where λz denotes the terminal rate constant estimated by log linear regression and Clast denotes the concentration estimated at Tlast by means of the terminal log-linear regression. Serum concentration-time points used for this extrapolation are marked with (*) in the serum concentration-time data table (Table 7 below). Total serum clearance (CI_pred) and the apparent terminal half-life were calculated as follows: CI_pred=i.v. dose/AUCinf and t1/2=ln 2/λz. The steady-state volume of distribution Vss was determined by: Vss=i.v. dose·AUMCinf/(AUCinf)². AUMCinf denotes the total area under the first moment of drug concentration-time curve extrapolated to infinity using the same extrapolation procedure as described for calculation of AUCinf.

To calculate PK parameters based on concentrations given in nmol/L dose values given as mg/kg were converted to nmol/kg by using the molecular weight of MpA of 77713 g/mol. Thereby the dose level of 1 mg/kg was converted to 12.87 nmol/kg.

### Results and Discussion:

In Vivo Animal Experiment:

MpA was administered to female BALB/c mice as a single intravenous bolus injection into the tail vein. The target dose level in the study was 1 mg/kg.

For each study, 6 mice were split into 2 groups with equal numbers of animals. For pharmacokinetic investigations, 4 serum samples from each mouse were collected from the saphenous vein at various time points. The assignment of the individual animals to the respective sampling time points is given in the serum concentration-time data table (Table 7 below). Serum was frozen at 20° C. pending analyses.

No major issues and no drug-related adverse effects were reported for the in vivo experiment.

Bioanalystics (ELISA):

Serum concentrations of MpA were determined by sandwich ELISA using plate-bound rabbit monoclonal anti-DARPin® IgG 1-1-1 to capture MpA in diluted serum samples. Human anti-DARPin® antibody 1.4.8 followed by a conjugate of goat anti-human IgG and horseradish peroxidase (HRP) was used for detection. The MpA concentration in each serum sample was determined using a standard curve.

Individual and mean serum concentration-time data are summarized in Table 7 below. The inter-subject variability of the serum concentrations measured at the same time points was low (<factor 2).

Pharmacokinetic Analysis:

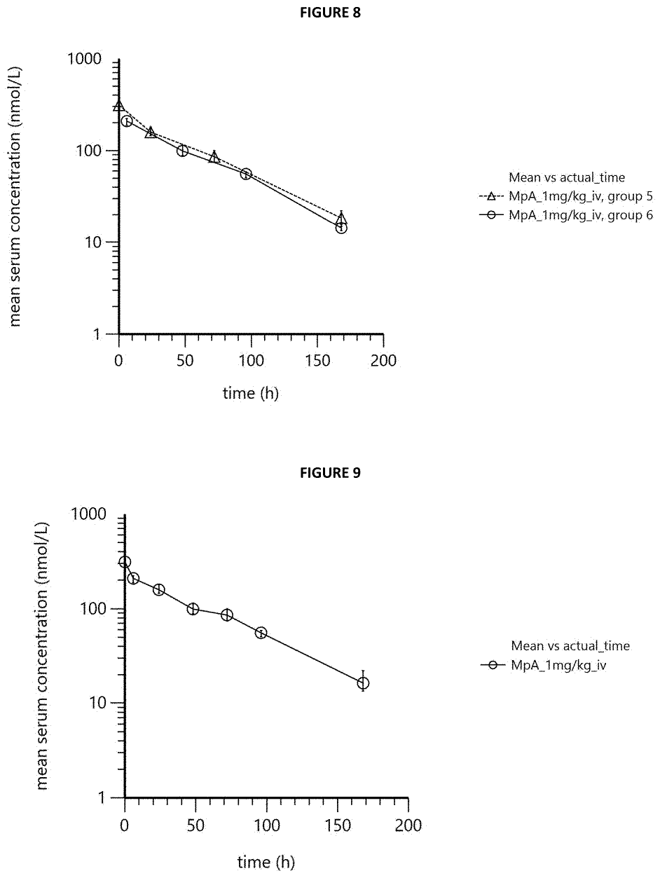

Individual serum concentration-time data of MpA in BALB/c mice following single intravenous administration of 1 mg/kg are shown in Table 7. The corresponding profiles showing the group mean (+/− max/min) or overall mean (+/− max/min) of the serum concentrations are given in FIG. 8 and FIG. 9, respectively.

Non-compartmental analysis (NCA) was performed using the mean concentration-time data. Selected data points for determination of half-life are given in Table 7 (indicated by asterisks).

TABLE 7
Individual and mean serum concentrations of MpA in BALB/c mice following single
intravenous administration of 1 mg/kg

| | | | Serum | Mean Serum | | | |
|---|---|---|---|---|---|---|---|
| | Group | Animal | Concentration | Concentration | SD | Min | Max |
| Time (h) | No/cage | No | (nmol/L) | (nmol/L) | (nnol/L) | (nmol/L) | (nmol/L) |
| 0.08333 | group 5 | 1 | 336 | 312 | 24.7 | 287 | 336 |
| | EMMA- | 2 | 312 | | | | |
| | 00198 | 3 | 287 | | | | |
| 6 | group 6 | 4 | 218 | 209* | 18.5 | 188 | 221 |
| | EMMA- | 5 | 188 | | | | |
| | 00199 | 6 | 221 | | | | |
| 24 | group 5 | 1 | 180 | 158* | 18.4 | 145 | 180 |
| | EMMA- | 2 | 145 | | | | |
| | 00198 | 3 | 150 | | | | |
| 48 | group 6 | 4 | 115 | 99* | 14.0 | 88.3 | 115 |
| | EMMA- | 5 | 88.3 | | | | |
| | 00199 | 6 | 94.1 | | | | |
| 72 | group 5 | 1 | 99.4 | 86* | 12.8 | 74.0 | 99.4 |
| | EMMA- | 2 | 74.0 | | | | |
| | 00198 | 3 | 83.9 | | | | |
| 96 | group 6 | 4 | 59.1 | 56* | 4.81 | 50.1 | 59.1 |
| | EMMA- | 5 | 57.7 | | | | |
| | 00199 | 6 | 50.1 | | | | |
| 168 | group 5 | 1 | 22.3 | 16.4* | 3.37 | 13.3 | 22.3 |
| | EMMA- | 2 | 14.8 | | | | |
| | 00198 | 3 | 18.3 | | | | |
| | group 6 | 4 | 15.6 | | | | |
| | EMMA- | 5 | 13.9 | | | | |
| | 00199 | 6 | 13.3 | | | | |

*Values used for calculation of terminal half-life

Following single intravenous bolus injection of 1 mg/kg MpA, the concentration-time profile indicates a fast initial decline of the serum concentration which lasted up to approximately 6 h post compound administration followed by a slow decline resembling a mono-exponential decay between 6 h and 168 h (the last time point analyzed). An apparent mean terminal half-life of 44.8 h was determined.

Using non-compartmental analysis, exposure (AUCinf), total body clearance (CI), and volume of distribution were calculated to be: AUCinf=15600 h*(nmol/L), CI=0.826 mL/(h*kg), and Vss=51.5 mL/kg. The value determined for volume of distribution indicates that MpA is largely confined to the systemic circulation of the animals.

Conclusion:

The pharmacokinetic analysis indicates that MpA has an apparent terminal half-life of 44.8 h in mice following administration of 1 mg/kg of the test compound by intravenous bolus injection. Using non-compartmental analysis, exposure (AUCinf), total body clearance (CI), and volume of distribution were calculated to be: 15600 h*(nmol/L), 0.826 mL/(h*kg) and 51.5 mL/kg. The value determined for volume of distribution indicates that MpA is largely confined to the systemic circulation.

### Example 6—Pharmacokinetic Analysis of MpA in Cynomologous Monkeys

The objective was to assess the PK characteristics of MpA comprising an additional N-terminal hexa-histidine tag (His-MpA) in protein-naïve cynomolgus monkey following single doses of 0.1, 1 and 10 mg/kg given as an intravenous infusion over 30 min. For the study, one monkey (Macaca fascicularis) was used for each dose level. Serum samples were taken over a 13 day period. Serum concentrations of the His-MpA were measured by sandwich ELISA. Measurements of anti-His-MpA antibodies (AMA) were measured by ELISA.

### Material/Methods:

In vivo animal experiment: Female animals were dosed with His-MpA in PBS+0.05% Tween 20 formulation via intravenous infusion over 30 min at target dose levels of 0.1, 1 and 10 mg/kg and in an administration volume of 5.0 mL/kg. Samples for pharmacokinetic evaluation were collected pre-dose and again at nominal time points of 10 min and 3, 8, 24, 48, 72, 96, 120, 144, 192, 240, 288 and 312 hours post-end of infusion. Pre-dose sample were not measured for His-MpA concentrations. Samples for AMA determination were collected prior to administration (day −4) and again at nominal time points of 96, 120, 144, 192, 240, 288 and 312 hours post-end of infusion. Samples taken at 120 and 288 h were not measured for anti-drug antibodies (ADA). Nominal sampling time points were actually met for the samples up to 48 h and slightly varied afterwards (72+/−0.25 h, 96-288+/−0.75 h, 312+/−1 h). Nominal time points were used for the evaluation of concentration-time and AMA-time data evaluation.

ELISA to Measure his-MpA in Serum Samples:

One hundred μl of 10 nM polyclonal goat anti-rabbit IgG antibody (Ab18) in PBS was coated onto a NUNC Maxisorb ELISA plate overnight at 4° C. After washing with 300 μl PBST (PBS supplemented with 0.1% Tween20) per well five times, the wells were blocked with 200 μl PBST supplemented with 0.25% Casein (PBST-C) for 1 h at room temperature (RT) on a Heidolph Titramax 1000 shaker (450 rpm). Plates were washed as described above. 100 μl 5 nM rabbit anti-DARPin® 1-1-1 antibody in PBST-C was added and the plates were incubated at RT (22° C.) with orbital shaking (450 rpm) for 1 h. Plates were washed as described above.

100 μl of diluted serum samples (1:20-1:62500 in 1:5 dilution steps) or His-MpA standard curve samples (0 and 50-0.0008 nmol/L in 1:3 dilution steps) were applied for 2 h at RT, shaking at 450 rpm. Plates were washed as described above.

Wells were then incubated with 100 μl murine anti-RGS-His-HRP IgG (Ab06, 1:2000 in PBST-C) and incubated for 1 h at RT, 450 rpm. Plates were washed as described above. The ELISA was developed using 50 μl/well TMB substrate solution for 5 minutes and stopped by the addition of 50 μl 1M H2504. The difference between the absorbance at 450 nm and the absorbance at 620 nm was calculated. Samples were measured in duplicate on two different plates. LLOQ of the assay was 1 nmol/L.

ELISA to Measure Anti-his-MpA Antibodies in Serum Samples:

One hundred μl of 1 μg/mL His-MpA in PBS were coated onto a NUNC Maxisorb ELISA plate overnight at 4° C. After washing with 300 μl PBST (PBS supplemented with 0.1% Tween20) per well five times, the wells were blocked with 300 μl PBST supplemented with 0.25% Casein (PBST-C) for 1 h at room temperature (RT) on a Heidolph Titramax 1000 shaker (450 rpm). Plates were washed as described above. 100 μl of diluted serum samples (starting dilution 1:100 (=minimum required dilution, MRD)-1:4882812500 in 1:5 dilution steps) or anti-DARPin® antibody positive control (diluted the same way as the samples) was applied. For cut-point determination, 12 sera of naïve cynomolgus monkeys which were pre-tested to result in low signals were applied (100 μl) on the same plate at the MRD of 1:100. Plates were incubated for 2 h, at RT, shaking at 450 rpm. Plates were washed as described above. Wells were then incubated with 100 μl goat-anti-hIgG-HRP (Ab15, 0.5 μg/mL in PBST-C) and incubated for 45 min at RT, 450 rpm. Ab15 is cross-reactive to monkey IgG and recognizes IgG of cynomolgus monkeys. Plates were washed as described above. The ELISA was developed using 50 μl/well TMB substrate solution for 5 minutes and stopped by the addition of 50 μl 1M H2504. The difference between the absorbance at 450 nm and the absorbance at 620 nm was calculated. Samples were measured in duplicate on two different plates.

Cut-point values were calculated for each plate from the optical density of the twelve cut-point sera by multiplying the standard deviation of these twelve cut-point sera with the normal distribution factor 2.576 (99% of all values are within the normal distribution) plus the mean value of these twelve cut-point sera.

The AMA titer of the serum samples were calculated from the intersection point of the serum titration curve and the cut-point value using a 4-parameter fitting algorithm. Serum samples which resulted in an optical density below the cut-point value at MRD were considered AMA-negative.

Pharmacokinetic Analysis:

Pharmacokinetic data analysis was performed using Version 7.0 of the WinNonlin program as part of Phoenix 64, Pharsight, North Carolina. Calculation of the pharmacokinetic parameters based on the concentration-time data of the animals dosed via intravenous infusion was performed with non-compartmental analysis (NCA model 200-202, IV infusion, linear trapezoidal linear interpolation, infusion time was set to 0.5 h lasting from time point minus 0.5 h until time point 0 h). The following pharmacokinetic parameters were calculated:
AUCinf, AUClast, AUC_%extrapol, Cmax, Tmax, CI_pred, Vss_pred, t1/2

The area under the serum concentration-time curve (AUCinf) was determined by the linear trapezoidal formula up to the last sampling point (Tlast) and extrapolation to infinity assuming mono-exponential decrease of the terminal phase. The extrapolation up to infinity was performed using Clast/λz, where λz denotes the terminal rate constant estimated by log linear regression and Clast denotes the concentration estimated at Tlast by means of the terminal log-linear regression. Serum concentration-time points used for this extrapolation are marked with (*) in the serum concentration-time data tables and are provided in FIGS. 13 to 17. Total serum clearance (CI_pred) and the apparent terminal half-life were calculated as follows: CI_pred=i.v. dose/AUCinf and t1/2=ln 2/λz. The steady-state volume of distribution Vss was determined by: Vss=i.v. dose·AUMCinf/(AUCinf)². AUMCinf denotes the total area under the first moment of drug concentration-time curve extrapolated to infinity using the same extrapolation procedure as described for calculation of AUCinf.

To calculate PK parameters based on concentrations given in nmol/L, dose values given as mg/kg were converted to nmol/kg by using the molecular weight of His-MpA of 78968 g/mol. Dose-normalization of exposure data was performed by using the dose given in mg/kg.

### Discussion:

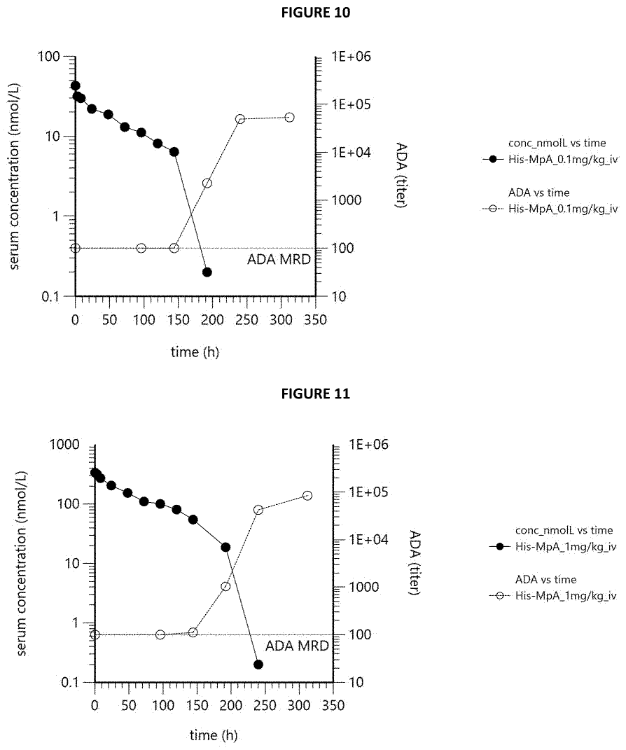

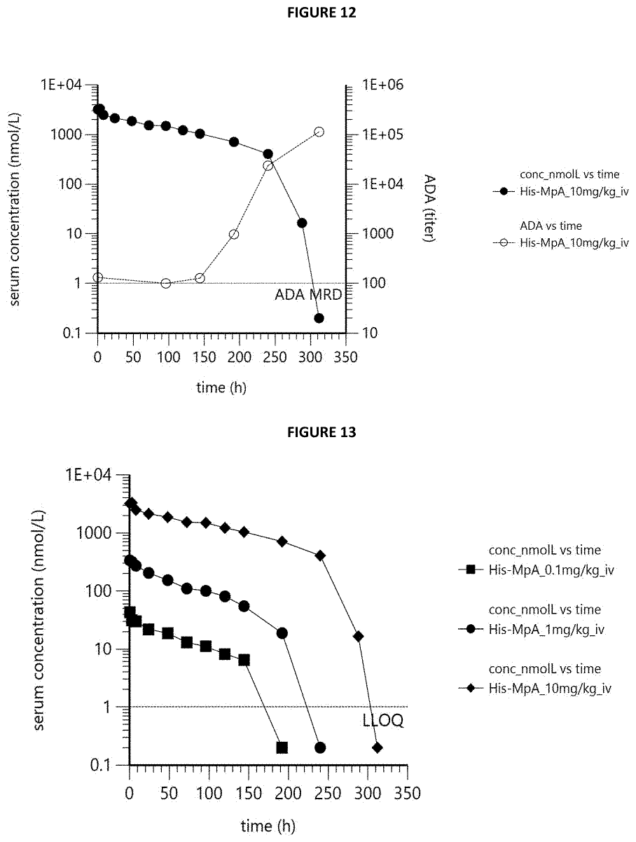

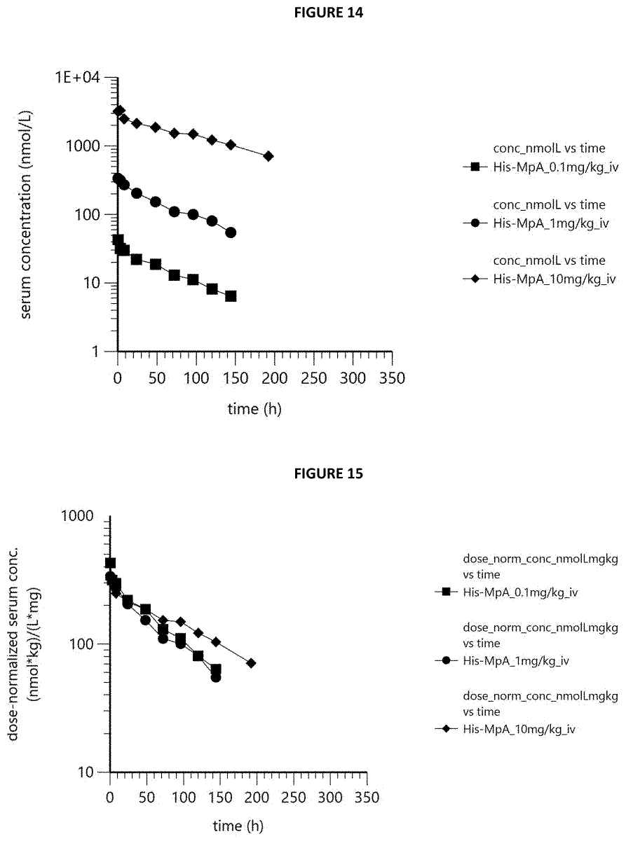

Serum concentration-time profiles of His-MpA in cynomolgus monkey (n=1) following a single intravenous dose of 0.1 mg/kg of the test compound given as 30 min infusions are shown in FIG. 10. Serum concentration-time profiles in combination with ADA titer-time traces in the same animals are also shown in FIG. 10. Comparable data sets for animals infused with 1 mg/kg or 10 mg/kg are shown in FIG. 11 and FIG. 12, respectively. Combined serum concentration-time profiles of animals receiving the different dose levels are shown in FIG. 13. Combined serum concentration-time profiles of animals receiving the different dose levels, for which data points assumed to be affected by ADAs were excluded, are shown in FIG. 14. Combined dose-normalized concentration-time profiles of animals receiving the different dose levels are shown in FIG. 15. No AMAs (or very low signal) were detected in pre-dose samples of the animals. Following intravenous infusion of His-MpAs at dose levels of 0.1, 1 and 10 mg/kg to animals, no increase of AMA titers was observed up to 144 h after administration. The onset of AMA generation in animals was observed between 144 h and 192 h in all dose groups. The rise in AMA titers was combined with a rapid loss of His-MpA exposure in the animals. Concentration-time data of animals which were assumed not to be affected by AMAs were used to calculate PK parameters by non-compartmental analyses. The parameters calculated for the animals following single intravenous infusion of 0.1, 1 and 10 mg/kg are given in Table 9.

TABLE 9
Pharmacokinetic characteristics of His-MpA in cynomolgus monkey
following single intravenous infusion of 0.1, 1 and 10 mg/kg

| | | Values | | |
|---|---|---|---|---|
| | | 0.1 mg/kg | 1 mg/kg | 10 mg/kg |
| Parameter | Unit | animal 1 | animal 2 | animal 3 |
| AUCinf | h*nmol/L | 2859 | 25825 | 405680 |
| AUCinf_D | (h*nmol*kg)/(L*mg) | 28590 | 25825 | 40568 |
| AUClast | h*nmol/L | 2250 | 20146 | 288444 |
| Cmax | nmol/L | 43 | 337 | 3300 |
| Cmax_D | (nmol*kg)/(L*mg) | 430 | 337 | 330 |
| Clast | nmol/L | 6.38 | 54.8 | 711 |
| Tmax | H | 0.167 | 0.167 | 3.0 |
| Cl_pred | L/h/kg | 0.00044 | 0.00049 | 0.00031 |
| Vss_pred | L/kg | 0.041 | 0.046 | 0.048 |
| t½ | H | 66 | 68 | 109 |
| AUC_% extrapol | % | 21 | 22 | 29 |

Concentration-time data points used to calculate half-life are highlighted in Table 10 with an asterisk for the dose levels 0.1, 1 and 10 mg/kg, respectively.

TABLE 10
Serum concentration-time data of His-MpA in cynomolgus monkey
following single intravenous infusion of 0.1, 1 and 10 mg/kg

| | Serum concentration (nmol/L) | | |
|---|---|---|---|
| Nominal | 0.1 mg/kg | 1 mg/kg | 10 mg/kg |
| time (hr) | animal 1 | animal 2 | animal 3 |
| 0.167 | 42.8 | 337 | 3200 |
| 3 | 31.5 | 318 | 3300 |
| 8 | 29.8 | 273 | 2480 |
| 24 | 22.0* | 205* | 2130* |
| 48 | 18.7* | 154* | 1870* |
| 72 | 13.0* | 110* | 1530* |
| 96 | 11.1* | 101* | 1490* |
| 120 | 8.11* | 80.9* | 1220* |
| 144 | 6.38* | 54.8* | 1040* |
| 192 | BLQ | 18.8** | 711* |
| 240 | BLQ | BLQ | 407** |
| 288 | BLQ | BLQ | 16.6** |
| 312 | BLQ | BLQ | BLQ |

*values used for calculation of half-life (t½);
**values assumed to be affected by ADA, excluded for analyzes
BLQ: Below Limit of Quantitation

Single intravenous administration of His-MpA to the animals at dose levels of 0.1, 1 and 10 mg/kg led to Cmax values observed 10 min to 3 h after the end of the 30 min infusion which increased proportionally to an increase in dose (43, 337 and 3300 nmol/L, respectively). Subsequently, the courses of the concentration-time traces were similar for the different dose levels and were characterized by a rapid initial decline of the serum concentrations which lasted up to approximately 24 h post compound administration followed by a roughly mono-exponential decline up to 144 h (0.1 and 1 mg/kg) or 192 h (10 mg/kg). Half-life values in the mono-exponential phase were calculated to be 66 h (2.8 days), 68 h (2.8 days) and 109 h (4.5 days) for the animals dosed with 0.1, 1 and 10 mg/kg, respectively. Whereas Cmax values increased with dose in an approximately dose-proportional manner, exposure (AUCinf) increased dose-proportionally between 0.1 and 1 mg/kg (2859 and 25825 h*nmol/L, respectively) but slightly more than dose proportionally between 1 and 10 mg/kg (25825 and 405680 h*nmol/L, respectively). This results in a lower clearance value for the 10 mg/kg animal (0.00031 L/h/kg) compared to the animals receiving the lower dose levels (0.00044 and 0.00049 L/h/kg). The non-dose-linear pharmacokinetic behavior between 1 and 10 mg/kg is apparent in the concentration-time profiles of His-MpA with shorter half-lives observed for the 0.1 and 1 mg/kg dose but longer half-life for the higher dose. Values determined for volume of distribution (0.041, 0.046 and 0.048 L/kg) indicate that His-MpA is largely confined to the systemic circulation of the animals.

### Conclusion:

The pharmacokinetic analysis of His-MpA in cynomolgus monkeys reveals that Cmax values increased with dose in an approximately dose-proportional manner following single 30 min infusions of His-MpA at dose levels of 0.1, 1 and 10 mg/kg. Exposure (AUCinf) increased dose-proportionally between 0.1 and 1 mg/kg but slightly more than dose-proportionally between 1 and 10 mg/kg. The non-dose-linear pharmacokinetic behavior between 1 and 10 mg/kg is apparent in the concentration-time profiles of His-MpA with shorter half-life observed for the 0.1 and 1 mg/kg dose (2.8 days) but longer half-life for the higher dose (4.5 days). Values determined for volume of distribution indicate that His-MpA is largely confined to the systemic circulation of the animals.

Pharmacokinetic parameters of His-MpA derived from the described study are based on data from single monkeys (n=1) in each of the dose groups. Animals generated AMAs with an onset between 144 h and 192 h following compound infusion. The rise of AMA titers was associated with a loss of His-MpA exposure in the animals.

### Example 7—Monitoring T Cell Activation by Multispecific Protein a (MpA) In Vivo

4-1BB (CD137) is a co-stimulatory receptor, which belongs to the TNF receptor superfamily, and is expressed on a multitude of cells of the hematopoietic lineage. Most relevantly, 4-1BB is transiently upregulated on CD8+ T cells following activation, but can also be expressed on NK cells and activated CD4+ helper T cells as well as many other types of lymphocytes and activated endothelium.

The effects of treatment with MpA on memory T cell activation and proliferation were assessed and compared to anti-4-1BB mAb MOR-7480 upon a single intravenous administration to cynomolgus monkeys. Whole blood samples were collected throughout the in-life part of this study and changes in the T cell compartment were monitored by flow cytometric analysis.

### Methods:

Sample Processing and Staining:

2×100 μl of whole blood sample from cynomolgus monkeys was aliquoted and stained with panel 1 or panel 2, respectively. Panel 1 and panel 2 were composed of staining antibody mix. Firstly, all samples were incubated with Purified NA/LE Human BD Fc Block (1:200) for 10 minutes at RT, followed by addition of 50 μl of BD Horizon Brilliant Stain Buffer per sample. Next, samples were incubated with antibody mix from panel 1 or panel 2, respectively, for 30 minutes in the dark. Samples were then lysed by adding 2 mL of 1×FACS lysing solution and incubated for 15 minutes at room temperature in the dark. After 2 washes in staining buffer, 250 μl of Cytofix/Cytoperm solution was added for fixation and permeabilization of the cell membrane, and incubated for 20 minutes at 4° C. in the dark. After incubation, 3 mL of 1× Perm/wash buffer was added and centrifuged for 5 minutes at 500×g at 4° C. An additional wash with 1 mL of 1× Perm/wash buffer was performed. Finally, samples were resuspended in 300 μl Stain buffer.

Sample Acquisition:

Samples were measured at the FACSLyric instrument (Serial No. R659180000061), BD Biosciences.

Data Analysis:

Raw .fcs with compensation matrix were evaluated using FlowJo software version 10.0.2. Summary plots are generated using Graph Pad Prism software, version 7.

### Results and Discussion

Study outline: One female cynomolgus monkey per group was infused with His-MpA or anti-4-1BB mAb MOR-7480 as depicted in Table 11.

TABLE 11
Study Design of in-life phase of the animal study

| | Test/Reference | Test/Reference | Animal reference |
|---|---|---|---|
| Group | Item | item dose [mg/kg] | number |
| 1 | His-MpA | 0.1 | 1 |
| 2 | | 1 | 2 |
| 3 | | 10 | 3 |
| 4 | anti-4-1BB mAb | 10 | 4 |
| | MOR-7480 | | |

At three defined time points (1. Pre-dose, 2. Day 6, and 3. Day 13) 1 ml of whole blood was collected from each of the monkeys in K₃EDTA coated tubes to prevent coagulation. The whole blood samples were transferred to BioAgilytix/IPM at room temperature within 3 hours of sampling, for further analysis.

Results of the Assessment of Collected Whole Blood Samples:

To assess proliferation among memory T cells, blood samples were isolated from cynomolgus monkeys at several time points following single intravenous doses of His-MpA or anti-4-1BB mAb MOR-7480. Using flow cytometry, memory subsets of CD4 T cells and CD8 T cells were identified using CD95 and CD28 as markers of central memory (CD95+ CD28+) and effector memory (CD95+ CD28−) subsets as well as naïve T cells (CD95− CD28−) in macaques. Ki-67 was analyzed as a proliferation marker; CD25, CD69 and 4-1BB were analyzed as T cell activation markers as described previously (Fisher et al., Cancer Immunology and Immunotherapy, 61:1721-1733, 2012).

Gating strategy: Lineage markers CD4, CD8, CD16, CD28 and CD95 were used to define target cell populations, as follows:
1. Central memory T cell (Tcm): CD8(or CD4)+CD28+CD95+
2. Effector memory T cell (Tem): CD8(or CD4)+CD28−CD95+
3. Naïve T cell (Tn): CD8(or CD4)+CD28−CD95−

On each of the target cell populations, expression of the following specific markers was monitored in order to assess cell proliferation (Ki-67 antigen) or activation (4-1BB, CD25, CD69).

Anti-4-1BB mAb MOR-7480, but not his-MpA, Increased Proliferation of the T Cell Memory Compartment in the Blood:

Analysis of the fresh blood samples at three defined time points showed significant expansion of CD8+ Tcm and Tem at day 13 (data not shown), in the animals from the anti-4-1BB mAb MOR-7480 treated group. CD4+ Tcm and Tem cells in that group also showed some expansion, but to a lesser extent than the CD8+ cells. This expansion was not observed in any of the His-MpA-treated groups.

No Increase in the Expression of CD25 and CD69 Activation Markers, was Detected Following Treatment with Either of the Compounds:

Analysis of CD25 expression on CD8+ and CD4+ memory T cell compartments did not show any significant change compared to the baseline (pre-dose time point) in any of the treatment groups. As expected, CD25 expression on CD8+ cells was negative or low, while CD4+ cells showed moderate but constant CD25 expression in all groups. CD69 was not expressed on any of the CD4 or CD8 subsets throughout the study (not shown).

### Conclusion

Healthy cynomolgus monkeys were dosed with His-MpA or the 4-1BB agonistic mAb MOR-7480 and the expression of T cell proliferation and activation markers was monitored by flow cytometry. His-MpA, in contrast to anti-4-1BB mAb MOR-7480, did not induce proliferation of CD8+ or CD4+ Tcm and Tem cells.

### Example 8—MpB Demonstrated Anti-Tumor Activity In Vivo

The following Example evaluated the dose-dependent efficacy of repeated doses of the Multispecific binding protein B (MpB), a mouse surrogate for MpA comprising a FAP binding domain that binds mouse FAP and 4-1BB binding domains that bind human 4-1BB, in comparison to the anti-4-1BB agonistic antibody 20H4.9 and an anti-FAP-4-1BBL fusion protein in the HT-29 colon carcinoma xenograft model reconstituted with human PBMCs (MiXeno). This humanized mouse model has been described to offer a suitable proof of concept for testing immune stimulatory efficacy of immune checkpoint and co-stimulatory drugs such as agonistic anti-4-1BB antibodies. Treatment with anti-4-1BB mAb 20H4.9 in this model is sufficient to significantly slow tumor growth. However, it also induces strong systemic effects such as accelerated graft versus host disease (GVHD) and liver T cell infiltration resulting in premature death compared to untreated mice. The model was used to assess whether MpB was able to increase intra-tumoral T cell infiltration and slow tumor growth while avoiding some of the non-tumor effects produced by anti-4-1BB mAb 20H4.9, a non-targeted 4-1BB agonistic monoclonal antibody. A FAP-targeted natural 4-1BB ligand was included in the study for comparison.

### Materials and Methods:

Tumor Experiment:

Immunodeficient NOG mice were inoculated subcutaneously in the right flank region with HT-29 tumor cells (3.5×10⁶). The mice were then humanized by injecting peripheral blood mononuclear cells (PBMCs) from two healthy human donors (3.5×10⁶ cells/mouse). The test articles were administered to the tumor-bearing mice according to the predetermined regimen as shown in Table 12.

TABLE 12
Study design - experimental groups.

| | | Inoculum | | | | | |
|---|---|---|---|---|---|---|---|
| | | | PBMC | | | | |
| | | HT-29 | 7 × 10⁶/ | | Dose | Dosing | Actual |
| Group | N | (s.c.) | mouse (i.p.) | Treatment | (mg/kg) | Route | Schedule |
| 1 | 5 | 3.5 × 10⁶ | Donor 1 | Vehicle | 0 | i.v. | Q2D×10 |
| | 5 | cells | Donor 2 | | | | |
| 2 | 5 | | Donor 1 | Anti-4-1 BB mAb | 8 | i.v. | Q6D×3 |
| | 5 | | Donor 2 | 20H4.9 | | | |
| 3 | 5 | | Donor 1 | Anti-FAP-4-1BBL | 4 | i. v. | Q6D×3 |
| | 5 | | Donor 2 | | | | |
| 4 | 5 | | Donor 1 | MpB | 8 | i.v. | Q2D×10 |
| | 5 | | Donor 2 | | | | |
| 5 | 5 | | Donor 1 | MpB | 1.6 | i.v. | Q2D×10 |
| | 5 | | Donor 2 | | | | |
| 6 | 5 | | Donor 1 | MpB | 0.32 | i.v. | Q2D×10 |
| | 5 | | Donor 2 | | | | |

Date of tumor cell and PBMC inoculation was denoted as day 0. Tumor growth was monitored every 3 to 4 days. On day 18 of the experiment, mice were sacrificed, tumors removed, and studied by flow cytometry and quantitative immunofluorescence (QIF). Tumor growth analyses were limited to 18 days because mice started to show signs of GVHD after this time.

Flow Cytometry:

Data from raw FCS files were analyzed with FlowJo software (TreeStar). Cells were gated on live lymphocytes expressing the human surface markers CD45, CD4 and CD8. Dead cells were excluded from the analysis via incorporation of the live-dead labelling dye 7-AAD. The percentages of human CD8 T cells as percentage of total human CD45 positive cells detected in blood were determined.

Immunohistochemistry:

Tissues were recovered from mice at necropsy, and embedded in optimum cutting temperature compound (Sakura) and frozen without prior fixation. OCT embedded cryo-preserved specimens were cut into 7 μm sections and mounted on glass slides. The slides were fixed with cold acetone. The multiple immunofluorescence staining was performed with the following antibodies: anti-CD4 (Goat Pab, R&D System #AF-379-NA), anti-CD8 (Rabbit PAb, Abcam #ab40555) and anti-CD45 (clone H130, Biolegend #304002). These antibodies were respectively detected by anti-Sheep-Alexa Fluor® 647 (Thermofisher #A21448), anti-rabbit-Rhodamine Red™-X (Jackson ImmunoResearch #711-296-152) and anti-mouseIgG1-Alexa Fluor® 488 (Jackson ImmunoResearch #115-545-205). The images were acquired on a Zeiss Axio Scan.Z1 slidescanner. The images were transferred with Zen blue software and analyzed using ImageJ 1.51n software, with FIJI package to quantify numbers of human CD45, CD8 and CD4 T cells.

Statistical Analysis:

Statistical analyses were performed with the Prism 7.0.2 software (GraphPad Software). Tumor growth and body weight data were analyzed for statistically significant differences by using repeat measurement two-way ANOVA and Tukey's multiple comparison test (GraphPad Prism, Vers. 7.02). Survival curves were analyzed by the Kaplan-Meier method and compared by log-rank tests. Flow cytometry data at study end were analyzed using 1-way ANOVA (GraphPad Prism, Vers. 7.02). A two-tailed P<0.05 was considered as statistically significant.

### Results

Tumor Growth:

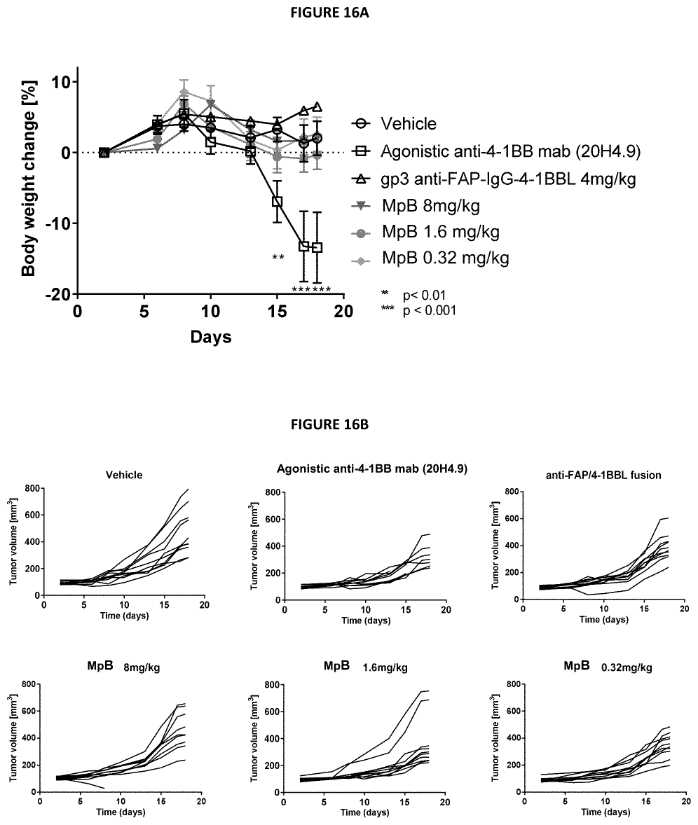

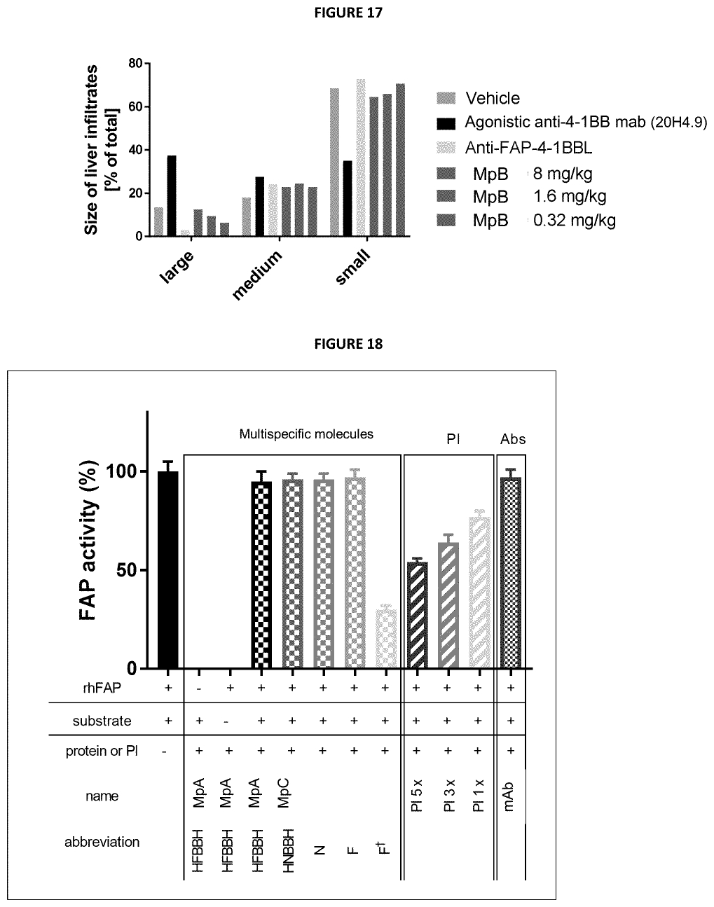

Tumor growth was followed individually over time (FIGS. 16 and 17). The mean tumor growth curves of different treatment groups are shown in FIG. 16. Tumor growth curves of individual mice are shown in FIG. 17.

In addition to the statistical analysis conducted on the data obtained at day 18 after tumor inoculation using Independent-Samples T test, tumor growth data were analyzed for statistically significant differences by using repeat measurement two-way ANOVA followed by Tukey's multiple comparison test. The tumor growth inhibition is summarized in Table 13.

TABLE 13
Summary of Anti-tumor Activity of Treatment

| | | Tumor Size | | | |
|---|---|---|---|---|---|
| | | at day 18 | TGI | Signif- | P |
| Group | Treatment | (mm³)^{a} | (%) | icance^{b} | value^{b} |
| 1 | Vehicle | 477 ± 56 | — | | — |
| 2 | Anti-4-1BB mAb | 326 ± 33 | 32 | ** | 0.0032 |
| | 20H4.9 (8 mg/kg) | | | | |
| 3 | Anti-FAP/4-1BBL | 392 ± 32 | 18 | ns | 0.1902 |
| | (4 mg/kg) | | | | |
| 4 | MpB (8 mg/kg) | 462 ± 47 | 3 | ns | 0.9986 |
| 5 | MpB (1.6 mg/kg) | 366 ± 61 | 23 | * | 0.0338 |
| 6 | MpB (0.32 mg/kg) | 349 ± 28 | 27 | ** | 0.0075 |

^{a}Mean ± SEM;
^{b}RM two-way ANOVA over all time points of tumor growth curves followed by Tukey's multiple comparison test vs. vehicle control (* p < 0.05, ** p < 0.001).

Analysis of the entire tumor growth curves gives higher power to the analysis compared to analysis of only the final tumor volume at the end of the study. The two analyses correlate well, except that the two-way ANOVA analysis revealed statistical significance for the MPB 1.6 mg/kg group in addition to anti-4-1BB mAb 20H4.9 and MpB 0.32 mg/kg groups. Tumor growth was delayed in the anti-4-1BB mAb 20H4.9 (p<0.001) and the MpB 1.6 mg/kg (p<0.05) and 0.32 mg/kg treatment groups (p<0.001), but not in the 8 mg/kg MPB or the anti-FAP-4-1BBL fusion groups. Anti-FAP-4-1BBL fusion showed a trend to inhibit tumor growth as well but the effect was not statistically significant. The vehicle administered had no significant impact on tumor growth. In summary, the test substances, anti-4-1BB mAb 20H4.9 and MpB, demonstrated significant anti-tumor activities in the subcutaneous HT-29 human colon cancer MiXeno model.

Immunophenotyping of Blood and Tumors:

To confirm the results obtained by flow cytometry, the human CD4 and CD8 T lymphocyte density was analyzed by histology in tumors excised on day 18. Histological examination was performed using tissues from 5 mice per group (data not shown). Treatment with MpB led to denser infiltrates of human CD8 T lymphocytes in comparison with the vehicle group. While all doses of MpB tested as well as anti-FAP-4-1BBL showed this trend, the difference reached significance only for the MpB 0.32 mg/kg group (P<0.01; FIG. 18A). On the other hand, numbers of CD4 tumor infiltrating lymphocytes were not significantly different across all groups.

Graft Versus Host Disease:

GVHD is considered a valuable model to test immunomodulatory strategies, where the engrafted human T lymphocytes are amenable for regulation by therapeutic agents. After 3 to 4 weeks, engrafted T lymphocytes start to infiltrate the spleen, liver, and lung with signs of serious tissue damage after 4 to 5 weeks. Treatment with anti-4-1BB agonistic antibodies accelerates and exacerbates GVHD caused by the adoptive transfer of human T lymphocytes.

Accordingly, mice injected with 3.5×10⁷ human PBMCs started to lose weight in the anti-4-1BB mAb 20H4.9 treated group at 2 weeks after adoptive transfer (data not shown) and by day 18 in most instances needed to be sacrificed according to predefined animal health termination criteria due to body weight loss and signs of respiratory distress, hunched posture, and/or fur loss. Mice in the vehicle group maintained their body weight over the 18 day study duration and all the other treatment groups did not show a significant reduction in body weight compared to the vehicle group. The anti-4-1BB mAb 20H4.9 treated group showed a significant decrease in body weight from day 15 onwards (P<0.01 on day 15 and p<0.001 on days 17 and 18) compared to the vehicle control (data not shown). Mice were followed for lethal GVHD and a significant decrease in overall survival was observed in the anti-4-1BB mAb 20H4.9 group as compared with the control group. None of the mice in the control group died and no significant body weight loss was observed. Six of ten (60%) mice in the anti-4-1BB mAb 20H4.9-treated group showed strong signs of GVHD and either died or reached the termination criterion of ≥20% body weight loss and were sacrificed. One of 30 (3%) mice died in the MpB groups but none of the animals showed body weight loss of greater than 20% (p<0.001, Log-rank test). Except for one mouse in group 4 (MpB, 8 mg/kg) which was found dead early in the study (after 1 week of treatment), all the mice in the other groups survived until the study end on day 18 (data not shown).

Treatment with MpB did not lead to aggravation of GVHD despite the fact that PBMC phenotyping results indicated a certain degree of CD8 T cell expansion in peripheral blood. On the other hand, treatment with anti-4-1BB mAb 20H4.9 led to accelerated GVHD with significantly increased body weight loss and mortality compared to MpB and vehicle control.

In summary, treatment with anti-4-1BB mAb 20H4.9 adversely affected body weight and overall survival due to accelerated onset of GVHD towards the end of the study (15 days post tumor cell/PBMC inoculation). Treatment with anti-FAP-4-1BBL fusion or MpB did not lead to body weight loss and reduced survival compared to the vehicle group despite showing similar anti-tumor activities compared to treatment with anti-4-1BB mAb 20H4.9.

Histological Analysis of Liver T Cell Infiltration:

Histologic examination of livers excised on day 18 was performed using tissues from 5 mice per group. Administration of anti-4-1BB mAb 20H4.9 induced increased liver T cell infiltration by human PBMCs in NOG mice, similar to published results. Quantification of infiltrates categorized as small, medium and large by surface area confirmed that anti-4-1BB mAb 20H4.9 induced significantly larger area infiltrates compared to the vehicle group and that treatment with the FAP-targeted 4-1BB agonists MpB and anti-FAP-4-1BBL fusion protein did not induce an increase in liver T cell infiltration (FIG. 18).

### Conclusion

The test substances, anti-4-1BB mAb 20H4.9 and MpB, demonstrated anti-tumor activity in the subcutaneous HT-29 human colon cancer MiXeno model. Treatment with MpB also led to an increased density of human CD8 T cells in the tumor compared to vehicle-treated mice. There was no clear dose response of MpB over the tested range from 0.32 to 8 mg/kg since all doses produced similar increases in human CD8 T cell percentages whereas the anti-tumor effect was significant for the lower two doses but not the 8 mg/kg dose. Treatment with anti-4-1BB mAb 20H4.9 adversely affected body weight and overall survival due to accelerated onset of graft versus host disease (GVHD) towards the end of the study. Treatment with anti-4-1BB mAb 20H4.9 also led to increased human T cell infiltration into the liver. In contrast, treatment with MpB and anti-FAP-4-1BBL fusion protein was well tolerated and did not lead to body weight loss or reduced survival and did not produce increased liver T cell infiltration compared to the vehicle group.

In summary, MpB treatment produced similar antitumor activity to treatment with anti-4-1BB mAb 20H4.9 in the HT-29 humanized xenograft model. In contrast to treatment with anti-4-1BB mAb 20H4.9, MpB increased the percentage of human CD8+ intra-tumoral lymphocytes, did not aggravate GVHD, did not induce significant body weight loss and did not increase infiltration of T cells into the liver.

### Example 9: FAP/4-1BB Bi-Specific Ankyrin Repeat Proteins Bind to Cells and Activate 4-1BB Signaling Via FAP-Mediated Clustering

Agonist-mediated clustering of 4-1BB on the cell surface is thought to be required or at least highly beneficial for effective activation of 4-1BB signaling. To test whether such clustering and activation of 4-1BB can also be mediated by FAP/4-1BB bispecific molecules, various FAP/4-1BB-specific constructs were tested.

The FAP/4-1BB bispecific constructs were cloned by digesting the sequences encoding monovalent 4-1BB binding domains with BamHI and HindIII and a vector (pMPCME298) providing sequences encoding the FAP binding domain and a peptide linker (SEQ ID NO:4), as well as an N-terminal His-tag (SEQ ID NO: 56) to facilitate simple protein purification, with BsaI and HindIII. The vector and an insert encoding a monovalent 4-1BB binding domain were then ligated and transformed into inducible E. coli bacteria. Three clones per construct were sequenced by Microsynth. Correct clones where then expressed and purified using benchtop purification with two triton wash steps.

The following FAP-4-1BB bispecific constructs were generated for further functional testing:
bispecific construct #44 (SEQ ID NO:44 with a His-tag (SEQ ID NO:43) fused to its N terminus; comprising SEQ ID NO:3 as 4-1BB binding domain);
bispecific construct #45 (SEQ ID NO:45 with a His-tag (SEQ ID NO:43) fused to its N terminus; comprising SEQ ID NO:51 as 4-1BB binding domain);
bispecific construct #46 (SEQ ID NO:46 with a His-tag (SEQ ID NO:43) fused to its N terminus; comprising SEQ ID NO:52 as 4-1BB binding domain);
bispecific construct #47 (SEQ ID NO:47 with a His-tag (SEQ ID NO:43) fused to its N terminus; comprising SEQ ID NO:53 as 4-1BB binding domain);
bispecific construct #48 (SEQ ID NO:48 with a His-tag (SEQ ID NO:43) fused to its N terminus; comprising SEQ ID NO:54 as 4-1BB binding domain); and
bispecific construct #49 (SEQ ID NO:49 with a His-tag (SEQ ID NO:43) fused to its N terminus; comprising SEQ ID NO:55 as 4-1BB binding domain).

As a negative control, an ankyrin repeat domain (SEQ ID NO: 57) with no binding specificity for human 4-1BB was cloned into the vector, resulting in bispecific construct #50: bispecific construct #50 (SEQ ID NO:50 with a His-tag (SEQ ID NO:56) fused to its N terminus; comprising SEQ ID NO:57 instead of a 4-1BB binding domain).

FAP-41BB Bispecific Constructs Bind to 4-1BB and FAP with High Affinity

The FAP-41 BB bispecific constructs were investigated with SPR to obtain accurate affinity data for human 4-1BB, cyno 4-1BB and human FAP targets.

Assay Set-Up:

Briefly, SPR measurements were performed using a ProteOn XPR36 instrument (BioRad) as described above. Biotinylated human and cynomolgus 4-1BB and human FAP were immobilized either directly or indirectly via NeutrAvidin (˜6000 RU pre-coated) on a GLC chip to reach 600 RU, 700 RU and 2000 RU, respectively. The interaction of FAP-4-1BB bispecific constructs to the coated targets was measured by injecting the bispecific constructs in a serial dilution of 50, 16.7, 5.6, 1.9 and 0.6 nM with an association of 120 s and dissociation of 1800 s using a constant flow rate of 30 μl/min. Targets were regenerated between the individual measurements using 10 mM glycine pH 2 and 124 mM H₃PO₄. The signals were double referenced against the running buffer (PBST) treated control lane.

Screen:

The results of the SPR measurements of FAP-4-1BB bispecific constructs are summarized in Table 15. The FAP-4-1BB bispecific constructs showed binding affinities of 0.4 to 1.5 nM to human 4-1BB, 1.1 to 2.9 nM to cynomolgus 4-1BB and 0.1 to 0.4 nM to human FAP. The FAP-4-1BB bispecific constructs bound with higher affinity to hFAP than to h4-1BB. Cross-reactive binding to cynomolgus 4-1BB was confirmed for all tested FAP-4-1BB bispecific constructs, with at most about a 4-fold difference in binding affinity as compared to human 4-1BB.

TABLE 15
K_{D} values of FAP-41BB bispecific molecules interactions
with h4-1BB, c4-1BB and hFAP

| | K_{D} [nM]- | K_{D} [nM]- | K_{D} [nM]- |
|---|---|---|---|
| bispecific construct # | h4-1BB | c4-1BB | hFAP |
| bispecific construct #44 | 1.45 | 1.12 | 0.36 |
| bispecific construct #45 | 1.47 | n.d. | 0.33 |
| bispecific construct #46 | 1.15 | n.d. | 0.27 |
| bispecific construct #47 | 0.77 | 2.90 | 0.07 |
| bispecific construct #48 | 0.44 | n.d. | 0.17 |
| bispecific construct #49 | 0.86 | 1.48 | 0.17 |
| bispecific construct #50 | No binding | No binding | 0.22 |

### FAP-4188 Bispecific Constructs Activate 4-1BB Signaling in 4-18B-Expressing Cells Mediated by FAP-Induced Clustering

The FAP-41 BB bispecific constructs were then further tested for their ability to activate 4-1BB signaling in 4-1BB-expressing cells mediated by clustering via FAP-binding.

Assay Setup:

HT1080 cells expressing human-4-1BB and an NF-κB-luciferase reporter gene (see Example 1) were harvested and resuspended in MEMα medium+Glutamax supplied with 10% (v/v) FBS, 1% PenStrep, 1 mg/mL G418, 100 μg/mL Normocin and 100 μg/mL Zeocin. Using 96-well plates, 10,000 h4-1BB-HT1080-luciferase cells were plated together with human FAP-coated beads and increasing concentrations of bispecific constructs in the presence of FAP-biotin-coated streptavidin beads. Plates were incubated at 37° C., 5% CO₂ for 20 hours. Then supernatants were collected and centrifuged in a fresh 96-well plate. QUANTI-Luc reagent (Invivogen, Cat. No. rep-qlc1) was mixed with the supernatant and luminescence read on a Tecan M1000 luminescence plate reader. EC₅₀ values were determined by fitting the data with the four-parameter logistical fit model using Graphpad Prism software (version 7.02).

Results:

This h4-1BB-HT1080 luciferase reporter assay demonstrated that the disclosed bispecific constructs show 4-1BB agonism in the presence of FAP-coated beads. The 4-1BB agonism was dependent on FAP-mediated clustering, since in the absence of FAP-coated beads or in the absence of a FAP binding domain in the bispecific construct, no agonism was observed. Table 16 provides the EC₅₀ values of the bispecific constructs:

TABLE 16
4-1BB activation in HT1080 cells in presence of FAP-beads:
EC₅₀ values of FAP-4-1BB bispecific constructs

| bispecific construct # | EC50 [nM] |
|---|---|
| bispecific construct #44 | 4.59 |
| bispecific construct #45 | 5.15 |
| bispecific construct #46 | 5.85 |
| bispecific construct #47 | 6.19 |
| bispecific construct #48 | 3.71 |
| bispecific construct #49 | 6.52 |

The FAP-4-1BB bispecific constructs were further tested for their ability to activate 4-1BB signaling in 4-1BB-expressing cells mediated by clustering via FAP-binding, using an assay that measures NF-κB reporter gene activation in 4-1BB-expressing HT1080 cells co-cultured in the presence of FAP-expressing cells.

### Generation of CHO Cells Expressing Human FAP

In brief, an expression vector (pMPMPA13) was generated by standard molecular biology techniques using a cDNA encoding human FAP. Chinese hamster ovary (CHO) cells (ATCC® CCL-121™) were transfected with the expression vector using Lipofectamine. Selection pressure was applied using different concentrations of Geneticin G-418 (Promega, V8091). Expression of hFAP was analysed by flow cytometry using an anti-FAP antibody. Two different populations (Population 1 and Population 2) of CHO-FAP transfectants were chosen for further experiments. FACS analysis demonstrated that CHO-FAP cells but not wildtype CHO cells (CHO-wt) express hFAP on the cell surface (data not shown).

Assay Setup:

h4-1BB-HT1080-luciferase cells as well as CHO-FAP cells were harvested and resuspended in MEMα medium+Glutamax supplied with 10% (v/v) FBS, 1% PenStrep, 1 mg/mL G418, 100 μg/mL Normocin™ and 100 μg/mL Zeocin™. Using 96-well plates, 40,000 h4-1BB-HT1080-luciferase cells and 40,000 CHO-FAP cells were plated and increasing concentrations of FAP-4-1BB bispecific constructs were added to the cells and incubated at 37° C., 5% CO₂. After 20 hours, supernatants were collected and centrifuged in a fresh 96-well plate. QUANTI-Luc reagent (Invivogen, Cat. No. rep-qlc1) was mixed with the supernatant and luminescence read on a Tecan M1000 luminescence plate reader. EC₅₀ values were determined by fitting the data with the four-parameter logistical fit model using Graphpad Prism software (version 7.02).

Results:

The results showed that in the presence of FAP-expressing cells (Population 1), all FAP-4-1BB bispecific constructs induced 4-1BB signaling in the 4-1BB-expressing cells to a comparable extent, mediated by clustering via FAP-binding. Bispecific construct #50, which does not bind to 4-1BB, had no effect on 4-1BB signaling. Table 17 provides the EC₅₀ values of the FAP-4-1BB bispecific constructs:

TABLE 17
4-1BB activation in HT1080 cells in presence of
FAP-expressing CHO cells (Population 1):
EC₅₀ values of FAP/4-1BB bispecific constructs

| bispecific construct # | EC50 [nM] |
|---|---|
| bispecific construct #44 | 0.47 |
| bispecific construct #45 | 0.67 |
| bispecific construct #46 | 0.73 |
| bispecific construct #47 | 0.36 |
| bispecific construct #48 | 0.28 |
| bispecific construct #49 | 0.91 |

Similar results were obtained with the second population of FAP-expressing cells (Population 2). Table 18 provides the E050 values of the bispecific constructs:

TABLE 18
4-1BB activation in HT1080 cells in presence of
FAP-expressing CHO cells (Population 2):
EC₅₀ values of FAP/4-1BB bispecific constructs

| bispecific construct # | EC50 [nM] |
|---|---|
| bispecific construct #44 | 0.36 |
| bispecific construct #45 | 0.39 |
| bispecific construct #46 | 0.37 |
| bispecific construct #47 | 0.18 |
| bispecific construct #48 | 0.16 |
| bispecific construct #49 | 1.04 |

In conclusion, all tested FAP/4-1BB bispecific constructs were able to activate 4-1BB signaling in 4-1BB-expressing cells mediated by clustering via FAP-binding.

### Example 10: Protease Activity of FAP in Presence or Absence of Multispecific Proteins

This example describes a FAP activity assay that was performed in the presence of various FAP/4-1BB binding molecules to determine whether the intrinsic FAP enzymatic activity is inhibited upon binding of the multispecific recombinant proteins.

FAP is a type II single transmembrane serine protease whose expression is highly upregulated on sites of tissue remodeling like tumors (e.g. expressed at the surface of stromal fibroblasts in >90% of epithelial cancers), wound healing, embryonic tissue and sites of inflammations (e.g. atherosclerosis/arthritis), while FAP expression is difficult to detect in non-diseased adult organs. This atypical serine protease has both dipeptidyl peptidase (exopeptidase) and endopeptidase activities, cleaving substrates at a post-proline bond. Structurally, FAP consists of a short cytoplasmic N-terminal sequence (4 aa), a single transmembrane helix (21 aa) and an extracellular domain (735 aa) which forms an eight bladed β-propeller and a α/β-hydrolase domain. FAP is active as a homodimer. The catalytic triad, essential for FAP protease activity, is composed of residues Ser624, Asp702 and His734. The active site is accessible either through the central hole of the beta-propeller or through a cavity at the interface of the beta-propeller and the hydrolase domain.

The protease activity of FAP produces cleavage of a variety of substrates, including neuropeptide Y, type I collagen and α2-antiplasmin but also the substrate Z-GLY-PRO-AMC, which can be cleaved by both the exopeptidase or endopeptidase activity into a product that can be measured with a fluorescence reader.

Molecules tested in the FAP activity assay are summarized in Table 19.

TABLE 19
Recombinant proteins used in the assay

| Molecule | | Format and |
|---|---|---|
| No | Name | Description |
| 1 | MpA | HFBBH |
| 2 | MpC | HNBBH, multispecific |
| | | negative control |
| 3 | FAP-binding domain | F |
| | only (F) | |
| 4 | Alternative FAP-binding | F† |
| | domain (F†) | |
| 5 | Ankyrin repeat domain, | N |
| | negative control (N) | |
| 6 | Benchmark (mAb) | FAP-binding antibody |

H Albumin binding domain
F Human FAP-binding domain
F† Alternative hFAP-binding domain
B Human 4-1BB binding
N Negative control (ankyrin repeat domain that does not bind to a target)

FAP Activity Assay.

The rhFAP target was diluted to 0.22 μg/ml in the assay buffer (50 mM Tris, 1 M NaCl, 1 mg/ml BSA, pH 7.5) and 45 μl per well was added to a 96 well plate (final concentration. 0.03 μg/ml (0.3 nM)). Molecules 1-5, as shown in Table 19, were applied at a 450-fold molar excess by adding 5 μl 2.7 μM molecules to the target sample (final concentration 135 nM). The benchmark anti-FAP antibody (molecule No. 6) was applied at the same concentration as molecules Nos. 1-5 (final concentration 135 nM). A protease inhibitor (PI) mixture (eComplete, EDTA-free from Merck) was used at different dilutions to show inhibition of the FAP activity. The rhFAP/protein or rhFAP/PI mixture was incubated for 90 minutes at 300 rpm before adding 50 μl 100 μM Z-GLY-PRO-AMC substrate (final concentration 50 μM). Prior to measurement, the plate was centrifuged for 2 min at 4000 rpm to remove any assay interfering bubbles. The fluorescence was measured every 5 min over a period of 95 min at 380 nm excitation and 460 nm emission using a fluorescence reader with a manual gain set at 105%. The time point after 45 minutes was used for the analysis with a signal to noise ratio larger than 70. FAP activity (given in %) was normalized to the assay controls of 100% activity (FAP and substrate, without molecules Nos. 1-6) and 0% activity (FAP, no substrate in presence of MpA). Quadruplet measurements were performed and illustrated as mean and standard deviation.

Results.

In a first step, dose response curves were measured using FAP concentrations from 0.01 nM up to 1.2 nM at fixed substrate concentration of 50 μM. A linear time-dependent signal increase was observed at an rhFAP target concentration of 0.3 nM over a time period of 95 min (measured every 5 minutes with an R² of 0.999). To determine the effect of protein binding on the enzymatic activity of FAP, the assay was conducted under FAP saturating conditions by adding the FAP-binding-molecule (e.g. MpA at 135 nM) in a 450-fold molar excess over FAP (0.3 nM) and more than 300-fold above the binding affinity of MpA against human FAP (K_{D}=0.4 nM).

As summarized in FIG. 18, MpA, MpC, and “F” (molecules Nos. 1-3) did not inhibit the intrinsic dipeptidyl FAP activity. The results are comparable to the benchmark mAb (molecule No. 6), an anti-FAP antibody which also showed no interference with the protease activity of FAP upon binding. Partial FAP activity inhibition was observed by an alternative FAP-binding molecule (Ft, molecule No. 4, used as assay control) or by using a protease inhibitor (PI) mixture.

Conclusion.

Binding of MpA (HFBBH), or its FAP-binding domain (F), to FAP did not affect the protease activity of FAP, as measured by its ability to cleave the fluorogenic substrate Z-GLY-PRO-AMC.

### Example 11: Comparison of Alternative Designs of Multispecific Proteins

This example describes a comparative analysis of alternative designs of multispecific proteins, in particular, the order of the FAP-binding domain, 4-1BB binding domains and HSA binding domains.

A HT1080 reporter assay was carried out in a similar fashion as described in Example 1, by assessing NF-κB activation in HT1080 cells expressing human 4-1BB co-cultured in the presence of FAP-expressing CHO cells.

Assay Setup.

NF-κB-luciferase human-4-1BB HT1080 cells as well as CHO-hFAP cells were harvested and resuspended in MEMα medium+Glutamax supplied with 10% (v/v) FBS, 1% PenStrep, 1 mg/mL G418, 100 μg/mL Normocin™ 100 μg/mL Zeocin™. Using 96-well plates, 40,000 h4-1BB-HT1080-luciferase cells and 40,000 CHO-hFAP cells were plated and increasing concentrations of multispecific protein molecules were added to the cells and incubated at 37° C., 5% CO₂. After 20 hours, supernatants were collected and centrifuged in a fresh 96-well plate. QUANTI-Luc reagent (Invivogen, Cat. No. rep-qlc1) was mixed with the supernatant and luminescence read on a Tecan M1000 luminescence plate reader. E050 values were determined by fitting the data with the four-parameter logistical fit model using Graphpad Prism software (version 7.02).

Generation of CHO Cells Expressing Human FAP.

Chinese hamster ovary (CHO) cells (ATCC® CCL121™) were transduced with plasmid pMPMPA13 containing the sequence of human fibroblast activation protein (FAP) (Uniprot accession Q12884 or NCBI Refseq. NM_004460.4). A more detailed description can be found in Example 1.

Generation of HT1080 Cells Expressing Human 4-1BB and NF-κB-Luciferase.

The fibrosarcoma cell line HT1080 (ATCC® CCL-121™) was transduced with a plasmid containing the cDNA of human 4-1BB (Myc-DDK-tagged) obtained from OriGene Technologies (#RC200664), which contains the sequence of human 4-1BB (Uniprot accession Q07011 or NCBI Refseq. NM_001561) under control of a CMV-promoter and a neomycin resistance gene. Cells were cultured in Minimum Essential Medium (MEM) α medium+Glutamax supplemented with 10% (v/v) FBS and G418 (Geneticin®). 4-1BB transduced HT1080 cells were assessed for human 4-1BB expression by flow cytometry using the mouse anti-human 4-1BB antibody clone 4B4-1 (BD Pharmingen™, Cat. No. 550890). The transfected cells were sorted by flow cytometry using the same antibody in order to enrich the population of h4-1BB expressing HT1080 cells. The h4-1BB HT1080 cells were further transfected with the NF-κB-luciferase reporter plasmid pNiFty3-N-Lucia (Invivogen, Cat. code pnf3-lc2) containing a secreted luciferase reporter gene under control of NF-κB regulated mouse interferon beta minimal promoter and a Zeocin™ resistance gene using lipofectamine. Transfected cells were cultured in Minimum Essential Medium (MEM) α medium+Glutamax supplemented with 10% (v/v) FBS, G418 (Geneticin®), Zeocin™ (Invivogen, Cat. No. ant-zn-1) & Normocin™ (Invivogen, Cat. No. ant-nr-1). A population of h4-1BB-HT1080-Lucia cells was used for the assays.

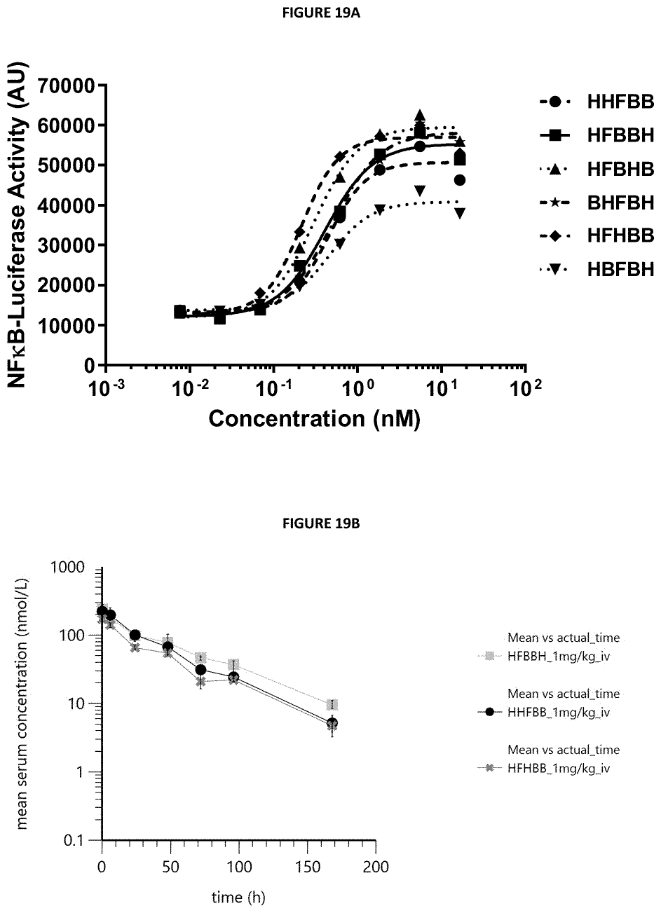

FIG. 19A shows that in the presence of FAP-expressing cells, all tested alternative designs of multispecific proteins induced 4-1BB signaling to a comparable extent, mediated by clustering via the localizer. Table 20 provides the E050 values of these alternative multispecific protein designs. FIG. 19B summarizes the results of pharmacokinetics studies in mice. Table 21 summarizes the data depicted in FIG. 19B.

The data show that, of the several alternative designs, format HFBBH (MpA) displays an improved serum half-life, while having comparable functional activity, compared to all tested format variants.

TABLE 20

| Multispecific | |
|---|---|
| protein constructs | EC50 [nM] |
| HHFBB | 0.45 |
| HFBBH | 0.41 |
| HFBHB | 0.30 |
| BHFBH | 0.51 |
| HFHBB | 0.22 |
| HBFBH | 0.46 |

TABLE 21

| parameter | unit | HFBBH | HHFBB | HFHBB |
|---|---|---|---|---|
| AUCINF_pred | h*(nmol/L) | 10673 | 9132 | 6871 |
| AUCINF_D_pred | (h*nmol*kg)/(L*mg) | 10673 | 9132 | 6871 |
| AUClast | h*(nmol/L) | 10064 | 8883 | 6644 |
| Cmax | nmol/L | 238 | 223 | 172 |
| Cmax_D | (nmol*kg)/(L*mg) | 238 | 223 | 172 |
| Tmax | h | 0.083 | 0.083 | 0.083 |
| Cl_pred | L/(h*kg) | 0.001 | 0.0014 | 0.0018 |
| Vss_pred | L/kg | 0.067 | 0.06 | 0.087 |
| HL_Lambda_z | h | 42.0 | 33.6 | 34.9 |
| AUC_% Extrap_pred | (%) | 6 | 3 | 3 |
| AUC_% Back_Ext_pred | (%) | 0 | 0 | 0 |

### Example 12: PK/PD Model Extrapolation for Human Dose Selection

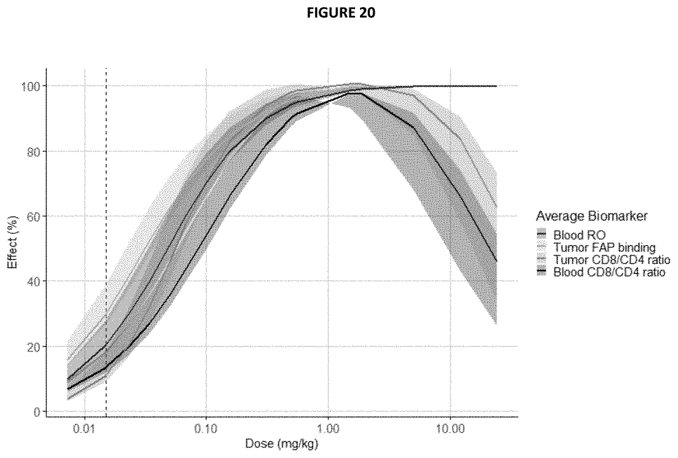

In this Example, pharmacokinetics studies predict that human half-life for MpA is from 5.9 to 14 days across a broad dose range. Predictions from a combined PK/PD model provided: (i) a human starting dose with minimal expected systemic PD effects (based on 20% receptor occupancy at 0.015 mg/kg), (ii) the anticipated therapeutic optimal dose range (0.5 to 5 mg/kg), and (iii) a dose level at which the max. therapeutic effect may be exceeded (12 mg/kg) for optimal dose range confirmation. See FIG. 20, which shows the prediction of % effect of various biomarker versus dose in human with prediction interval in shaded area.

Translational mathematical models were developed that enable the prediction of clinical pharmacokinetics, pharmacodynamics and anti-tumor effects of MpA in human based on non-clinical species data and that provide supportive justification of the Phase 1 starting dose and regimen.

The main objective of the analyses was to predict MpA pharmacokinetics (PK) and pharmacodynamics (PD) in serum/blood and tumor in humans using non-clinical species data. Non-clinical PK information in cynomolgus monkey and mouse were used to establish a minimal PBPK (mPBPK) model, assuming lumping some of the physiological compartments together, according to their endothelial vasculature, that could be translated to humans. The relationship between PK and diverse PD biomarker endpoints in mouse such as 4-1BB receptor occupancy in blood, as well as time courses of soluble 4-1BB concentrations in serum (sCD137) and changes in CD8/CD4 T cell ratios in blood/tumor were modelled, and, following incorporation of the predictions of human PK, were used to predict expected PD effects in humans.

The analysis aimed at integrating non-clinical PK and PD data to guide dose to man predictions in two parts: (i) prediction of human PK exposures in serum and tumor from mouse and monkey data through allometric scaling and mPBPK modelling; and (ii) prediction of human PD effects, using mouse-derived PKPD relationships and taking into account any human binding in vitro parameters.

Using physiologically-based parameters for the tissue volumes and lymph flows, the pharmacokinetics of MpA in mouse, monkey and humans could be described using the same approach. Translation of pharmacokinetics to humans was based on an allometrically scaled clearance, based on body-weight, and constituted the basis for prediction of the pharmacodynamics effects of MpA in humans. Furthermore, integration of data on both FAP-binding and non-FAP binding molecules into the model also enabled the characterization of the dynamics of the binding of MpA to FAP as part of this pharmacokinetic model.

The pharmacodynamic markers each were described by different pharmacokinetic-pharmacodynamic models: an Emax model for 4-1BB receptor occupancy, and a combined stimulatory and inhibitory Emax model to describe the bell-shaped dose response for the CD8/CD4 T cell ratios in blood and tumor. Each of these models could adequately describe the observed pharmacodynamics in the mouse models. Subsequently, under the assumption of similarity in pharmacodynamics in mouse and human and based on the allometric predictions of MpA concentration-time profiles in humans, predictions of human dose responses were made for each of the markers. The predictions indicated an optimal response in terms of CD8/CD4 ratio to be achieved with a dose regimen of 2 mg/kg MpA Q3W. At the proposed starting dose of 0.015 mg/kg minimal biological effects (increases in CD8/CD4 ratios of ˜20% or less) are expected. Even though some nonlinearities are anticipated in the pharmacokinetics of MpA due to saturation of target binding (predominantly FAP), these nonlinearities are predicted to occur only at higher doses (>5 mg/kg), where pharmacodynamic effects are expected to become suboptimal. Therefore, an escalation scheme with standard 3-fold increases in MpA doses between cohorts is proposed for clinical studies.

In conclusion, the analyses have provided insights of the population human mPBPK and PKPD of MpA. Results from the PD simulations suggest optimal biomarker response to be achieved at a dose of 2 mg/kg. Population PK simulations were then successfully employed to help suggest dosing every 3 weeks would be sufficient to deliver adequate average exposures to elicit maximum biomarker responses, and, given the absence of exposure boundaries around safety concerns, no additional human dosing schedules were simulated. It was concluded that MpA dose regimens of 0.015 mg/kg would be an appropriate starting dose for evaluation in a first-in-human (FIH) study in patients with solid tumors.

The specification is most thoroughly understood in light of the teachings of the references cited within the specification. The embodiments within the specification provide an illustration of embodiments of the invention and should not be construed to limit the scope of the invention. The skilled artisan readily recognizes that many other embodiments are encompassed by the invention. All publications, patents, and GenBank sequences cited in this disclosure are incorporated by reference in their entirety. To the extent the material incorporated by reference contradicts or is inconsistent with this specification, the specification will supersede any such material. The citation of any references herein is not an admission that such references are prior art to the present invention.

Those skilled in the art will recognize, or be able to ascertain using no more than routine experimentation, many equivalents to the specific embodiments of the invention described herein. Such equivalents are intended to be encompassed by the following embodiments.

1. A recombinant protein comprising:
a first ankyrin repeat domain that specifically binds fibroblast activation protein (FAP), a second ankyrin repeat domain that specifically binds 4-1BB, a third ankyrin repeat domain that specifically binds 4-1BB, a fourth ankyrin repeat domain that specifically binds serum albumin, and a fifth ankyrin repeat domain that specifically binds serum albumin,
wherein said ankyrin repeat domains are arranged, from the N-terminus to C-terminus, according to the following formula: (serum albumin binding domain)-(FAP-binding domain)-(4-1BB binding domain)-(4-1BB binding domain)-(serum albumin binding domain).

2. The recombinant protein of claim 1, wherein said FAP binding domain comprises an amino acid sequence that is at least 90% identical to SEQ ID NO:2, and binds human FAP with a K_{D} value of or below 10 nM.

3. The recombinant protein of claim 1, wherein said FAP binding domain comprises the amino acid sequence of SEQ ID NO: 2.

4. The recombinant protein of claim 1, wherein each of said 4-1BB binding domains independently comprises an amino acid sequence that is at least 90% identical to SEQ ID NO:3, and binds human 4-1BB with a K_{D} value of or below 10 nM.

5. The recombinant protein of claim 1, wherein each of said 4-1BB binding domains comprises the amino acid sequence of SEQ ID NO: 3.

6. The recombinant protein of claim 1, wherein said N-terminal serum albumin binding domain comprises an amino acid sequence that is at least 90% identical to SEQ ID NO:5, and binds human serum albumin with a K_{D} value of or below 10 nM.

7. The recombinant protein of claim 1, wherein the N-terminal serum albumin domain comprises the amino acid sequence of SEQ ID NO: 5.

8. The recombinant protein of claim 1, wherein said C-terminal serum albumin binding domain comprises an amino acid sequence that is at least 90% identical to SEQ ID NO:1, and binds human serum albumin with a K_{D} value of or below 10 nM.

9. The recombinant protein of claim 1, wherein the C-terminal serum albumin domain comprises the amino acid sequence of SEQ ID NO: 1.

10. The recombinant protein of claim 1, comprising the following formula, from the N-terminus to C-terminus: (serum albumin binding domain)-(linker)-(FAP-binding domain)-(linker)-(4-1BB binding domain)-(linker)-(4-1BB binding domain)-(linker)-(serum albumin binding domain), wherein the linker comprises the amino acid sequence of SEQ ID NO: 4.

11. A recombinant protein comprising the amino acid sequence of SEQ ID NO: 6.

12. A recombinant protein comprising an amino acid sequence that is at least 90% identical to SEQ ID NO: 6, and binds human FAP, human 4-1BB, and human serum albumin with a K_{D} value of or below 10 nM.

13. The recombinant protein of claim 1, wherein said protein has a half maximal effective concentration (E050) of from about 0.1 nM to about 5 nM, as assessed by an in vitro IFNγ release assay.

14. A pharmaceutical composition comprising a recombinant protein of claim 1, and a pharmaceutically acceptable carrier or excipient.

15. An isolated nucleic acid molecule encoding the recombinant protein of claim 1.

16. A host cell comprising the nucleic acid molecule of claim 15.

17. A method of making the recombinant protein of claim 1, comprising culturing the host cell of claim 16 under conditions wherein said recombinant protein is expressed.

18. A method of treating cancer, the method comprising administering to a subject in need thereof an effective amount of the recombinant protein of claim 1.

19. The method of claim 18, wherein said subject is a human.

20. The method of claim 18, wherein said cancer is a solid tumor.
